SEQUENCE LISTING (111 sequences)

SEQ ID NO: 1 - protein, 45 aa, Homo Sapien
TINVHDVETQFNQYKTEAASRYNLTISDVSVSDVPFPFSAQSGAG

SEQ ID NO: 2 - protein, 426 aa, Homo Sapien
MRQVCCSALPPPPLEKGRCSSYSDSSSSSSERSSSSSSSSSESGSSSRSSSNNSSISRPAAPPEPRPQQQPQPRSPAARRAAARSRAAAAGGMRRDPAPGFSMLLFGVSLACYSPSLKSVQDQAYKAPVVVEGKVQGLVPAGGSSSNSTREPPASGRVALVKVLDKWPLRSGGLQREQVISVGSCVPLERNQRYIFFLEPTEQPLVFKTAFAPLDTNGKNLKKEVGKILCTDCATRPKLKKMKSQTGQVGEKQSLKCEAAAGNPQPSYRWFKDGKELNRSRDIRIKYGNGRKNSRLQFNKVKVEDAGEYVCEAENILGKDTVRGRLYVNSVSTTLSSWSGHARKCNETAKSYCVNGGVCYYIEGINQLSCKCPNGFFGQRCLEKLPLRLYMPDPKQSVLWDTPGTGVSSSQWSTSPKPRSCTRRGS

SEQ ID NO: 3 - protein, 422 aa, Homo Sapien
MRQVCCSALPPPPLEKGRCSSYSDSSSSSSERSSSSSSSSSESGSSSRSSSNNSSISRPAAPPEPRPQQQPQPRSPAARRAAARSRAAAAGGMRRDPAPGFSMLLFGVSLACYSPSLKSVQDQAYKAPVVVEGKVQGLVPAGGSSSNSTREPPASGRVALVKVLDKWPLRSGGLQREQVISVGSCVPLERNQRYIFFLEPTEQPLVFKTAFAPLDTNGKNLKKEVGKILCTDCATRPKLKKMKSQTGQVGEKQSLKCEAAAGNPQPSYRWFKDGKELNRSRDIRIKYGNGRKNSRLQFNKVKVEDAGEYVCEAENILGKDTVRGRLYVNSVSTTLSSWSGHARKCNETAKSYCVNGGVCYYIEGINQLSCKCPNGFFGQRCLEKLPLRLYMPDPKQSVLWDTPGTGVSSSQWSTSPSTLDLN

SEQ ID NO: 4 - protein, 17 aa, ARTIFICIAL
  SYNTHESIZED SEQUENCE
HDVETQFNQYKTEAASC

SEQ ID NO: 5 - protein, 16 aa, ARTIFICIAL
  SYNTHESIZED SEQUENCE
HDVETQFNQYKTEAAS

SEQ ID NO: 6 - protein, 17 aa, ARTIFICIAL
  SYNTHESIZED SEQUENCE
QYKTEAASRYNLTISDC

SEQ ID NO: 7 - protein, 19 aa, ARTIFICIAL
  SYNTHESIZED SEQUENCE
SDVSVSDVPFPFSAQSGAC

SEQ ID NO: 8 - protein, 19 aa, ARTIFICIAL
  SYNTHESIZED SEQUENCE
DVETQFNQYKTEAASRYNL

SEQ ID NO: 9 - protein, 20 aa, ARTIFICIAL
  SYNTHESIZED SEQUENCE
NIKFRPGSVVVQLTLAFREC

SEQ ID NO: 10 - DNA, 1054 bp, Homo Sapien
ccctgcagaagccgcgctccctactcggtccgggggcagagggggcgcgagagagcaagtgggcgggcgtcccatcctccgcatcctcctccaggtcctggcgcacagggtgggagcgctgcgctgcgccgcgctgcgcatcgcggcccgcttgccgcctgccccctgccctagctgggccacctccccgggctgccggtggagggctaagaggcgctaacgttacgctgtttccggttttccagcgggctctgtttcccctcccaaggcggcggcggctgagcggcggagccccccaaatggcctggccagatgcggcaggtttgctgctcagcgctgccgccgccgccactggagaagggtcggtgcagcagctacagcgacagcagcagcagcagcagcgagaggagcagcagcagcagcagcagcagcagcgagagcggcagcagcagcaggagcagcagcaacaacagcagcatctctcgtcccgctgcgcccccagagccgcggccgcagcaacagccgcagccccgcagccccgcagcccggagagccgccgcccgttcgcgagccgcagccgccggcggcatgaggcgcgacccggcccccggcttctccatgctgctcttcggtgtgtcgctcgcctgctactcgcccagcctcaagtcagtgcaggaccaggcgtacaaggcacccgtggtggtggagggcaaggtacaggggctggtcccagccggcggctccagctccaacagcacccgagagccgcccgcctcgggtcgggtggcgttggtaaaggtgctggacaagtggccgctccggagcggggggctgcagcgcgagcaggtgatcagcgtgggctcctgtgtgccgctcgaaaggaaccagcgctacatctttttcctggagcccacggaacagcccttagtctttaagacggcctttgcccccctcgataccaacggcaaaaatctcaagaaagaggtgggcaagatcctgtgcactgactgcggtgagtcgccccctccctttgctggagaaaggggggaggggc

SEQ ID NO: 11 - DNA, 419 bp, Homo Sapien
gcactgcccctaccaccaccgtgctcacctacctgccttgtaacccacagccacccggcccaagttgaagaagatgaagagccagacgggacaggtgggtgagaagcaatcgctgaagtgtgaggcagcagccggtaatccccagccttcctaccgttggttcaaggatggcaaggagctcaaccgcagccgagacattcgcatcaaatatggcaacggcaggtaagcaatatccagcctgtcctccatccaagcagctgccctccctcctctcgagaaggagtggcctgaactgggcagaggccagggctgtgcccccagactcacccatggaaccagtcccagcccctctccatactccccttccttgccactcccagccttcttcctgcagctcggggaccctctggtcatggtgg

SEQ ID NO: 12 - DNA, 493 bp, Homo Sapien
gccactagtcctgccctgtgttgtcctaagggcccaaggctccctgcagctgcagggactccgtgccccacctggactccagaaattgctggggggagggaatctccttcccatctaagctgcagggccactgtggctctgtggagagaggcaaccgctgggtgactgctggggagccacagccggccctggctcacgcctctcccccttttatcccctccctaaccagaaagaactcacgactacagttcaacaaggtgaaggtggaggacgctggggagtatgtctgcgaggccgagaacatcctggggaaggacaccgtccggggccggctttacgtcaacagcggtaggtgggcccagacagagggaagggtcctagaggggtttggcagggcgggccagtggccccccagcccttggggcctgcatgtgatccatctcgcactcaactcccctccctcctgctgctgccatatcaagccctagatctc

SEQ ID NO: 13 - DNA, 350 bp, Homo Sapien
ggtcatcttccagttttgacgtggggcatgaaggagatgattcctggggcctagggatagtctcagtgcgtcactggcacatgtctctcataccctcagtgagcaccaccctgtcatcctggtcggggcacgcccggaagtgcaacgagacagccaagtcctattgcgtcaatggaggcgtctgctactacatcgagggcatcaaccagctctcctgcaagtaagtgaccagtaggggtgggcatgggagcaagaacagggtaggagatgctgggtcagaagtggagggctctaggaaaagagggttccaagccactgacaagaggtccccaaggggtgtagacaggaag

SEQ ID NO: 14 - DNA, 326 bp, Homo Sapien
aagtgcctgacttggtttttctcattcatcactgtgcactttgatttctaaccttagcctcgcggatgatgccagtttgttctgtgatttttccccttttccttctccagatgtccaaatggattcttcggacagagatgtttggagaaactgcctttgcgattgtacatgccagatcctaagcaaagtatgtttgaagatacaaacacaagtccctgctccttagaaagcttctgggtgcagtccccccaatgtagggcatagggccaggggtgttggttgttttgatctttggctgttttgttttgtttctcttttcttttttg

SEQ ID NO: 15 - DNA, 310 bp, Homo Sapien
aatacaaatggtaacggtggcaaggaacccagctggcattggcaggagcccgtgccggggtgttgcaatgcctgtgtcttgctatcctggctctctctttctggttttctttcttttggtaggtgtcctgtgggatacaccggggacaggtgtcagcagttcgcaatggtcaacttctccagtatgtctctttcttctatttcttctcttccctggtgactgacagtctccccctggggagggatggggccttgcttagcaagactagggggagatgtagagggtgtcctgcgctccttagaggagacgg

SEQ ID NO: 16 - DNA, 259 bp, Homo Sapien
ggatggaaaggagctgggcaggacctgagctggggagagcctatggctttgggggcaaaggggtctctgcaccactatccctatggggtttgcctcctgccccacccctcacatctaccctacctctgcagaagccgaggagctgtaccagaagagggtcctgaccatcacgggcatctgcgtggctctgctggtcgtgggcatcgtctgtgtggtggcctactgcaagaccaagtgagtgtcactcactcaagctggg

SEQ ID NO: 17 - DNA, 258 bp, Homo Sapien
ttgtgaccctcgctcttctgcactgccagagcaccttggatttgaattaaagggtatgaaacctctgtgtgtcactcagcaccatctgcgtggcccctctccacagcctccgatgccatgtgtgctgaatgtttggtttcctttccaaaaaatggaaaagggatgattccaaaaatctctcctggccccatcgagatgtcctatcataaactacgttgatttagcaggagacgaaagaagctcatgaacagaagggca

SEQ ID NO: 18 - DNA, 8181 bp, Homo Sapien
  misc_feature (6890)..(7960): definition of “n” in this sequence is unavailable at this time
gaattcagaattttagaccctttggccttggggtccatcctggagaccctgaggtctaagctacagcccctcagccaaccacagacccttctctggctcccaaaaggagttcagtcccagagggtggtcacccacccttcagggatgagaagttttcaaggggtattactcaggcactaaccccaggaaagatgacagcacattgccataaagttttggttgttttctaagccagtgcaactgcttattttagggattttccgggatagggtggggaagtggaaggaatcggcgagtagaagagaaagcctgggagggtggaagttagggatctaggggaagtttggctgatttggggatgcgggtgggggaggtgctggatggagttaagtgaaggatagggtgcctgagggaggatgcccgaagtcctcccagacccacttactcacggtggcagcggcgacactccagtctatcaaagatccgccgggatggagagccaggaggcgggggctgcccctgaggtagcggggaggccggggggccggggggcggacgggacgagtgcaatattggcgggggaaaaaacaacactgcaccgcgtcccgtccctcccgcccgcccgggcccggatcccgctccccaccgcctgaagccggcccgacccggaacccgggccgctggggagttgggttcaccttggaggccagagagacttggcgcccggaagcaaagggaatggcaagggggaggggggagggagaacgggagtttgcggagtccagaaggccgctttccgacgcccgggcgttgcgcgcgcttgctctttaagtactcagactgcgcggcgcgagccgtccgcatggtgacgcgtgtcccagcaaccgaactgaatggctgttgcttggcaatgccgggagttgaggtttggggccgcccacctagctactcgtgttttctccggcctgcgagttggggggctcccgcctccccggcccgctcctgggcgcgctgacgtcagatgtccccaccccgcccagcgcctgccccaagggtctcgccgcacacaaagctcggcctcgggcgccggcgcgcgggcgagagcggtggtctctcgcctgctgatctgatgcgctccaatcccgtgcctcgccgaagtgtttttaaagtgttctttccaacctgtgtctttggggctgagaactgttttctgaatacaggcggaactgcttccgtcggcctagaggcacgctgcgactgcgggacccaagttccacgtgctgccgcggcctgggatagcttcctcccctcgtgcactgctgccgcacacacctcttggctgtcgcgcattacgcacctcacgtgtgcttttgccccccgctacgtgcctacctgtccccaataccactctgctccccaaaggatagttctgtgtccgtaaatcccattctgtcaccccacctactctctgccccccccttttttgttttgagacggagctttgctctgtcgcccaggctggagtgcaatggcgcgatctcggctcactgcaacctccgcctcccgggttcaagcgattctcctgcctcagcctcctgagtagctggggttacagcgcccgccaccacgctcggctaatttttgtagtttttagtagagacgaggtttcaccatcttggccaggctggtcttgaacccctgaccttgtgatccactcgcctcggccttccaaagtgttgggattacgggcgtgacgaccgtgccacgcatctgcctcttaagtacataacggcccacacagaacgtgtccaactcccccgcccacgttccaacgtcctctcccacatacctcggtgccccttccacatacctcaggaccccacccgcttagctccatttcctccagacgccaccaccacgcgtcccggagtgccccctcctaaagctcccagccgtccaccatgctgtgcgttcctccctccctggccacggcagtgacccttctctcccgggccctgcttccctctcgcgggctctgctgcctcacttaggcagcgctgcccttactcctctccgcccggtccgagcggcccctcagcttcggcgcccagccccgcaaggctcccggtgaccactagagggcgggaggagctcctggccagtggtggagagtggcaaggaaggaccctagggttcatcggagcccaggtttactcccttaagtggaaatttcttcccccactcctccttggctttctccaaggagggaacccaggctgctggaaagtccggctgggggggggactgtgggttcaggggagaacggggtgtggaacgggacagggagcggttagaagggtggggctattccgggaagtggtggggggagggagcccaaaactagcacctagtccactcattatccagccctcttatttctcggccgctctgcttcagtggacccggggagggcggggaagtggagtgggagacctaggggtgggcttcccgaccttgctgtacaggacctcgacctagctggctttgttccccatccccacgttagttgttgccctgaggctaaaactagagcccaggggccccaagttccagactgcccctcccccctcccccggagccagggagtggttggtgaaagggggaggccagctggagaacaaacgggtagtcagggggttgagcgattagagcccttgtaccctacccaggaatggttggggaggaggaggaagaggtaggaggtaggggagggggcggggttttgtcacctgtcacctgctcgctgtgcctagggcgggcgggcggggagtggggggaccggtataaagcggtaggcgcctgtgcccgctccacctctcaagcagccagcgcctgcctgaatctgttctgccccctccccacccatttcaccaccaccatgacaccgggcacccagtctcctttcttcctgctgctgctcctcacagtgcttacaggtgaggggcacgaggtggggagtgggctgccctgcttaggtggtcttcgtggtctttctgtgggttttgctccctggcagatggcaccatgaagttaaggtaagaattgcagacagaggctgccctgtctgtgccagaaggagggagaggctaaggacaggctgagaagagttgcccccaaccctgagagtgggtaccaggggcaagcaaatgtcctgtagagaagtctagggggaagagagtagggagagggaaggcttaagaggggaagaaatgcaggggccatgagccaaggcctatgggcagagagaaggaggctgctgcagggaaggaggcttccaacccaggggttactgaggctgcccactccccagtcctcctggtattatttctctggtggccagagcttatattttcttcttgctcttatttttccttcataaagacccaaccctatgactttaacttcttacagctaccacagcccctaaacccgcaacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatggtgagtatcggcctttccttccccatgctcccctgaagcagccatcagaactgtccacaccctttgcatcaagcccgagtcctttccctctcaccccagtttttgcagatttataaacaagggggttttctgggcctctccaatattaagttcaggtacagttctgggtgtggacccagtgtggtggttggagggttgggtggtggtcatgaccgtaggagggactggtgcacttaaggttgggggaagagtgctgagccagagctgggacccgtggctgaagtgcccatttccctgtgaccaggccaggatctgtggtggtacaattgactctggccttccgagaaggtaccatcaatgtccacgacgtggagacacagttcaatcagtataaaacggaagcagcctctcgatataacctgacgatctcagacgtcagcggtgaggctacttccctggctgcagccagcaccatgccggggcccctctccttccagtgtctgggtccccgctctttccttagtgctggcagcgggaggggcgcctcctctgggagactgccctgaccactgcttttccttttagtgagtgatgtgccatttcctttctctgcccagtctggggctggggtgccaggctggggcatcgcgctgctggtgctggtctgtgttctggttgcgctggccattgtctatctcattgccttggtgagtgcagtccctggccctgatcagagccccccggtagaaggcactccatggcctgccataacctcctatctccccaggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtgagattgggccccacaggccaggggaagcagagggtttggctgggcaaggattctgaagggggtacttggaaaacccaaagagcttggaagaggtgagaagtggcgtgaagtgagcaggggagggcctggcaaggatgaggggcagaggtcagaggagttttgggggacaggcctgggaggagactatggaagaaaggggccctcaagagggagtggccccactgccagaattcctaaaagatcattggccgtccacattcatgctggctggcgctggctgaactggtgccaccgtggcagttttgttttgttttgcttttttgcacccagaggcaaaatgggtggagcactatgcccaggggagcccttcccgaggagtcccaggggtgagcctctgtgcccctaatcatctcctaggaatggagggtagaccgagaaaggctggcatagggggaggtttcccaggtagaagcaagaagaagtgtcagcagaccaggtgagcgtgggtgccagtggggttcttgggagcttcaaggaagcaaggaacgctccctccttcctctcctggtctttctctatgggacctagtaaataattactgcagccacctgaggctggaaaaccactccaggtgggggaggagagagtttagttttcttgctcctattttcctcctcctggagacctccctctctcggctttacaaagacacagatacaccccgccccccaaaacacacacacacacacacacacacacacctccttaggctggaacagcagagaatggagggacaagggggctgattagagccaagaagagggagtgaaggagagcagagggaggagggcagccctgtttacagtcacctggctggtggggtggcaggtgctctctctgaattaaccctttgagagctggccaggactctggactgattaccccagcctggggtggcatccaggggctctaggaggtaccttttgctcctcaccctggatctcttttccttccacccaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacacgtccttcagaggccccaccaatttctcggacacttctcagtgtgtggaagctcatgtgggcccctgaggctcatgcctgggaagtgttgtggtgggggctcccaggaggactggcccagagagccctgagatagcggggatcctgaactggactgaataaaacgtggtctcccactggcgccaacttctgatctttcatctgtgacccgtgggcagcagggcgtcagaatgtgtgtgagggggctgggggaggagacagggaggccaggaggcagtaaggagcgagtttgtttgagaagcaggagatgtgaggaggaggtgacattggggagtaggggtggcctgaggagccacctctggctaaccctggcagcacaagaggaaggaggaaacgaaacccaggcnggctttggagggctagcgtgactgggctccgtgactgagctctgtgtgccagtggctctcccctctcctcgcctggcccacgccctccttgcccctggcatggtgccccccaggtggctctattcttagctgtccgggtgtgaagtaaatccttgggcagtgataacagcccagagtcaacagggttgagataagcagaggctgggtcagatccgggcgctggcaccaggcccagccccctccctgaccccggctnccccaccagcctgctgcccctggggtggnctccacaacaccctgggaatggggaagtggttctggttccctgacccctttggcccaggcacgttgcctgtccctcgaccgcattcccccagggcctgtgctgcaggcctggaagccctgattggggcctgccaccagcagccagagagctatgttccctggcagctgtgatgcgctcaggccgggccaggacacgtgtggcaggaggcttagagcacctgcctggggccttcctctctcaggcaccagatccattggttgctcctgcctagaaccacagcctagcacccctgctccctcccgcctaccacacccagcacagaaactcacaggaatgattgcgctcagggaaggcagagatgtgcctggcatcacagtttattgtttataaaccatgacaataacagctgttgctcagcacaggcctagcagagcccactgcagggggacggcagcgggcaccagaggccttgcctggcccaacccaatgggaacacccagactcagctgggtccccaagggagacttggcacattggcatgggtgtgggacaggtaaagcatgcaagagggagaagagggacataaggggcatgcggctgcggggtgttgggacccaaataaataaagcaggatgacagggtccccttcccctcaccaggaatgcctgacagcgtccagccccaaagcctgcctgtcccaaggctgtagttcagcatcaacagggcagggagcttggcagggcaagggcagagctggagatcatgcccagtnttccaggtgccctccctcccaatcagcctggggggcacaggacagggatggagaaggggctctctccatggcttgggtaacatgccaaaggcaggtcatagggcagactcagtgggggtgggggcctggctaacaagcaatggagagaacgggggccatccagagaggttggcagaagagagcccctgggtcaagagaaaactttggggaagacaagacacgggagaag

SEQ ID NO: 19 - RNA, 1799 nt, RNA
ccaccucucaagcagccagcgccugccugaaucuguucugcccccuccccacccauuucaccaccaccaugacaccgggcacccagucuccuuucuuccugcugcugcuccucacagugcuuacaguuguuacagguucuggucaugcaagcucuaccccagguggagaaaaggagacuucggcuacccagagaaguucagugcccagcucuacugagaagaaugcugugaguaugaccagcagcguacucuccagccacagccccgguucaggcuccuccaccacucagggacaggaugucacucuggccccggccacggaaccagcuucagguucagcugccaccuggggacaggaugucaccucggucccagucaccaggccagcccugggcuccaccaccccgccagcccacgaugucaccucagccccggacaacaagccagccccgggcuccaccgcccccccagcccacggugucaccucggccccggacaccaggccggccccgggcuccaccgcccccccagcccauggugucaccucggccccggacaacaggcccgccuugggcuccaccgccccuccaguccacaaugucaccucggccucaggcucugcaucaggcucagcuucuacucuggugcacaacggcaccucugccagggcuaccacaaccccagccagcaagagcacuccauucucaauucccagccaccacucugauacuccuaccacccuugccagccauagcaccaagacugaugccaguagcacucaccauagcacgguaccuccucucaccuccuccaaucacagcacuucuccccaguugucuacuggggucucuuucuuuuuccugucuuuucacauuucaaaccuccaguuuaauuccucucuggaagaucccagcaccgacuacuaccaagagcugcagagagacauuucugaaauguuuuugcagauuuauaaacaaggggguuuucugggccucuccaauauuaaguucaggccaggaucuguggugguacaauugacucuggccuuccgagaagguaccaucaauguccacgacguggagacacaguucaaucaguauaaaacggaagcagccucucgauauaaccugacgaucucagacgucagcgugagugaugugccauuuccuuucucugcccagucuggggcuggggugccaggcuggggcaucgcgcugcuggugcuggucuguguucugguugcgcuggccauugucuaucucauugccuuggcugucugucagugccgccgaaagaacuacgggcagcuggacaucuuuccagcccgggauaccuaccauccuaugagcgaguaccccaccuaccacacccaugggcgcuaugugcccccuagcaguaccgaucguagccccuaugagaagguuucugcagguaaugguggcagcagccucucuuacacaaacccagcaguggcagccacuucugccaacuuguaggggcacgucgcccgcugagcugaguggccagccagugccauuccacuccacucagguucuucagggccagagccccugcacccuguuugggcuggugagcugggaguucaggugggcugcucacacguccuucagaggccccaccaauuucucggacacuucucaguguguggaagcucaugugggccccugaggcucaugccugggaaguguuguggugggggcucccaggaggacuggcccagagagcccugagauagcggggauccugaacuggacugaauaaaacguggucucccacug

SEQ ID NO: 20 - RNA, 1428 nt, RNA
augacaccgggcacccagucuccuuucuuccugcugcugcuccucacagugcuuacaguuguuacagguucuggucaugcaagcucuaccccagguggagaaaaggagacuucggcuacccagagaaguucagugcccagcucuacugagaagaaugcugugaguaugaccagcagcguacucuccagccacagccccgguucaggcuccuccaccacucagggacaggaugucacucuggccccggccacggaaccagcuucagguucagcugccaccuggggacaggaugucaccucggucccagucaccaggccagcccugggcuccaccaccccgccagcccacgaugucaccucagccccggacaacaagccagccccgggcuccaccgcccccccagcccacggugucaccucggccccggacaccaggccggccccgggcuccaccgcccccccagcccauggugucaccucggccccggacaacaggcccgccuugggcuccaccgccccuccaguccacaaugucaccucggccucaggcucugcaucaggcucagcuucuacucuggugcacaacggcaccucugccagggcuaccacaaccccagccagcaagagcacuccauucucaauucccagccaccacucugauacuccuaccacccuugccagccauagcaccaagacugaugccaguagcacucaccauagcacgguaccuccucucaccuccuccaaucacagcacuucuccccaguugucuacuggggucucuuucuuuuuccugucuuuucacauuucaaaccuccaguuuaauuccucucuggaagaucccagcaccgacuacuaccaagagcugcagagagacauuucugaaauguuuuugcagauuuauaaacaaggggguuuucugggccucuccaauauuaaguucaggccaggaucuguggugguacaauugacucuggccuuccgagaagguaccaucaauguccacgacguggagacacaguucaaucaguauaaaacggaagcagccucucgauauaaccugacgaucucagacgucagcgugagugaugugccauuuccuuucucugcccagucuggggcuggggugccaggcuggggcaucgcgcugcuggugcuggucuguguucugguugcgcuggccauugucuaucucauugccuuggcugucugucagugccgccgaaagaacuacgggcagcuggacaucuuuccagcccgggauaccuaccauccuaugagcgaguaccccaccuaccacacccaugggcgcuaugugcccccuagcaguaccgaucguagccccuaugagaagguuucugcagguaaugguggcagcagccucucuuacacaaacccagcaguggcagccacuucugccaacuuguag

SEQ ID NO: 21 - DNA, 21 bp, ARTIFICIAL
  Target MUC1 RNA Sequence for siRNA
aaaaggagacttcggctaccc

SEQ ID NO: 22 - DNA, 21 bp, ARTIFICIAL
  Synthesized Sequence
aaggagacuucggcugccctt

SEQ ID NO: 23 - DNA, 21 bp, ARTIFICIAL
  SYNTHESIZED SEQUENCE
gggcagccgaagucuccuutt

SEQ ID NO: 24 - DNA, 21 bp, ARTIFICIAL
  TARGET MUC1 SEQUENCE FOR siRNA
aaaggagacttcggctaccca

SEQ ID NO: 25 - DNA, 21 bp, ARTIFICIAL
  SYNTHESIZED SEQUENCE
aggagacuucggcuacccatt

SEQ ID NO: 26 - DNA, 21 bp, ARTIFICIAL
  SYNTHESIZED SEQUENCE
uggguagccgaagucuccutt

SEQ ID NO: 27 - DNA, 21 bp, ARTIFICIAL
  TARGET MUC1 RNA SEQUENCE FOR siRNA
aaggagacttcggctacccag

SEQ ID NO: 28 - DNA, 21 bp, ARTIFICIAL
  SYNTHESIZED SEQUENCE
ggagacuucggcuacccagtt

SEQ ID NO: 29 - DNA, 21 bp, ARTIFICIAL
  SYNTHETIC SEQUENCE
cuggguagccgaagucucctt

SEQ ID NO: 30 - DNA, 21 bp, ARTIFICIAL
  TARGET MUC1 RNA SEQUENCE FOR siRNA
aaccagcttcaggttcagctg

SEQ ID NO: 31 - DNA, 21 bp, ARTIFICIAL
  SYNTHESIZED SEQUENCE
ccagcuucagguucagcugtt

SEQ ID NO: 32 - DNA, 21 bp, ARTIFICIAL
  SYNTHESIZED SEQUENCE
cagcugaaccugaagcuggtt

SEQ ID NO: 33 - DNA, 21 bp, ARTIFICIAL
  TARGET MUC1 RNA SEQUENCE FOR siRNA
aacggcacctctgccagggct

SEQ ID NO: 34 - DNA, 21 bp, ARTIFICIAL
  SYNTHESIZED SEQUENCE
cggcaccucugccagggcutt

SEQ ID NO: 35 - DNA, 21 bp, ARTIFICIAL
  SYNTHESIZED SEQUENCE
agcccuggcagaggugccgtt

SEQ ID NO: 36 - DNA, 21 bp, Homo Sapien
aagactgatgccagtagcact

SEQ ID NO: 37 - DNA, 21 bp, ARTIFICIAL
  Synthesized Sequence
gacugaugccaguagcacutt

SEQ ID NO: 38 - DNA, 21 bp, ARTIFICIAL
  Synthesized Sequence
agugcuacuggcaucaguctt

SEQ ID NO: 39 - DNA, 21 bp, Homo Sapien
aattgactctggccttccgag

SEQ ID NO: 40 - DNA, 21 bp, ARTIFICIAL
  Synthesized Sequence
uugacucuggccuuccgagtt

SEQ ID NO: 41 - DNA, 20 bp, ARTIFICIAL
  Synthesized Sequence
cucggaaggccagagucatt

SEQ ID NO: 42 - DNA, 21 bp, Homo Sapien
aaggtaccatcaatgtccacg

SEQ ID NO: 43 - DNA, 21 bp, ARTIFICIAL
  Synthesized Sequence
gguaccaucaauguccacgtt

SEQ ID NO: 44 - DNA, 21 bp, ARTIFICIAL
  Synthesized Sequence
cguggacauugaugguacctt

SEQ ID NO: 45 - DNA, 21 bp, Homo Sapien
aatgtccacgacgtgaagaca

SEQ ID NO: 46 - DNA, 21 bp, ARTIFICIAL
  Synthesized Sequence
uguccacgacgugaagacatt

SEQ ID NO: 47 - DNA, 21 bp, ARTIFICIAL
  Synthesized Sequence
ugucuucacgucguggacatt

SEQ ID NO: 48 - DNA, 21 bp, Homo Sapien
aatcagtataaaacggaggca

SEQ ID NO: 49 - DNA, 21 bp, ARTIFICIAL
  Synthsized Sequence
ucaguauaaaacggaggcatt

SEQ ID NO: 50 - DNA, 21 bp, ARTIFICIAL
  Sythesized Sequence
ugccuccguuuuauacugatt

SEQ ID NO: 51 - DNA, 21 bp, Homo Sapien
aaaacggaagcagcctctcga

SEQ ID NO: 52 - DNA, 21 bp, ARTIFICIAL
  Synthesized Sequence
aacggaagcagccucucggtt

SEQ ID NO: 53 - DNA, 21 bp, ARTIFICIAL
  Synthesized Sequence
ucgacaggcugcuuccguutt

SEQ ID NO: 54 - DNA, 21 bp, Homo Sapien
aaacggaagcagcctctcgat

SEQ ID NO: 55 - DNA, 21 bp, ARTIFICIAL
  Synthesized Sequence
acggaagcagccucucgautt

SEQ ID NO: 56 - DNA, 21 bp, ARTIFICIAL
  Synthesized Sequence
aucgagaggcugcuuccgutt

SEQ ID NO: 57 - DNA, 21 bp, Homo Sapien
aacggaagcagcctctcgata

SEQ ID NO: 58 - DNA, 21 bp, ARTIFICIAL
  Synthesized Sequence
cggaagcagccucucgauatt

SEQ ID NO: 59 - DNA, 21 bp, ARTIFICIAL
  Synthesized Sequence
uaucgagaggcugcuuccgtt

SEQ ID NO: 60 - DNA, 21 bp, Homo Sapien
aagaactacgggcagctggac

SEQ ID NO: 61 - DNA, 21 bp, ARTIFICIAL
  Synthesized Sequence
gaccuacgggcagcuggactt

SEQ ID NO: 62 - DNA, 21 bp, ARTIFICIAL
  Synthesized Sequence
auccagcugcccguaguuctt

SEQ ID NO: 63 - DNA, 21 bp, Homo Sapien
aagggggttttctgggcctct

SEQ ID NO: 64 - DNA, 21 bp, ARTIFICIAL
  Synthesized Sequence
ggggguuuucugggccucutt

SEQ ID NO: 65 - DNA, 21 bp, ARTIFICIAL
  Synthesized Sequence
agaggcccagaaaaccccctt

SEQ ID NO: 66 - DNA, 21 bp, DNA
aagttcagtgcccagctctac

SEQ ID NO: 67 - DNA, 21 bp, Homo Sapien
guucagugcccagcucuactt

SEQ ID NO: 68 - DNA, 21 bp, Homo Sapien
guagagcugggcacugaactt

SEQ ID NO: 69 - DNA, 21 bp, Homo Sapien
aaggtttctgcaggtaacggt

SEQ ID NO: 70 - DNA, 21 bp, Homo Sapien
gguuucugcagguaauggutt

SEQ ID NO: 71 - DNA, 21 bp, Homo Sapien
accauuaccugcagaaacctt

SEQ ID NO: 72 - DNA, 19 bp, Homo Sapien
gcgcgctttgaaggattcg

SEQ ID NO: 73 - DNA, 21 bp, Homo Sapien
gcgcgcuuuguaggauucgtt

SEQ ID NO: 74 - DNA, 21 bp, Homo Sapien
cgaauccuacaaagcgcgctt

SEQ ID NO: 75 - RNA, 1424 nt, ARTIFICIAL
  Synthesized Sequence
caaguuggcagaaguggcugccacugcuggguuuguguaagagaggcugcugccaccauuaccugcagaaaccuucucauaggggcuacgaucgguacugcuagggggcacauagcgcccaugggugugguaggugggguacucgcucauaggaugguagguaucccgggcuggaaagauguccagcugcccguaguucuuucggcggcacugacagacagccaaggcaaaugagauagacaaugccagcgcaaccagaacacagaccagcaccagcagcgcgaugccccagccuggcaccccagccccagacugggcagagaaaggaaauggcacaucacucacgcugacgucugagaucgucagguuauaucgagaggcugcuuccguuuuauacugauugaacugugucuccacgucguggacauugaugguaccuucucggaaggccagagucaauuguaccaccacagauccuggccugaacuuaauauuggagaggcccagaaaacccccuuguuuauaaaucugcaaaaacauuucagaaaugucucucugcagcucuugguaguagucggugcugggaucuuccagagaggaauuaaacuggagguuugaaaugugaaaagacaggaaaaagaaagagaccccaguagacaacuggggagaagugcugugauuggaggaggugagaggagguaccgugcuauggugagugcuacuggcaucagucuuggugcuauggcuggcaagggugguaggaguaucagagugguggcugggaauugagaauggagugcucuugcuggcugggguugugguagcccuggcagaggugccguugugcaccagaguagaagcugagccugaugcagagccugaggccgaggugacauuguggacuggaggggcgguggagcccaaggcgggccuguuguccggggccgaggugacaccaugggcugggggggcgguggagcccggggccggccugguguccggggccgaggugacaccgugggcugggggggcgguggagcccggggcuggcuuguuguccggggcugaggugacaucgugggcuggcggggugguggagcccagggcuggccuggugacugggaccgaggugacauccugucccagguggcagcugaaccugaagcugguuccguggccggggccagagugacauccugucccugagugguggaggagccugaaccggggcuguggcuggagaguacgcugcuggucauacucacagcauucuucucaguagagcugggcacugaacuucucuggguagccgaagucuccuuuucuccaccugggguagagcuugcaugaccagaaccuguaacaacuguaagcacugugaggagcagcagcaggaagaaaggagacugggugcccggugucau

SEQ ID NO: 76 - RNA, 40 nt, siRNA
cucauaggggcuacgaucgguacugcuagggggcacauag

SEQ ID NO: 77 - RNA, 40 nt, siRNA
cagcugcccguaguucuuucggcggcacugacagacagcc

SEQ ID NO: 78 - RNA, 40 nt, siRNA
agacaaugccagcgcaaccagaacacagaccagcaccagc

SEQ ID NO: 79 - RNA, 40 nt, siRNA
gccuggcaccccagccccagacugggcagagaaaggaaau

SEQ ID NO: 80 - RNA, 40 nt, siRNA
gcugacgucugagaucgucagguuauaucgagaggcugcu

SEQ ID NO: 81 - RNA, 40 nt, siRNA
auugaacugugucuccacgucguggacauugaugguaccu

SEQ ID NO: 82 - RNA, 40 nt, siRNA
agucaauuguaccaccacagauccuggccugaacuuaaua

SEQ ID NO: 83 - RNA, 40 nt, RNA
aaaacccccuuguuuauaaaucugcaaaaacauuucagaa

SEQ ID NO: 84 - RNA, 40 nt, RNA
cucuugguaguagucggugcugggaucuuccagagaggaa

SEQ ID NO: 85 - RNA, 40 nt, RNA
ugaaaugugaaaagacaggaaaaagaaagagaccccagua

SEQ ID NO: 86 - RNA, 40 nt, RNA
agugcugugauuggaggaggugagaggagguaccgugcua

SEQ ID NO: 87 - RNA, 40 nt, RNA
ggcaucagucuuggugcuauggcuggcaagggugguagga

SEQ ID NO: 88 - RNA, 40 nt, RNA
agaggugccguugugcaccagaguagaagcugagccugau

SEQ ID NO: 89 - RNA, 40 nt, RNA
cagggcuggccuggugacugggaccgaggugacauccugu

SEQ ID NO: 90 - RNA, 40 nt, RNA
cacagcauucuucucaguagagcugggcacugaacuucuc

SEQ ID NO: 91 - RNA, 40 nt, RNA
cgcucauaggaugguagguaucccgggcuggaaagauguc

SEQ ID NO: 92 - RNA, 40 nt, RNA
ucuuucggcggcacugacagacagccaaggcaaaugagau

SEQ ID NO: 93 - RNA, 40 nt, RNA
caaccagaacacagaccagcaccagcagcgcgaugcccca

SEQ ID NO: 94 - RNA, 40 nt, RNA
ccccagacugggcagagaaaggaaauggcacaucacucac

SEQ ID NO: 95 - RNA, 40 nt, RNA
ucgucagguuauaucgagaggcugcuuccguuuuauacug

SEQ ID NO: 96 - RNA, 40 nt, RNA
ccacgucguggacauugaugguaccuucucggaaggccag

SEQ ID NO: 97 - RNA, 40 nt, RNA
ccacagauccuggccugaacuuaauauuggagaggcccag

SEQ ID NO: 98 - RNA, 40 nt, RNA
uauaaaucugcaaaaacauuucagaaaugucucucugcag

SEQ ID NO: 99 - RNA, 40 nt, RNA
cggugcugggaucuuccagagaggaauuaaacuggagguu

SEQ ID NO: 100 - RNA, 40 nt, RNA
aggaggugagaggagguaccgugcuauggugagugcuacu

SEQ ID NO: 101 - RNA, 40 nt, RNA
ugcuauggcuggcaagggugguaggaguaucagaguggug

SEQ ID NO: 102 - RNA, 40 nt, RNA
gcaccagaguagaagcugagccugaugcagagccugaggc

SEQ ID NO: 103 - RNA, 40 nt, RNA
ugacugggaccgaggugacauccugucccagguggcagcu

SEQ ID NO: 104 - RNA, 40 nt, RNA
caguagagcugggcacugaacuucucuggguagccgaagu

SEQ ID NO: 105 - protein, 164 aa, Homo Sapien
MTPGTQSPFFLLLLLTVLTATTAPKPATVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAG

SEQ ID NO: 106 - protein, 102 aa, Homo Sapien
FNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAG

SEQ ID NO: 107 - protein, 19 aa, ARTIFICIAL
  SYNTHESIZED SEQUENCE
FNSSLEDPSTDYYQELQRD

SEQ ID NO: 108 - protein, 100 aa, Homo Sapien
SSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAG

SEQ ID NO: 109 - protein, 20 aa, ARTIFICIAL
  SYNTHETIC SEQUENCE
SNIKFRPGSVVVQLTLAFRE

SEQ ID NO: 110 - DNA, 56 bp, ARTIFICIAL
  SYNTHESIZED SEQUENCE
tcgagggtaccatcaatgtccacggagtactgcgtggacattgatggtaccttttt

SEQ ID NO: 111 - DNA, 56 bp, ARTIFICIAL
  SYNTHESIZED SEQUENCE
ctagaaaaaggtaccatcaatgtccacgcagtactccgtggacattgatggtaccc

(19) United States
(12) Patent Application Publication
(10) Pub. No.: US 2005/0042209 A1
(43) Pub. Date: Feb. 24, 2005

(54) MUC1 extracellular domain and cancer treatment compositions and methods derived therefrom

(76) Inventors: Donald W. Kufe, Wellesley, MA (US); Tsuneya Ohno, Tokyo (JP)

Correspondence Address: ILEX ONCOLOGY, INC.;ATTN: FRANCES WINKLER, 4545 HORIZON HILL BLVD., SAN ANTONIO, TX, 78229, US

(21) Appl. No.: 10/778,859

(22) Filed: Feb. 13, 2004

Related U.S. Application Data
(63) Continuation-in-part of application No. 10/447,839, filed on May 29, 2003.
Application No. 10/447,839 is a continuation-in-part of application No. 10/293,391, filed on Nov. 13, 2002, now abandoned.
Application No. 10/293,391 is a continuation-in-part of application No. 09/951,938, filed on Sep. 11, 2001.
(60) Provisional application No. 60/231,841, filed on Sep. 11, 2000.

(51) Int. Cl. A61K 48/00

(52) U.S. Cl. 424/93.21; 514/44

(57) ABSTRACT

The present invention provides compositions and methods for increasing the sensitivity of cancer cells to chemotherapeutic agents by antagonists of MUC1 expression and/or activity.

This application is a continuation-in-part of U.S. patent application Ser. No. 10/447,839, filed May 29, 2003, which is continuation-in-part of U.S. patent application Ser. No. 10/293,391, filed Nov. 13, 2002, which is a continuation-in-part of U.S. patent application Ser. No. 09/951,938, filed Sep. 11, 2001, which claims priority to Provisional Application Ser. No. 60/231,841, filed Sep. 11, 2000.

The United States government may own rights in the present invention pursuant to grant number R21-CA87421, CA29431 and CA97098 from the National Cancer Institute, National Institutes of Health, Department of Health and Human Services.

### FIELD OF THE INVENTION

The present invention relates generally to the field of cancer therapy and more specifically to the use of modulators or agents that interact with MUC1 as a point on intervention in cancer therapy.

### BACKGROUND OF THE INVENTION

The human MUC1 mucin glycoprotein is expressed on the apical borders of secretory epithelial cells on the luminal surface of most glandular epithelia (Kufe et al., 1984). In carcinomas, MUC1 is highly overexpressed throughout the entire cell membrane and cytoplasm (Kufe et al., 1984; Perey et al., 1992). As such, the aberrant pattern of MUC1 expression in carcinoma cells may confer a function for MUC1 normally found at the apical membrane to the entire cell membrane. The hallmark of MUC1 mucin is an ectodomain comprising a glycosylated 20 amino acid extracellular sequence that is tandemly repeated 25-100 times in each molecule (Strouss & Decker, 1992). The mucin glycosylation level appears to be lower in cancer cells than normal cells of ductal epithelial tissue (Kufe, U.S. Pat. No. 5,506,343). This hypoglycosylation results in the exposure of tumor-specific epitopes that are hidden in the fully glycosylated mucin.

Over ninety percent of breast cancers show an increased expression of MUC1 (also known as Mucin, Epithelial Membrane Antigen, Polymorphic Epithelial Mucin, Human Milk Fat Globule Membrane antigen, Episialin, DF-3, etc., see Barry & Sharkey, 1985). Several clinical studies have suggested that mucinous tumor antigens expressed on the cell surface of tumor cells associate with poor prognosis of a variety of cancer types (Itzkowitz et al., 1990).

MUC1 is expressed as both a transmembrane form and a secreted form (Finn et al., 1995). The repeating sialyl epitopes of MUC1 (the “ectodomain”) are shed into the serum (Reddish et al., 1996). The N-terminal ectodomain (the extracellular domain that is cleaved, or “N-ter”) of MUC1 consists of a variable number of the 20-amino acid tandem repeats that are subject to O-glycosylation. This mucin extends far above the cell surface and past the glycocalyx making it easily available for interactions with other cells. The C-terminal region (“C-ter”) of MUC1 includes a 28 amino acid transmembrane domain and a 72 amino acid cytoplasmic tail that contains sites for tyrosine phosphorylation. An approximately 58-amino acid extracellular domain remains following cleavage of the ectodomain. It is not known what enzyme is responsible for the cleavage of the ectodomain at this time. The extracellular domain or “MUC1/ECD,” remaining, after cleavage of the ectodomain, typically includes the amino acid sequence:

TINVHDVETQFNQYKTEAASRYNLTISDVSVSDVPFPFSAQSGAG.

The cytoplasmic domain of MUC1 (“MUC1/CD”) encompasses multiple sub-domains that are important in intracellular signaling in cancer cells. β-Catenin binds directly to MUC1/CD at a SAGNGGSSL motif (Yamamoto et al., 1997). β-Catenin, a component of the adherent junctions of mammalian epithelium, binds to cadherins at the intracellular surface of the plasma membrane and performs a signaling role in the cytoplasm as the penultimate downstream mediator of the wnt signaling pathway (Takeichi, 1990; Novak & Dedhar, 1999). The ultimate mediator of the wnt pathway is a nuclear complex of β-catenin and lymphoid enhancer factor/T cell factor (Lef/Tcf), which stimulates the transcription of a variety of target genes (see e.g., Molenaar et al., 1996; Brunner et al., 1997). Defects in the β-catenin-Lef/Tcf pathway are involved in the development of several types of cancers (Novak & Dedhar, 1999).

Glycogen synthase kinase 3β (GSK3β) also binds directly to MUC1/CD and phosphorylates serine in a DRSPY site adjacent to the β-catenin binding motif, thereby decreasing the association between MUC1 and β-catenin (Li et al., 1998). In addition, the c-Src tyrosine kinase also binds to and phosphorylates a MUC1/CD SPYEKV motif, resulting in an increased interaction between MUC1/CD and β-catenin and a decreased interaction between MUC1/CD and GSK3β (Li et al., 2001).

MUC1 associates also constitutively with the epidermal growth factor receptor (EGF-R, HER1) at the cell membrane and activated EGF-R induces phosphorylation of the MUC1/CD SPYKEV motif (Li et al., 2001(a)). EGF-R mediated phosphorylation of MUC1/CD appears to increase the interaction of MUC1 with c-Src and β-catenin and downregulate the interaction between MUC1 and GSK3β. These results support a model wherein MUC1 integrates the signaling among c-Src, β-catenin and GSK3β pathways and dysregulation of this integrated signaling by aberrant overexpression of MUC1 in cancer cells could promote the transformed phenotype (Li et al., 2001(a)).

The Armadillo protein p120^{ctn} also binds directly to MUC1/CD resulting in the nuclear localization of p120 (Li & Kufe, 2001). P120 has been implicated in cell transformation and altered patterns of p120 expression have been observed in carcinomas (see e.g., Jawhari et al., 1999; Shimazui et al., 1996). P120 is a v-Src tyrosine kinase substrate, binds to E-cadherin, and is implicated as a transcriptional coactivator (Reynolds et al., 1989; Reynolds et al., 1994; Daniels & Reynolds, 1999). The observations that p120 localizes to both cell junctions and the nucleus has supported a role for p120, like β-catenin, in the regulation of both cell adhesion and gene transcription. Decreased cell adhesion resulting from association of MUC1 and p120 may be involved in increased metastatic potential of MUC1-expressing tumor cells.

Thus the available evidence indicates that MUC1/CD functions to transfer signals from the extracellular domain to the nucleus, and utilizes signaling mechanisms that have been implicated in adhesion receptor and growth factor signaling and cellular transformation. It is desirable to identify compositions and methods related to modulation of the MUC1-mediated signaling and its putative role in cellular transformation.

### SUMMARY OF THE INVENTION

The present invention encompasses methods of use and pharmaceutical compositions relating to the discovery that the extracellular domain of MUC1 provides binding domains for endogenous ligands and that such binding is related to an oncogenic function of MUC1 and the proliferation of cancer cells.

Broadly the invention relates to cancer treatment compositions and methods employing agents or treatment methodologies that comprise or include antagonists of MUC1 modulated cell proliferation. Preferred are methods and compositions that comprise agents that bind to MUC1/ECD, bind to MUC1/ECD ligands that activate the oncogenic function of MUC1, or downregulate the expression of MUC1.

Thus, one aspect of the present invention provides for a method for inhibiting the proliferation of cancer cells, comprising administration of an effective amount of a MUC1/ECD antagonist. MUC1/ECD antagonists are agents that downregulate or reduce the quantity of MUC1/ECD presented on cell surfaces, or downregulate the level of wild-type MUC1/ECD ligands available for binding to MUC1/ECD, and/or MUC1/ECD binding inhibitors. A “MUC1/ECD binding inhibitor” means a compound that inhibits the binding of MUC1 wild-type ligands, which may suitably include neuregulin 2 isoform 5 (SEQ ID NO: 2), neuregulin 2 isoform 6 (SEQ ID NO: 3), and appropriate fragments thereof, to MUC1/ECD or a compound that inhibits the binding of an antibody that binds to an epitope within SEQ ID NO. 4 to MUC1/ECD. Appropriate fragments of neuregulin 2 isoform 5 (SEQ ID NO: 2) and neuregulin 2 isoform 6 (SEQ ID NO: 3) are those that bind to MUC1/ECD. MUC1/ECD binding inhibitors include antibodies, polypeptides and small molecules that inhibit such binding. A “MUC1/ECD-P1 binding inhibitor” means a MUC1/ECD binding inhibitor identified by inhibition of the binding to MUC1/ECD of an antibody the binds to an epitope within SEQ ID NO. 4.

In one embodiment of the invention, the MUC1/ECD inhibitor is the polypeptide of SEQ ID NO: 1, or a fragment comprising at least four consecutive amino acids of SEQ ID. NO: 1 such as TINV, NVHD, VHDV, DVET, VETQ, ETQF, TQFN, QFNQ, FNQY, NQYK, QYKT, YKTE, KTEA, TEAA, EAAS, AASR, ASRY, SRYN, RYNL, YNLT, NLTI, LTIS, TISD, ISDV, SDVS, DVSV, VSVS, SVSD, VSDV, SDVP, DVPF, VPFP, PFPF, FPFS, PFSA, FSAQ, SAQS, AQSG, QSGA, and SGAG. In other embodiments, the MUC1/ECD inhibitor is a conservative variant of the foregoing peptides. In another embodiment, the MUC1/ECD binding inhibitor is the polypeptide of SEQ ID NO: 4, SEQ ID NO: 5, or conservative variants thereof.

In another embodiment of the present invention, the MUC1/ECD inhibitor is an antibody that binds to one or more epitopes in the MUC1/ECD sequence SEQ ID NO: 1. In other embodiments of the invention, the MUC1/ECD inhibitor is an antibody that binds to an epitope within SEQ ID NO: 2 or SEQ ID NO: 3. The antibody may be a polyclonal or a monoclonal antibody. Monoclonal antibodies may be humanized or human monoclonal antibodies. It may also be a bispecific antibody or a fragment which comprises an antigen binding region. In some embodiments, the antibody is conjugated to a chemotherapeutic agent, radioisotope, toxin, or an effector that induces a cytolytic or cytotoxic immune response. Such conjugates may comprise a cytokine, an antimetabolite, an anthracycline, a vinca alkaloid, an antibiotic, an alkylating agent, a naturally derived toxin, or an Fc region of a IgG1 immunoglobulin.

In another embodiment, the method further comprises the administration of a chemotherapeutic agent or radiation in combination with a MUC1/ECD antagonist. Chemotherapeutic agents typically include alkylating agents, topoisomerase inhibitors, antimetabolites, tubulin interactive agents, anti-hormonal agents, ornithine decarboxylase inhibitors and tyrosine kinase inhibitors.

In various embodiments, the cancer cells are selected from the group consisting of skin cancer cells, prostate cancer cells, lung cancer cells, brain cancer cells, breast cancer cells, ovarian cancer cells, cervical cancer cells, liver cancer cells, pancreatic cancer cells, colon cancer cells, stomach cancer cells and leukemia cells.

Another aspect of the invention is a method for reducing tumor growth in a mammal comprising administration of a therapeutic amount of a chemotherapeutic agent or radiation and an effective amount of a MUC1/ECD antagonist. In a preferred embodiment, the mammal is human. In one embodiment the method is for treating refractory tumors comprising administration of a therapeutic amount of a chemotherapeutic agent or radiation and an effective amount of a MUC1/ECD antagonist subsequent to treatment with one or more chemotherapeutic agents. In various embodiments, the tumor is a tumor of the skin, prostate, lung, brain, breast, ovary, cervix, liver, pancreas, colon, stomach or heampoietic system.

Other aspects of the invention relate to pharmaceutical compositions comprising MUC1/ECD antagonists and a pharmaceutically acceptable carrier. Wherein the antagonist is a MUC1/ECD binding inhibitor that may be the polypeptide of SEQ ID NO: 1 or a fragment comprising at least four consecutive amino acids, or conservative variants thereof, and a pharmaceutically acceptable carrier. In some embodiments, the MUC1/ECD inhibitor may be the polypeptide of SEQ ID NO: 4, SEQ ID NO: 5, or conservative variants thereof. In other embodiments, the pharmaceutical composition comprises an antibody that is a MUC1/ECD binding inhibitor and binds to an epitope within sequences of the peptides selected from the group consisting of SEQ ID NO: 1, SEQ ID NO: 2 and SEQ ID NO: 3 and a pharmaceutically acceptable carrier.

One aspect of the present invention is a method of enhancing the induction apoptosis in a MUC1 expressing cancer cell by a pro-apoptotic chemotherapeutic agent, comprising first contacting the cancer cell with an effective amount of a MUC1/ECD antagonist and subsequently contacting the cancer cell with an effective amount of a pro-apoptotic chemotherapeutic agent. A pro-apoptotic chemotherapeutic agent is a chemotherapeutic agent that decreases cancer cell proliferation and/or viability by, at least in part, inducing apoptosis. The pro-apoptotic chemotherapeutic agent may be a DNA-interactive chemotherapeutic agent, a tubulin interactive chemotherapeutic agent, or an antimetabolite chemotherapeutic agent. The MUC1/ECD antagonist may be an agent that downregulates MUC1, such agents including interference RNA molecules and small molecules.

Another aspect of the invention is a method of decreasing the proliferation of a cancer cell comprising: (a) identifying the cancer cell as a MUC1 expressing cancer cell; (b) contacting the MUC1 expressing cancer cell with an effective amount of a MUC1/ECD antagonist; and (c) subsequently contacting the MUC1 expressing cancer cell with an effective amount of a pro-apoptotic chemotherapeutic agent.

Another aspect of the invention is a method of killing a MUC1 expressing cancer cell, wherein said MUC1 expressing cancer has been subjected to a first pro-apoptotic chemotherapeutic agent, comprising: (a) contacting the MUC1 expressing cancer cell with an effective amount of a MUC1/ECD antagonist; and (b) subsequently contacting the MUC1 expressing cancer cell with an effective amount of a second pro-apoptotic chemotherapeutic agent. The first and second pro-apoptotic chemotherapeutic agents may be the same or different and may be DNA interactive chemotherapeutic agents, tubulin interactive chemotherapeutic agents, or antimetabolite chemotherapeutic agents.

The present invention also encompasses methods for screening MUC1/ECD binding inhibitor activity. One embodiment comprises a method of identifying a compound that inhibits the binding of ligands to MUC1/ECD, the method comprising: (a) providing a polypeptide comprising SEQ ID. NO: 1 or SEQ ID NO: 5; (b) contacting said polypeptide with a test compound and a ligand to the extracellular domain of MUC1 selected from the group consisting of antibodies to MUC1/ECD that stimulate MUC1 mediated cancer cell proliferation and wild type ligands that bind to MUC1/ECD and stimulate cancer cell proliferation; and (c) determining whether the binding of said antibody to MUC1/ECD or wild type ligand is decreased relative to an appropriate control. Appropriate controls include, but are not limited to, assays wherein test compounds are excluded. In one embodiment the MUC1/ECD antibody that stimulates MUC1 mediated cancer cell proliferation is an antibody that binds to an epitope within SEQ ID NO. 4. In other embodiments, the wild type ligands may suitable include neuregulin 2 isoform 5 (SEQ ID NO: 2) and appropriate fragments thereof and neuregulin 2 isoform 6 (SEQ ID NO: 3) and appropriate fragments thereof, wherein appropriate fragments are those that bind to the SEQ ID NO: 1 and have suitable growth stimutatory activity.

Another embodiment is a method of identifying a compound that inhibits the proliferation of MUC1-expressing cancer cells, the method comprising: (a) providing a population of MUC1-expressing cancer cells; (b) contacting said population of MUC1-expressing cancer cells with a test compound and a ligand to the extracellular domain of MUC1 selected from the group consisting of antibodies to MUC1/ECD that stimulate MUC1 mediated cancer cell proliferation and wild type ligands that bind to MUC1/ECD and stimulate cancer cell proliferation; and (c) determining whether the proliferation of the population of MUC1-expressing cancer cells is decreased by comparison to an appropriate control. Appropriate controls include, but are not limited to, proliferation assays wherein test compounds are excluded. In one embodiment the MUC1/ECD antibody that stimulates MUC1 mediated cancer cell proliferation is an antibody that binds to an epitope within SEQ ID NO. 4. In other embodiments, the wild type ligands may suitable include neuregulin 2 isoform 5 (SEQ ID NO: 2) and appropriate fragments thereof and neuregulin 2 isoform 6 (SEQ ID NO: 3) and appropriate fragments thereof, wherein appropriate fragments are those that bind to the SEQ ID NO: 1 and have suitable growth stimulatory activity.

The present invention also provides methods for identifying compounds that downregulate MUC1/ECD expression. The method comprises: (a) providing a population of MUC1-expressing cancer cells; (b) contacting said population of MUC1-expressing cancer cells with a test compound; (c) utilizing an anti-MUC1/ECD antibody to identify polypeptides comprising MUC1/ECD in the MUC1-expressing cancer cells; and (d) determining whether the expression of polypeptides comprising MUC1/ECD is decreased in comparison to controls wherein the test compound was excluded.

The present invention also encompasses pharmaceutical compositions comprising compounds identified by the foregoing methods and a pharmaceutically acceptable carrier.

Further aspects of the present invention provide for interfering RNA compositions that can downregulate MUC1 expression and methods of use of such compositions. One aspect provides for a double stranded RNA complex comprising a first RNA sequence of 19 to 23 nucteotides that will hybridize to SEQ ID NO: 19 under stringent conditions, or in a preferred embodiment will hybridize to SEQ ID NO: 20 under stringent conditions, and a second RNA sequence of 19 to 23 nucteotides that wilt hybridize to the first RNA under stringent conditions. The first and second RNA sequences may be separate RNA oligonucleotides or may be two portions of a single RNA oligonucteotide. In some embodiments, the double stranded RNA complex may comprise at least one modified internucleoside linkage and/or at least one modified sugar moiety.

Another aspect of the invention provides for a 5′ phosphorylated RNA otigonucteotide of 29 to 40 bases that will hybridize under stringent conditions to SEQ ID NO: 19, or in a preferred embodiment will hybridize to SEQ ID NO: 20. In various embodiments, the 5′ phosphorylated RNA oligonucteotide of may comprise at least one modified internucteoside linkage and/or at least one modified sugar moiety.

The present invention also provides for a method of inhibiting the expression of MUC1, comprising contacting a cell that expresses MUC1 with an interfering RNA oligonucteotide, that wilt hybridize with SEQ ID NO: 19 under stringent conditions, or in a preferred embodiment, wilt hybridize with SEQ ID NO: 20 under stringent conditions. In some embodiments, the interfering RNA oligonucleotide is a first RNA sequence of 19 to 23 nucleotides of a double stranded RNA complex comprising a second RNA sequence of 19 to 23 nucleotides, wherein the first RNA sequence will hybridize to the second RNA sequence under stringent conditions. The first and second RNA sequences may be separate RNA oligonucleotides or they may be two portions of a single RNA oligonucteotide. The double stranded RNA complex may comprise at least one modified internucleoside linkage and/or at least one modified sugar moiety. In other embodiments, the interfering RNA oligonucleotide is a 5′ phosphorylated RNA oligonucleotide of 29 to 40 bases that may comprise at least one modified internucleoside linkage and/or at least one modified sugar moiety. In some embodiments, the MUC1 expressing cell is a cancer cell which in various embodiments may be a skin cancer cell, a prostate cancer cell, a lung cancer cell, a brain cancer cell, a breast cancer cell, an ovarian cancer cell, a cervical cancer cell, a liver cancer cell, a pancreatic cancer cell, a colon cancer cell, a stomach cancer cell or a leukemia cell.

Another aspect of the invention is a method of inhibiting the proliferation of a cancer cell that expresses MUC1 comprising contacting the cancer cell with an interfering RNA oligonucleotide, that will hybridize with SEQ ID NO: 19 under stringent conditions, or in a preferred embodiment, will hybridize with SEQ ID NO: 20 under stringent conditions. In some embodiments, the interfering RNA oligonucleotide is a first RNA sequence of 19 to 23 nucleotides of a double stranded RNA complex comprising a second RNA sequence of 19 to 23 nucleotides, wherein the first RNA sequence will hybridize to the second RNA sequence under stringent conditions. The first and second RNA sequences may be separate RNA oligonucleotides or they may be two portions of a single RNA oligonucleotide. The double stranded RNA complex may comprise at least one modified internucleoside linkage and/or at least one modified sugar moiety. In other embodiments, the interfering RNA oligonucleotide is a 5′ phosphorylated RNA oligonucleotide of 29 to 40 bases that may comprise at least one modified internucleoside linkage and/or at least one modified sugar moiety. In various embodiments, the cancer cell is a skin cancer cell, a prostate cancer cell, a lung cancer cell, a brain cancer cell, a breast cancer cell, an ovarian cancer cell, a cervical cancer cell, a liver cancer cell, a pancreatic cancer cell, a colon cancer cell, a stomach cancer cell or a leukemia cell.

The present invention further provides for an interfering RNA composition comprising an RNA oligonucleotide of about 17 to about 50 bases that can inhibit the expression of MUC1 when administered to the cell in an effective amount.

Further aspects of the present invention provide for methods of inhibiting the expression MUC1 in a cell, which may be a cancer cell, comprising administering an interfering RNA oligonucleotide of about 17 to about 50 bases in length in an amount effective to inhibit the expression of MUC1. Also provided are methods of inhibiting the proliferation of a MUC1 expressing cancer cell, comprising administering an interfering RNA oligonucleotide of about 17 to about 50 bases in length in an amount effective to inhibit the proliferation of the cancer cell.

The present invention also encompasses pharmaceutical compositions comprising interfering RNA of the present invention and a pharmaceutically acceptable carrier.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Effect of anti-MUC1-P1 antibody on proliferation of ZR-75-1 breast carcinoma cells.

FIG. 2: Effect of anti-MUC1-P1 antibody on proliferation of SW480 cells stably expressing an empty vector (SW480/V) or MUC1(SW480/MUC1).

FIG. 3: Effect of ZR-75-1 conditioned medium on proliferation of SW480 cells stably expressing an empty vector (SW480/V) or MUC1(SW480/MUC1).

FIG. 4: Effect of MUC1 on H₂O₂ and taxol-induced apoptosis in HeLa cells stably expressing an empty vector (HeLa/V) or MUC1(HeLa/Muc1). The results are expressed as the percentage apoptosis (mean±SE) of three separate experiments.

FIG. 5: Effect of anti-MUC1 siRNAs #1 and #2. FIGS. 5A and 5B show the decrease in MUC1 protein expression elicited by siRNAs as shown by Western blot analysis in A549 and MCF-7 cells respectively. FIGS. 5C and 5D show the decrease in MUC1 RNA elicited by siRNAs as shown by RT-PCR immunoblot in A549 and MCF-7 cells respectively.

FIG. 6: Effect of anti-MUC1 siRNAs #3, #4 and #5 on MUC1 protein expression in MCF-7 cells as shown by immunoblot.

FIG. 7: Effect on apoptosis in A549 cells of the combination of transfection of siRNA #2 plus treatment with cisplatinum (CDDP).

FIG. 8: Effect on proliferation in A549 cells of the combination of transfection of siRNA #1 or siRNA #2 or plus treatment with cisplatinum (CDDP).

FIG. 9: Depiction of plasmid (pU6-MUC1siRNA #2) that expresses siRNA #2.

FIG. 10: Binding of antibody IPB-01 to immobilized MUC1-Yex-mFc.

FIG. 11: Binding of antibody IPB-02 to immobilized MUC1 -Yex-mFc.

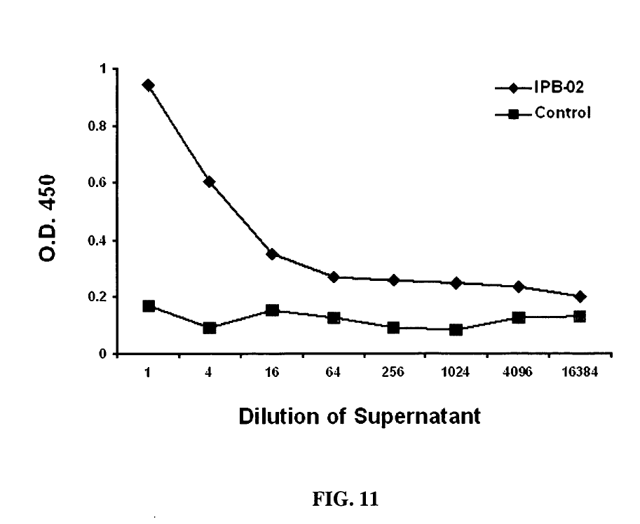

FIG. 12: Summary of data showing that MUC1 confers resistance to CDDP in vivo. ZR-75-1/vector cells (∘, ●) or ZR-75-1/MUC1siRNA (¤, ▪) cells (1×10⁷) were injected into nude mice that had been pretreated with β-estradiol. The mice were treated as indicated (arrows) with intraperitoneal injections of PBS (¤, ∘) or 7 mg/Kg CDDP (●, ▪). The results are expressed as the tumor volume (mean±S.D>of 4-8 mice per group.

FIG. 13: Depiction of SDS-PAGE and immunoblotting with indicated antibodies of mitochondrial fractions from HCT116/vector-A, HCT116/MUC1-A and HCT116/MUC1(Y46F)-A cells.

FIG. 14: Depiction of SDS-PAGE and immunoblotting with indicated antibodies of mitochondrial fractions from HCT116/MUC1-A and HCT116/MUC1(Y46F)-A cells that had been treated with heregulin (HRG) for the indicated times.

FIG. 15: Summary of ciplatin (CDDP) induced apoptosis in HCT116/vector-A, HCT116/MUC1-A and HCT116/MUC1(Y46F)-A cells wherein cells were incubated with 100 μM CDDP for 24 hr then analyzed for sub-G1 DNA.

FIG. 16: Summary of apoptosis induced in both A and B clones of HCT116/vector, HCT116/MUC1 and HCT116/MUC1(Y46F) cells when left untreated (open bars) or treated with 100 μM CDDP for 24 hr (solid bars). The results are presented as percentage apoptosis (mean±SD of three independent experiments) as determined by analysis of sub-G1 DNA.

FIG. 17: Summary of apoptosis induced in both A and B clones of HCT116/vector, HCT116/MUC1 and HCT116/MUC1(Y46F) cells when left untreated (open bars) or treated with 70 μM etoposide for 48 hr (solid bars). The results are presented as percentage apoptosis (mean±SD of three independent experiments) as determined by analysis of sub-G1 DNA.

FIG. 18: Summary of apoptosis induced in both A and B clones of HCT116/vector, HCT116/MUC1 and HCT116/MUC1(Y46F) cells when left untreated (open bars) or treated with 20 ng/mt TNF-α and 10 ng/ml cyctohexamide (CHX) for 12 hr (solid bars). The results are presented as percentage apoptosis (mean±SD of three independent experiments) as determined by analysis of sub-G1 DNA.

FIG. 19: Summary in left panel of apoptosis induced in HCT116/vector-A, HCT116/MUC1-A and HCT116/MUC1(Y46F)-A cells when left untreated (open bars) or treated with 100 ng/mt TRAIL for 14 hr (closed bars). Summary in right panel of apoptosis induced in HCT116/MUC1(Y46F)-A cells when treated with 100 ng/ml TRAIL and/or 10 μM CHX as indicated for 14 hr. The results are presented as percentage apoptosis (mean±SD of three independent experiments) as determined by analysis of sub-G1 DNA.

### DETAILED DESCRIPTION OF THE INVENTION

I. Polypeptides

The polypeptides of the present invention can be created by synthetic techniques or recombinant techniques which employ genomic or cDNA cloning methods.

Polypeptides can be routinely synthesized using solid phase or solution phase peptide synthesis. Methods of preparing relatively short polypeptides peptides, such as P0 (SEQ ID NO: 9), P1 (SEQ ID NO: 4), P2 (SEQ ID NO: 6) and P3 (SEQ ID NO: 7), by chemical synthesis are well known in the art. Such polypeptides could, for example be produced by solid-phase peptide synthesis techniques using commercially available equipment and reagents such as those available from Milligen (Bedford, Mass.) or Applied Biosystems-Perkin Elmer (Foster City, Calif.). Alternatively, segments of such polypeptides could be prepared by solid-phase synthesis and linked together using segment condensation methods such as those described by Dawson et al., (1994). During chemical synthesis of such polypeptides, substitution of any amino acid is achieved simply by replacement of the residue that is to be substituted with a different amino acid monomer.

Wild-type MUC1/ECD ligand polypeptides can be identified as exemplified in Example 3 herein. Recombinant MUC1/ECD ligands can then be prepared by methods known in the art.

The polypeptides of the present invention include variant polypeptides. By “variant” polypeptide is intended a polypeptide sequence modified by deletion or addition of one or more amino acids at one or more sites in the sequence; or substitution of one or more amino acids at one or more sites within the sequence. Variant polypeptides encompassed by the present invention retain the desired biological activity of the polypeptide from which they are derived. Such variants will have at least 40%, 50%, 60%, 70%, generally at least 75%, 80%, 85%, preferably about 90% to 95% or more, and more preferably about 98% or more sequence identity to the amino acid sequence of the polypeptide from which they are derived. The percentage of sequence identity, also termed homology, between a polypeptide native and a variant sequence may be determined by comparing the two sequences using the Gap program (Wisconsin Sequence Analysis Package, Version 8 for Unix, Genetics Computer Group, University Research Park, Madison Wis.), which uses the algorithm of Smith and Waterman, (1981).

The polypeptides of the present invention also include variant polypeptides with one or more conservative substitutions. For the purposes of classifying amino acid substitutions as conservative, amino acids are grouped as follows: Group I (hydrophobic sidechains): norleucine, met, ala, val, leu, ile; Group II (neutral hydrophilic side chains): cys, ser, thr; Group III (acidic side chains): asp, glu; Group IV (basic side chains): asn, gin, his, lys, arg; Group V (residues influencing chain orientation): gly, pro; and Group VI (aromatic side chains): trp, tyr, phe. Conservative substitutions involve substitutions between amino acids in the same class.

Also encompassed by the present invention are chemical derivatives of polypeptides. “Chemical derivative” refers to a subject polypeptide having one or more residues chemically derivatized by reaction of a functional side group. Such derivatized residues include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboyl groups may be derivatized to form salts, methyl and ethyl esters or other types of esters or hyrazides. Free hydroxyl groups may be derivatized to form O-acyl or O-alkyl derivatives. The imadazole group of histidine may be derivatized to form N-imbenzylhistidine.

The term “polypeptide” as used herein indicates a molecular chain of amino acids and does not refer to a specific length of the product.

II. Antibodies

The term “antibody” is used in the broadest sense and specifically covers monoclonal antibodies (including full length monoclonal antibodies), polyclonal antibodies, multispecific antibodies (e.g., bispecific antibodies), and antibody fragments, so long as they exhibit the desired biological activity.

Methods for generating polyclonal antibodies are well known in the art. Briefly, a polyclonal antibody is prepared by immunizing an animal with an antigenic composition and collecting antisera from that immunized animal. A wide range of animal species can be used for the production of antisera including rabbit, mouse, rat, hamster, guinea pig and goat.

As is well known in the art, a given composition may vary in its immunogenicity. It is often necessary therefore to boost the host immune system, as may be achieved by coupling a polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine. As is also well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The serum for an immunized animal may be used as is for various applications or the desired antibody fraction may be purified by well-known methods, such as affinity chromatography using another antibody or a peptide bound to a solid matrix.

Monoclonal antibodies (MAbs) may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified expressed polypeptide. The immunizing composition is administered in a manner that effectively stimulates antibody producing cells.

The methods for generating monoclonal antibodies (MAbs) generally begin along the same lines as those for preparing polyclonal antibodies. The use of rats may provide certain advantages (Goding, 1986), but mice are preferred, with the BALB/c mouse being the most routinely used and generally gives a higher percentage of stable fusions.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred. Often, a panel of animals will have been immunized and the spleen of animal with the highest antibody titer will be removed and obtaining lymphocytes from the spleen.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and have enzyme deficiencies that render them incapable of growing in certain selective media that support the growth of only the desired fused cells (hybridomas). Selected hybridomas are serially diluted and cloned into individual antibody-producing cell lines, which can then be propagated indefinitely to provide MAbs.

In accordance with the present invention, fragments of the monoclonal antibody of the invention can be obtained from the monoclonal antibody produced as described above, by methods which include digestion with enzymes such as pepsin or papain and/or cleavage of disulfide bonds by chemical reduction. Alternatively, monoclonal antibody fragments encompassed by the present invention can be synthesized using an automated synthesizer, or by expression of full-length gene or of gene fragments in E. coli or other recombinant microorganisms and cell lines.

The present invention also encompasses various antibody conjugates. Conjugates with fluorescein markers are prepared be methods known in the art, such as conjugation in the presence of coupling agents or by reaction with an isothiocyanate. Conjugates with metal chelates are similarly produced. Other moieties to which antibodies may be conjugated include radionuclides such as ¹³¹I, ⁹⁰Y, ¹⁰⁵Rh, ⁴⁷Sc, ⁶⁷Cu, ²¹²Bi, ²¹¹At, ¹⁸⁸Re, ¹⁰⁹Pd, ⁴⁷Sc, ²¹²Pb, and ¹⁵³Sm and the like, as described in Gansow, 1991, which is herein incorporated by reference.

Monoclonal antibodies of the invention can also be coupled to conventional chemotherapeutic agents such as an antimetabolite, an anthracycline, a vinca alkaloid, an antibiotic or an alkylating agent. Drugs that may be coupled to the antibodies for targeting include compounds such as doxorubicin, cyclophosphamide, cisplatin, adriamycin, estramustine, fluorouracil, ethinyl estradiol, mitoxantrone, methotrexate, finasteride, taxol, and megestrol. Methods of coupling may be direct via covalent bonds, or indirect via linking molecules, and will generally be known in the art for the particular drug selected and are made using a variety of bifunctional protein coupling agents. Examples of such reagents are SPDP, IT, bifunctional derivatives of imidoesters such a dimethyl adipimidate HCl, active esters such as disuccinimidyl suberate, aldehydes such as glutaraldehyde, bisazido compounds such as his (R-azidobenzoyl)hexanediamine, bisdiazonium derivatives such as bis-(R-diazoniumbenzoyl)ethylenediamine, diisocyanates such as tolylene 2,6-diisocyanate, and bis-active fluorine compounds such as 1,5-difluoro-2,4-dinitrobenzene. (See, e.g., Thorpe et al., 1982, herein incorporated by reference).

The antibodies of the present invention may also be conjugated with various toxin molecules or an effector such as IgG1 immunoglobulin, which induces cytolytic or cytotoxic immune response. Thus, the two components may be chemically bonded together by any of a variety of well-known chemical procedures. For example, the linkage may be by way of heterobifunctional cross-linkers, e.g. SPDP, carbodiimide, glutaraldehyde, or the like. The toxin molecules may also be fused to the antibody or binding regions thereof by recombinant means, such as through the production of single chain antibodies. The genes encoding protein chains may be cloned in cDNA or in genomic form by any cloning procedure known to those skilled in the art (see e.g., Sambrook et al., 1989). The recombinant production of various immunotoxins is well-known within the art and can be found, for example in Thorpe et al., 1982(a), Waldmann, 1991, and Pastan et al., 1992, all herein incorporated by reference. A variety of toxin molecules are suitable for use as the cytotoxic domain in the antibody conjugates or fusion proteins described here. Any toxin known to be useful as the toxic component of an immunotoxin may be used, preferably a protein toxin that may be recombinantly expressed. Particularly useful as the cytotoxic domain are bacterial toxins such as Pseudomonas exotoxin A (PE), diphtheria toxin, shiga toxin and shiga-like toxin, and ribosome inactivating toxins derived from plants and fungi, including ricin, α-sarcin, restrictotocin, mitogellin, tricanthosin, saporin-G, saporin-1, momordin, gelonin, pokeweed antiviral protein, abrin, modeccin and others described in Genetically Engineered Toxins, ed. A. Frankel, Marcel Dekker, Inc., 1992, herein incorporated by reference, and any recombinant derivatives of those proteins (see Olsnes 1981; U.S. Pat. No. 4,675,382; and U.S. Pat. No. 4,894,443, herein incorporated by reference).

The antibody may also be a bispecific antibody which recognizes both the MUC1/ECD and an antigen which promotes the release of a cytokine such as IL-1, TNF alpha and CD16, CD2, CD3 L.C. CD28, which in turn, will activate the release of IFN gamma or TNF alpha, respectively.

The Mabs of the present invention encompass chimeric Mabs, including, “humanized” forms of non-human (e.g., murine) Mabs. Humanized MAbs are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. (see Jones et al., 1986; Riechmann et al., 1988; and Presta, 1992). Fully human MAbs are preferred in the therapeutic methods of the present invention.

“Single-chain FV” or “sFv” antibody fragments of the present invention comprise the VH and VL domains of antibody, wherein these domains are present in a single polypeptide chain. Generally, the Fv polypeptide further comprises a polypeptide linker between the VH and VL domains which enable the sFv to form the desired structure for antigen binding (see Pluckthun, 1994).

III. Screening and Diagnostic Assays

The present invention provides for methods for identifying compounds that inhibit the binding of various ligands to MUC1/ECD. The binding ligands include neuregulin 2 isoform 5 (SEQ ID NO: 2), neuregulin 2 isoform 6 (SEQ ID NO: 3) and fragments of either isoform that bind to MUC1/ECD and, in a preferred embodiment, an antibody that binds to an epitope within SEQ ID NO: 4.

In one embodiment, the screening method utilizes an in vitro competitive binding assay, wherein the capacity of a test compound to inhibit the binding of the aforementioned ligands to a polypeptide comprising SEQ ID NO.1 or SEQ ID NO: 5 is assessed. In such an assay, the polypeptide comprising MUC1/ECD derived sequences SEQ ID NO.1 or SEQ ID NO: 5 may be conjugated to another protein or produced as a fusion protein, e.g., the GST-MUC1/ECD fusion protein exemplified herein in Example 3. Other suitable conjugates and fusion proteins may be made by one of skill in the art utilizing procedures know in the art. The polypeptides or MUC1/ECD ligands may be labeled with a radioisotope or fluorescent label (e.g., phycobiliproteins, such as phycoerythrin and allophycocyanins, fluorescein and Texas red). Alternatively an enzyme, such as peroxidase, may be used and conjugated either directly or indirectly via a biotin and avidin or streptavidin system. Decreased binding upon introduction of a test compound is indicative of competitive binding.

A compound that inhibits the binding of ligands to MUC1/ECD may be a modulator that is an antagonist or agonist of the biological activity initiated by MUC1/ECD binding by neregulin 2 isoforms 5 or 6. E.g., the antibody raised to polypeptide P1 (SEQ ID. NO 4) is expected to inhibit binding of the wild-type ligands but acts as an agonist for the MUC1/ECD binding site, i.e., it stimulates proliferation of carcinoma cells. In contrast, appropriate compounds, such as the MUC1/ECD polypeptide SEQ ID NO. 1, will bind to the endogenous wild-type ligands thereby preventing binding to MUC1/ECD and consequently acting as an antagonist, i.e., preventing or decreasing the proliferation of carcinoma cells that would be otherwise observed upon binding of the MUC1/ECD ligands.

An alternative screening assay can discriminate between MUC1/ECD binding inhibitors that exhibit antagonist and agonist activity in regard to the proliferation of MUC1-expressing cancer cells. The method requires a population of MUC1-positive cancer cells, preferably human cancer cells. This could be a population of cells that constitutively expresses MUC1, but the population is preferably of a cell type engineered to express MUC1. The latter are more versatile in regard to providing cells for appropriate controls, e.g. cells engineered with an empty vector, and also for enabling the construction of cells expressing MUC1 mutants. Examples of engineered MUC1 cancer cells include, but are not limited to, SW480 and HCT116 colon cancer cells as exemplified in Examples 2 and 4 herein. Inhibition of MUC1/ECD ligand-induced cell proliferation will indicate a test compound with antagonist activity. Controls may comprise incubation of cancer cells engineered with an empty vector (i.e. MUC1-negative) or incubation of MUC1-positive cells in the absence of either the test compound or the MUC1/ECD ligand. One of the latter controls will identify agonists, i.e., stimulation of cancer cell proliferation observed in incubations in which the test compound is present and the MUC1/ECD ligand is absent. Specificity of the agonist activity is established by use of engineered MUC1-negative cells.

Yet another screening assay monitors MUC1/ECD ligand induced phosphorylation of the intracellular domain of MUC1. Alternate screening methodologies employ monitoring of MUC1/ECD ligand induced association of MUC1 with EGF-R, s-Src, β-catenin, GSK3β or p120. Methods for monitoring such phosphorylation and protein associations are described in Li et al., (1998), Li et al., (2001), Li et al., (2001(a)) and Li & Kufe, (2001), all herein incorporated by reference.

The present invention also provides for methods for identifying compounds that downregulate the expression of MUC1/ECD. In some embodiments of the invention, labeled antibodies to MUC1/ECD are utilized to visualize the expression of MUC/ECD in appropriate cell lines by flow cytometry or by immunohistochemistry, using methods know in the art. Alternatively, the expression of MUC1 can be estimated by immunoblotting or by probing total cellular RNA with labeled DNA probes, e.g., as described in Example 7 herein.

Estimation of the expression of MUC1/ECD can also be used for diagnostic methodologies, wherein antibodies to MUC1/ECD are utilized to investigated the expression of MUC1/ECD on or in cells derived from a subject. Such antibodies can also be utilized for imaging of cancer cells within a subject. Imaging is performed by labeling the anti-MUC1/ECD antibody, e.g., with a radiolabel, and injecting the antibody to a subject and monitoring the location of the antibody within the body of said subject.

IV. Combination with Chemotherapeutic Agents

The present invention encompasses the use of the MUC1/ECD antagonists and agents that downregulate the expression of MUC1, e.g., MUC1 antisense, RNAi and small molecules, in combination with chemotherapeutic agents. While not being limited by any particular theory, MUC1 inhibits the apoptotic response to genotoxic stress induced by certain chemotherapeutic agents, and thereby induces resistance to such agents. MUC1/ECD antagonists and agents that downregulate the expression of MUC1, may be used to mitigate this MUC1 mediated response to chemotherapeutic agents, thereby enhancing the effectiveness of such agents. In this regard, MUC1/ECD antagonists and agents that downregulate the expression of MUC1 will be useful for the treatment cancer cells resistant to chemotherapeutic agents, including residual cancers remaining or reoccurring after cancer chemotherapy. The foregoing rational also pertains to the combination of MUC1/ECD antagonists or agents that downregulate the expression of MUC1 and ionizing radiation.

The chemotherapeutic agents useful in the methods of the invention include the full spectrum of compositions and compounds which are known to be active in killing and/or inhibiting the growth of cancer cells. The chemotherapeutic agents, grouped by mechanism of action include DNA-interactive agents, antimetabolites, tubutin interactive agents, anti-hormonals, anti-virals, ODC inhibitors and other cytotoxics such as hydroxy-urea. Any of these agents are suitable for use in the methods of the present invention.

DNA-interactive agents (genotoxic agents) include the alkylating agents, e.g., cisplatin, cyclophosphamide, attretamine; the DNA strand-breakage agents, such as bleomycin; the intercalating topoisomerase II inhibitors, e.g., dactinomycin and doxorubicin); the nonintercalating topoisomerase II inhibitors such as, etoposide and teniposide; and the DNA minor groove binder plicamycin.

The alkylating agents form covalent chemical adducts with cellular DNA, RNA and protein molecules and with smaller amino acids, glutathione and similar chemicals. Generally, these alkylating agents react with a nucleophilic atom in a cellular constituent, such as an amino, carboxyl, phosphate, sulfhydryl group in nucleic acids, proteins, amino acids, or glutathione. The mechanism and the role of these alkylating agents in cancer therapy is not well understood. Typical alkylating agents include: nitrogen mustards, such as chlorambucil, cyclophosphamide, ifosfamide, mechlorethamine, melphalan, uracil mustard; aziridine such as thiotepa; methanesulphonate esters such as busulfan; nitroso ureas, such as carmustine, lomustine, streptozocin; platinum complexes such as cisplatin, carboplatin; bioreductive alkylators, such as mitomycin and procarbazine, dacarbazine and altretemine; DNA strand-breaking agents including bleomycin.

Topoisomerases are ubiquitous cellular enzymes which initiate transient DNA strand breaks during replication to allow for free rotation of the strands. The functionality of these enzymes is critical to the replication process of DNA. Without them, the torsional strain in the DNA helix prohibits free rotation, the DNA strands are unable to separate properly, and the cell eventually dies without dividing. Topo I links to the 3′-terminus of a DNA single strand break, while Topo II links to the 5′-terminus of a double strand DNA break. DNA topoisomerase II inhibitors include the following: intercalators such as amsacrine, dactinomycin, daunorubicin, doxorubicin, idarubicin and mitoxantrone; nonintercalators such as etoposide and teniposide; camptothecins including irinotecan (CPT-II) and topotecan. A representative DNA minor groove binder is plicamycin.

The antimetabolites generally exert cytotoxic activity by interfering with the production of nucleic acids by one or the other of two major mechanisms. Some of the drugs inhibit production of the deoxyribonucleoside triphosphates that are the immediate precursors of DNA synthesis, thus inhibiting DNA replication. Some of the compounds are sufficiently like purines or pyrimidines to be able to substitute for them in the anabolic nucleotide pathways. These analogs can then be substituted into the DNA and RNA instead of their normal counterparts. The antimetabolites useful herein include: folate antagonists such as methotrexate and trimetrexate; pyrimidine antagonists such as fluorouracil, fluorodeoxyuridine, azacitidine, cytarabine, and floxuridine; purine antagonists include mercaptopurine, 6-thioguanine, fludarabine, pentostatin; sugar modified analogs include cytarabine, fludarabine; ribonucleotide reductase inhibitors include hydroxyurea.

Tubulin interactive agents interfere with cell division by binding to specific sites on Tubulin, a protein that polymerizes to form cellular microtubules. Microtubules are critical cell structure units. When the interactive agents bind on the protein, the cell cannot properly form microtubules. Tubulin interactive agents include vincristine and vinblastine, both alkaloids and the taxanes (paclitaxel and docetaxel).

Although their mechanisms of action are different, both taxanes and vinca alkaloids exert their biological effects on the cell microtubles. Taxanes act to promote the polymerization of tubulin, a protein subunit of spindle microtubles. The end result is the inhibition of depolymerization of the microtubles, which causes the formation of stable and nonfunctional microtubles. This disrupts the dynamic equilibrium within the microtuble system, and arrests the cell cycle in the late G₂ and M phases, which inhibits cell replication.

Like taxanes, vinca alkaloids also act to affect the microtuble system within the cells. In contrast to taxanes, vinca alkaloids bind to tubulin and inhibit or prevent the polymerization of tubulin subunits into microtubles. Vinca alkaloids also induce the depolymerization of microtubles, which inhibits microtuble assembly and mediates cellular metaphase arrest. Vinca alkaloids also exert effects on nucleic acid and protein synthesis; amino acid, cyclic AMP, and glutathione synthesis; cellular respiration; and exert immunosuppressive activity at higher concentrations.

Antihormonal agents exert cytotoxic activity by blocking hormone action at the end-receptor organ. Several different types of neoplasm require hormonal stimulation to propagate cell reproduction. The antihormonal agents, by blocking hormone action, deprive the neoplastic cells of a necessary stimulus to reproduce. As the cells reach the end of their life cycle, they die normally, without dividing and producing additional malignant cells. Antihormonal agents are typically derived from natural sources and include: estrogens, conjugated estrogens and ethinyl estradiol and diethylstibesterol, chlortrianisen and idenestrol; progestins such as hydroxyprogesterone caproate, medroxyprogesterone, and megestrol; androgens such as testosterone, testosterone propionate; fluoxymesterone, methyltestosterone.

Adrenal corticosteroids are derived from natural adrenal cortisol or hydrocortisone. They are used because of their anti-inflammatory benefits as well as the ability of some to inhibit mitotic divisions and to halt DNA synthesis. These compounds include prednisone, dexamethasone, methylprednisolone, and prednisolone.

Leutinizing-releasing hormone agents or gonadotropin-releasing hormone antagonists are used primarily in the treatment of prostate cancer. These include leuprolide acetate and goserelin acetate. They prevent the biosynthesis of steroids in the testes.

Anti-hormonal agents include antiestrogenic agents such as tamoxifen, antiandrogen agents such as flutamide, and antiadrenal agents such as mitotane and aminoglutethimide.

ODC (or ornithine decarboxylase) inhibitors inhibit cancerous and pre-cancerous cell proliferation by depleting or otherwise interfering with the activity of ODC, the rate limiting enzyme of polyamine biosynthesis important to neoplastic cell growth. In particular, polyamine biosynthesis wherein ornithine is converted to the polyamine, putrescine, with putrescine being subsequently by converted to spermidine and spermine appears to be an essential biochemical event in the proliferation of neoplastic growth in a variety of cancers and cancer cell lines and the inhibition of ODC activity or depletion of ODC in such neoplastic cells has been shown to reduce polyamine levels in such cells leading to cell growth arrest; more differentiated cell morphology and even cellular senescence and death. In this regard, ODC or polyamine synthesis inhibitors are considered to be more cytotoxic agents functioning to prevent cancer reoccurrence or the conversion of pre-cancerous cells to cancerous cells than cytotoxic or cell killing agents. A suitable ODC inhibitor is eflornithine or α-difluoromethyl-ornithine, an orally available, irreversible ODC inhibitor, as well as a variety of polyamine analogs which are in various stages of pre-clinical and clinical research.

Other cytotoxics include agents which interfere or block various cellular processes essential for maintenance of cellular functions or cell mitosis as welt as agents which promote apoptosis. In this regard, hydroxyurea appears to act via inhibitors of the enzyme ribonucleotide reductase whereas asparaginase enzymatically converts asparagine into non-functional aspartic acid thereby blocking protein synthesis in a tumor.

Compositions of the MUC1/ECD antagonists of present invention can also be used in combination with antibodies to HER-2, such as Trastuzumab (Herceptin (H)). In addition, the present invention also encompasses the use of MUC1 domain antagonists in combination with epidermal growth factor receptor-interactive agents such as tyrosine kinase inhibitors. Tyrosine kinase inhibitors suitably include imatinib (Norvartis), OSI-774 (OSI Pharmaceuticals), ZD-1839 (AstraZeneca), SU-101 (Sugen) and CP-701 (Cephalon).

When used in the treatment methods of the present invention, it is contemplated that the chemotherapeutic agent of choice can be conveniently used in any formulation which is currently commercially available, and at dosages which fall below or within the approved label usage for single agent use.

V. Ionizing Radiation

In the present invention, the term “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art. The amount of ionizing radiation needed in a given cell generally depends on the nature of that cell. Means for determining an effective amount of radiation are well known in the art. Used herein, the term “an effective dose” of ionizing radiation means a dose of ionizing radiation that produces cell damage or death when given in conjunction with the MUC1/ECD antagonists of the present invention, optionally further combined with a chemotherapeutic agent.

Dosage ranges for x-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Any suitable means for delivering radiation to a tissue may be employed in the present invention, in addition to external means. For example, radiation may be delivered by first providing a radiolabeled antibody that immunoreacts with an antigen of the tumor, followed by delivering an effective amount of the radiolabeled antibody to the tumor. In addition, radioisotopes may be used to deliver ionizing radiation to a tissue or cell.

VI. Downregulation of MUC1/ECD Expression

The present invention also encompass compounds that downregulate MUC1/ECD expression. One such compound is the isocoumarin NM-3 (2-(8-hydroxy-6-methoxy-1-oxo-1 H-2-benzopyran-3-yl) propionic acid). NM-3 and other isocoumarins suitable to downregulate the expression of MUC1/ECD are disclosed in U.S. Pat. No. 6,020,363, the entirety of which is herein incorporated by reference. Other suitable compounds include 2-substituted estradiol compounds such as 2-methoxyestradiol and 2-hydroxyrestradiol. These and other suitable estradiol derivatives are disclosed in U.S. Pat. No. 6,239,123, the entirety of which is herein incorporated by reference. Other compounds suitable for downregulating MUC1/ECD expression include antisense oligonucleotides that target nucleic acid molecules encoding MUC1, as described below.

VII. Antisense Oligonucleotides and Interfering RNA

The present invention also employs antisense compounds, particularly oligonucleotides, for use in modulating the function of nucteic acid molecules encoding MUC1 and MUC1/ECD wild-type ligands, such as neuregulin 2 isoforms 5 and 6. Inhibition of MUC1 expression will decrease the levels of MUC1/ECD available for binding to MUC1/ECD ligands. Inhibition of the expression of the endogenous ligands of MUC1/ECD will prevent or decrease the proliferative effect on cancer cells associated with the binding of such ligands to MUC1/ECD. Antisense methodology takes advantage of the fact that nucleic acids tend to pair with “complementary sequences.” By complementary, it is meant that polynucteotides are those capable of base-pairing according to the standard Watson-Crick complementary rules. The oligonucleotides of the present invention may be targeted wholly or in part to informational sequences, i.e., those coding for a protein, and other associated ribonucleotides such 5′-untranslated regions, 3′-untranslated regions, 5′ cap regions and intron/exon junctions. Thus, the invention provides oligonucleotides which specifically hybridize with nucleic acids, preferably mRNA, encoding MUC1 and/or MUC1/ECD wild-type ligands such as neuregulin 2 isoforms 5 and 6. The overall effect of interference with mRNA is modulation of expression of neuregulin isoforms 5 and/or 6. Such modulation can be measured in ways that are routine in the art. In addition, effects on cancer cell proliferation or tumor growth can be assessed.

It is understood that an oligonucteotide need not be 100% complementary to its target nucleic acid sequence to be specifically hybridizable. An oligonucleotide is specifically hybridizable when binding of the oligonucteotide to the target interferes with the normal function of the target molecule to cause a Loss of utility, and there is a sufficient degree of complementarity to avoid non-specific binding of the otigonucleotide to non-target sequences under conditions in which specific binding is desired, i.e., under physiological conditions in the case of in vivo assays or therapeutic treatment.

The antisense compounds in accordance with this invention preferably comprise from about 4 to about 50 nucleobases. Particularly preferred are antisense oligonucleotides comprising from about 8 to about 30 linked nucleobases. The oligonucleotides used in accordance with this invention may be conveniently and routinely made through the well-known technique of solid phase synthesis.

The terms “specifically hybridizable” and “complementary” are used to indicate a degree of complementarity sufficient to result in stable and specific binding between the antisense oligonucleotide and the target nucleic acid sequence. An oligonucleotide need not be 100% complementary to its target nucleic acid sequence to be specifically hybridizable. An oligonucleotide considered “specifically hybridizable” when binding of the oligonucleotide to the target interferes with the normal function of the target molecule to cause a loss of utility and decrease in expression of the product protein, and there is a sufficient degree of complementarity to avoid non-specific binding of the oligonucleotide to non-target sequences.

The neuregulin 2 protein family comprises a number of alternatively spliced isoforms (Ring et al., 1999). The coding sequences for neuregulin 2 isoforms 5 and 6 share the same nucleotide sequence from exons 1 through 6 of the neuregulin 2 gene that code for the first 416 amino acids of each protein but differ in the sequence coding for the carboxy terminal 10 amino acids of isoform 5 and for the carboxy terminal 6 amino acids of isoform 6. The coding DNA sequences of exons 1 through 6 are incorporated in SEQ ID NO: 10 through SED ID. NO: 15 respectively. The sequences coding for the first 416 amino acids of isoforms 5 and 6 are nucleotides 313 through 1012 of SEQ ID. NO 10, nucleotides 51-222 of SEQ ID. NO: 11, nucleotides 230-348 of SEQ ID NO: 12, nucleotides 100 through 220 of SEQ ID. NO: 13, nucleotides 111 through 187 of SEQ ID. NO: 14 and nucleotides 123 through 181 of SEQ ID. NO: 15. The sequences coding for the carboxy terminals of isoform 5 and isoform 6 are nucleotides 132 through 164 of SEQ ID NO: 16 and nucleotides 30 through 50 of SEQ ID NO: 17 respectively.

As SEQ ID NO: 16 and SEQ ID NO: 17 are apparently not shared by other neuregulin gene products, in a preferred embodiment, the antisense oligonucleotide comprises a sequence of at least 4 nucleotides that is complementary to a region between nucleotides 132 and 164 of SEQ ID. NO: 16 or nucleotides 30 through 50 of SEQ ID NO: 17. In a more preferred embodiment, the antisense oligonucleotides comprises a sequence of at least 8 nucleotides that is complementary to a region between nucleotides 132 and 164 of SEQ ID. NO: 16 or nucleotides 30 through 50 of SEQ ID NO: 17.

In other embodiments, the antisense oligonucleotide comprises a sequence of at least 4 nucleotides that is complementary to a region between nucleotides 313 through 1012 of SEQ ID. NO 10, or a region between nucleotides 51-222 of SEQ ID. NO: 11, or a region between nucleotides 230-348 of SEQ ID NO: 12, or a region between nucleotides 100 through 220 of SEQ ID. NO: 13, or a region between nucleotides 111 through 187 of SEQ ID. NO: 14, or a region between nucleotides 123 through 181 of SEQ ID. NO: 15. In another embodiment the antisense oligonucleotide is at least 8 nucleotides that is complementary to a region of the one the foregoing nucleotides sequences.

In other embodiments, the antisense oligonucleotide comprises a sequence of at least 4 nucleotides, and preferably a sequence of at least 8 nucleotides, that is complementary to a non-coding region of SEQ ID NOS: 10 through 17.

In other embodiments of the invention, MUC1 directed antisense oligonucleotides comprise a sequence of at least 4 nucleotides that is complementary to SEQ ID NO: 18. In preferred embodiments, the antisense oligonucleotides comprises a sequence of at least 8 nucleotides that is complementary to SEQ ID NO: 18.

The present invention also encompasses expression vectors comprising an expression control system that directs production of a transcript of the foregoing antisense oligonucleotides. In addition, the present invention provides for methods of hybridization comprising providing one of the forgoing antisense oligonucleotides and contacting such oligonucleotide with a nucleic acid comprising the target sequence under conditions that permit hybridization of the oligonucteotide with the nucleic acid. Also included are methods of inhibiting translation of mRNA comprising providing one of the forgoing antisense oligonucleotides and providing a cell comprising mRNA comprising the target sequence and introducing the oligonucteotide into the cell, wherein the oligonucteotide inhibits translation of the mRNA in the cell.

The present invention also encompasses the use of RNA interference (“RNAi”) molecules, including small interfering RNA (“siRNA”) molecules, as a method of MUC1 gene silencing. siRNA's for mammalian systems are typically composed of double stranded RNA with 19 to 28, preferable 19 to 23, nucleotide RNA strands, a 2 nucleotide overhand at the 3′ end and an optional 5′ phosphate group (Yang et al., 2001; Elbashir et al., 2002). Such siRNA's provide a highly active and selective method for reducing the expression of targeted genes by utilizing the RNA interference post-translational gene silencing pathway. Interference of gene expression by interfering RNA is recognized as a naturally occurring mechanism for silencing alleles during development in plants, invertebrates and vertebrates. In this pathway, it is believed that siRNA form a protein complex, sometimes termed an “RNA-induced silencing complexes” (“RISC”), that serve to guide a nucleoside to the mRNA whose sequence matches that of the siRNA, resulting in cleavage of that mRNA (Zamore, 2001). Studies on a variety of gene products of different functions and subcellular localizations have demonstrated the general applicability of the siRNA technique of gene silencing (Harborth et al., 2001).

In some embodiments, double stranded siRNA complexes are designed using the following guidelines:
(1) a double stranded RNA complex is composed of a 21-nucleotide sense and 21-nucleotide anti-sense strand, both with a 2-nucleotide 3′ overhang, i.e., a 19 nucleotide complementary region;
(2) a 23 nucleotide sequence is chosen in the coding region of the mRNA with a G:C ratio as close to 50% as possible, preferably within about 60% to about 40%, or alternatively within about 70% to about 30% (to create a 21 base pair duplex with overhangs that match the target sequence and have a 19 base pair complementary region, a target sequence of 23 base pairs is needed);
(3) preferably avoid regions within about 75 nucleotides of the AUG start codon or within about 75 nucleotides of the termination codon;
(4) preferably avoid more than three guanosines in a row as poly G sequences can hyperstack and agglomerate;
(5) preferably choose a sequence that starts with AA as this results in siRNA's with dTdT overhangs that are potentially more resistant to nucleases;
(6) preferably the sequence is not homologous to other genes to prevent silencing of unwanted genes with a similar sequence.
   A negative control may be included, such a negative control being a nucleotide sequence from a database for a non-existing gene.

Examples of such 21 nucleotide target DNA sequences, and the 19 nucleotide sense and antisense sequences utilizing dTdT 3′ overhangs (dT is 2′-deoxythymidine), derived from the sequence of MUC1 mRNA (SEQ ID NO: 19), and preferably the coding sequence of MUC1 mRNA (SEQ ID NO: 20), include, but are not limited to, those described in TABLE 1.

TABLE 1

| | | |
|---|---|---|
| Target DNA | sense RNA | antisense RNA |

| | | |
|---|---|---|
| aaaaggagacttcggctaccc | aaggagacuucggcugcccdtdt | gggcagccgaagucuccuudtdt |
| SEQ ID NO: 21 | SEQ ID NO: 22 | SEQ ID NO: 23 |
| aaaggagacttcggctaccca | aggagacuucggcuacccadtdt | uggguagccgaagucuccudtdt |
| SEQ ID NO: 24 | SEQ ID NO: 25 | SEQ ID NO: 26 |
| aaggagacttcggctacccag | ggagacuucggcuacccagdtdt | cuggguagccgaagucuccdtdt |
| SEQ ID NO: 27 | SEQ ID NO: 28 | SEQ ID NO: 29 |
| aaccagcttcaggttcagctg | ccagcuucagguucagcugdtdt | cagcugaaccugaagcuggdtdt |
| SEQ ID NO: 30 | SEQ ID NO: 31 | SEQ ID NO: 32 |
| aacggcacctctgccagggct | cggcaccucugccagggcudtdt | agcccuggcagaggugccgdtdt |
| SEQ ID NO: 33 | SEQ ID NO: 34 | SEQ ID NO: 35 |
| aagactgatgccagtagcact | gacugaugccaguagcacudtdt | agugcuacuggcaucagucdtdt |
| SEQ ID NO: 36 | SEQ ID NO: 37 | SEQ ID NO: 38 |
| aattguctctggccttccgag | uugacucuggccuuccgagdtdt | cucggaaggccagagucadtdt |
| SEQ ID NO: 39 | SEQ ID NO: 40 | SEQ ID NO: 41 |
| aaggtaccatcaatgtccacg | gguaccaucaauguccacgdtdt | cguggacauugaugguaccdtdt |
| SEQ ID NO: 42 | SEQ ID NO: 43 | SEQ ID NO: 44 |
| aatgtccacgacgtgaagaca | uguccacgacgugaagacadtdt | ugucuucacgucguggacadtdt |
| SEQ ID NO: 45 | SEQ ID NO: 46 | SEQ ID NO: 47 |
| aatcagtataaaacggaggca | ucaguauaaaacggaggcadtdt | ugccuccguuuuauacugadtdt |
| SEQ ID NO: 48 | SEQ ID NO: 49 | SEQ ID NO: 50 |
| aaaacggaagcagcctctcga | aacggaagcagccucucggdtdt | ucgacaggcugcuuccguudtdt |
| SEQ ID NO: 51 | SEQ ID NO: 52 | SEQ ID NO: 53 |
| aaacggaagcagcctctcgat | acggaagcagccucucgaudtdt | aucgagaggcugcuuccgudtdt |
| SEQ ID NO: 54 | SEQ ID NO: 55 | SEQ ID NO: 56 |
| aacggaagcagcctctcgata | cggaagcagccucucgauadtdt | uaucgagaggcugcuuccgdtdt |
| SEQ ID NO: 57 | SEQ ID NO: 58 | SEQ ID NO: 59 |
| aagaactacgggcagctggac | gaccuacgggcagcuggacdtdt | auccagcugcccguaguucdtdt |
| SEQ ID NO: 60 | SEQ ID NO: 61 | SEQ ID NO: 62 |

The orientation of the double stranded RNA complex for SEQ ID NO: 22 and SEQ ID NO: 22 is as follows:

```
        5′-aaggagacuucggcugcccdtdt-3′
              |||||||||||||||||||
3′-dtdtuuccucugaagccgacggg-5′
```

The above guidelines are solely an aid to designing suitable RNA oligonucleotides and is not a imitation of the interfering RNA oligonucleotides and related methods of use of the present invention. Thus, also included in the invention are target sequences such as aagggggttttctgggcctct (SEQ ID NO: 63) and the sense siRNA sequence ggggguuuucugggccucudtdt (SEQ ID NO: 64) and the siRNA antisense sequence agaggcccagaaaacccccdtdt (SEQ ID NO: 65); target sequence aagttcagtgcccagctctac (SEQ ID NO: 66) and the sense siRNA sequence guucagugcccagcucuacdtdt (SEQ ID NO: 67) and the antisense siRNA sequence guagagcugggcacugaacdtdt (SEQ ID NO: 68); and target sequence aaggtttctgcaggtaacggt (SEQ ID NO: 69) and the sense siRNA sequence gguuucugcagguaauggudtdt (SEQ ID NO: 70) and the antisense siRNA sequence accauuaccugcagaaaccdtdt (SEQ ID NO: 71). Control siRNA sequences include those derived from scrambled target sequences such as gcgcgctttguaggattcg (SEQ ID NO: 72) and the sense siRNA sequence gcgcgcuuuguaggauucgdtdt (SEQ ID NO: 73) and the antisense siRNA sequence cgaauccuacaaagcgcgcdtdt (SEQ ID NO: 74).

Also encompassed by the present invention are double stranded RNA complexes wherein the antisense strand is not exactly complementary to the target mRNA sequence, but can stilt downregulate MUC-1 expression. Thus, in some embodiments, the antisense strand is a sequence that will hybridize under stringent conditions to the target mRNA sequence. Stringent conditions as used herein means hybridization to filter-bound DNA in 0.5 M NaHPO₄, 7% sodium dodecyl sulfate (SDS), 1 mM EDTA at 65° C., and washing in 0.1× sodium chloride/sodium citrate (SSC)/0.1% SDS at 68° C. (Ausubel et at., eds., 1989, Current Protocols in Molecular Biology, Vol. I, John Wiley & Sons, Inc., New York, at p. 2.10.3). In other embodiments, the antisense strand is a sequence that is substantially complementary to the target mRNA sequence. Substantially complementary means that the sequence has up to four mismatched base pairs with the caveat that the double stranded RNA complex can still effect the downregulation of MUC1. Downregulation of MUC1 is determined by inhibition in protein expression by Western blot analysis using specific anti-MUC1 antibodies and/or a RT-PCR analysis specific for MUC1 RNA as compared to a suitable control. In other embodiments, the sense strand has at least a 60% sequence identity to the target mRNA sequence, with the caveat that that the double stranded RNA complex can still effect the downregulation of MUC1. The extent of sequence identity may be greater than 60%, such as at least 65%, at least 70%, at least 75%, at least 80%, at least 85%, or at Least 90% sequence identity. “Sequence identity” as used herein, refers to the subunit sequence similarity of two polymeric molecules, herein oLigonucLeotides. The identity between two sequences is a direct function of the numbering of matching or identical positions. Identity can be measured using the sequence analysis software BLASTN. The default parameters for comparing two sequences by BLASTN are reward for match=1, penalty for mismatch=−2, open gap=5, extension gap=2.

The double-stranded siRNA complexes of the present invention also encompass hair-pin RNA, in which both strands of a siRNA duplex is included within a single RNA oligonucleotide (Yu et at., 2002; Devroe et al., 2002; Brummelkamp et at., 2002). Thus, for example, the forgoing exemplified complementary sense and antisense RNA sequences may be incorporated into single hairpin RNA oligonucleotides.

In addition to the use of double stranded siRNA complexes, single strand antisense RNA oligonucleotides can also result in gene silencing utilizing the interference pathway (Martinez et al., 2002). Such single strand antisense RNA is preferably 5′ phosphorylated and in mammalian systems is effective from 17 to at least 29 nucleotides in length (Martinez et al., 2002) and in C. elegans from between 22 and 40 nucleotides in length (Tijsterman et al., 2002). Thus, one aspect of the present invention is a 5′ phosphorylated RNA oligonucleotide of 17 to 40 bases that will hybridize under stringent conditions to SEQ ID NO: 19, wherein SED ID NO: 19 represents MUC1 mRNA, or preferably, that will hybridize under stringent conditions to SEQ ID NO: 20, wherein SEQ ID NO: 20 represents the sequence that codes for MUC1. Stringent conditions for hybridization are as defined above. Another aspect of the present invention are 5′ phosphorylated RNA oligonucleotides of 17 to 40 bases, wherein the sequences are substantially complementary to a sequence of an equivalent number of bases found in SEQ ID NO: 19, and preferably in SEQ ID NO: 20, and wherein the oligonucleotide will downregulate MUC1 expression in a MUC1 expressing cell. Substantially complementary means that the antisense sequence of the double stranded siRNA complex has up to four mismatched base pairs as compared with the target mRNA sequence, with the caveat that the 5′ phosphorylated RNA oligonucleotide of 17 to 40 bases can still effect the downregulation of MUC1. Another aspect of the invention are 5′ phosphorylated RNA oligonucleotide of 17 to 40 bases, wherein the sequences have at least a 60% sequence identity to a sequence of an equivalent number of bases in SEQ ID NO: 75, the antisense sequence complementary to the coding region of MUC1 mRNA, and wherein the oligonucleotide will downregulate MUC1 expression in a MUC1 expressing cell. Examples of such sequences include, but are not limited to, the 5′ phosphorylated derivative of the following 40 nucleotide antisense RNA oligonucleotide sequences, plus 5′ phosphorylated sequences of 19, 20, 21, 22, 23, 24, 25, 26, 27, 28, 29, 30, 31, 32, 33, 34, 35, 36 3,7 38, and 39 nucleotides in length formed by the removal of contiguous nucleotides from the 3′ terminus of the following oligonucleotide sequences:

| | |
|---|---|
| 5′-cucauaggggcuacgaucgguacugcuag | (SEQ ID NO: 76) |
| ggggcacauag-3′ | |
| 5′-cagcugcccguaguucuuucggcggcacu | (SEQ ID NO: 77) |
| gacagacagcc-3′ | |
| 5′-agacaaugccagcgcaaccagaacacaga | (SEQ ID NO: 78) |
| ccagcaccagc-3′ | |
| 5′-gccuggcaccccagccccagacugggcag | (SEQ ID NO: 79) |
| agaaaggaaau-3′ | |
| 5′-gcugacgucugagaucgucagguuauauc | (SEQ ID NO: 80) |
| gagaggcugcu-3′ | |
| 5′-auugaacugugucuccacgucguggacau | (SEQ ID NO: 81) |
| ugaugguaccu-3′ | |
| 5′-agucaauuguaccaccacagauccuggcc | (SEQ ID NO: 82) |
| ugaacuuaaua-3′ | |
| 5′-aaaacccccuuguuuauaaaucugcaaaa | (SEQ ID NO: 83) |
| acauuucagaa-3′ | |
| 5′-cucuugguaguagucggugcugggaucuu | (SEQ ID NO: 84) |
| ccagagaggaa-3′ | |
| 5′-ugaaaugugaaaagacaggaaaaagaaag | (SEQ ID NO: 85) |
| agaccccagua-3′ | |
| 5′-agugcugugauuggaggaggugagaggag | (SEQ ID NO: 86) |
| guaccgugcua-3′ | |
| 5′-ggcaucagucuuggugcuauggcuggcaa | (SEQ ID NO: 87) |
| gggugguagga-3′ | |
| 5′-agaggugccguugugcaccagaguagaag | (SEQ ID NO: 88) |
| cugagccugau-3′ | |
| 5′-cagggcuggccuggugacugggaccgagg | (SEQ ID NO: 89) |
| ugacauccugu-3′ | |
| 5′-cacagcauucuucucaguagagcugggca | (SEQ ID NO: 90) |
| cugaacuucuc-3′ | |
| 5′-cgcucauaggaugguagguaucccgggcu | (SEQ ID NO: 91) |
| guaaagauguc-3′ | |
| 5′-ucuuucggcggcacugacagacagccaag | (SEQ ID NO: 92) |
| gcaaaugagau-3′ | |
| 5′-caaccagaacacagaccagcaccagcagc | (SEQ ID NO: 93) |
| gcgaugcccca-3′ | |
| 5′-ccccagacugggcagagaaaggaaauggc | (SEQ ID NO: 94) |
| acaucacucac-3′ | |
| 5′-ucgucagguuauaucgagaggcugcuucc | (SEQ ID NO: 95) |
| guuuuauacug-3′ | |
| 5′-ccacgucguggacauugaugguaccuucu | (SEQ ID NO: 96) |
| cggaaggccag-3′ | |
| 5′-ccacagauccuggccugaacuuaauauug | (SEQ ID NO: 97) |
| gagaggcccag-3′ | |
| 5′-uauaaaucugcaaaaacauuucagaaaug | (SEQ ID NO: 98) |
| ucucucugcag-3′ | |
| 5′-cggugcugggaucuuccagagaggaauua | (SEQ ID NO: 99) |
| aacuggagguu-3′ | |
| 5′-aggaggugagaggagguaccgugcuaugg | (SEQ ID NO: 100) |
| ugagugcuacu-3′ | |
| 5′-ugcuauggcuggcaagggugguaggagua | (SEQ ID NO: 101) |
| ucagaguggug-3′ | |
| 5′-gcaccagaguagaagcugagccugaugca | (SEQ ID NO: 102) |
| gagccugaggc-3′ | |
| 5′-ugacugggaccgaggugacauccuguccc | (SEQ ID NO: 103) |
| agguggcagcu-3′ | |
| 5′-caguagagcugggcacugaacuucucugg | (SEQ ID NO: 104) |
| guagccgaagu-3′ | |

Another aspect of the present invention include methods to inhibit MUC1 expression, MUC1-mediated signaling events that leads to inhibition of tumor cell proliferation and induction of tumor cell apoptosis comprising delivering a single stranded antisense RNA of the present invention into a cell that expresses MUC1.

siRNA oligonucleotides can be synthesized, annealed when required, and purified by methods known in the art (see e.g., Elbashir et al., 2002, herein incorporated by reference). Cells may be transfected with siRNA by use of liposomal and other lipid-mediated transfection methodologies (Hohjoh, 2002; Bertrand et al., 2002; Elbashir et al., 2002, all herein incorporated by reference). Alternatively, siRNA's may be expressed in cells transfected with suitable expression cassettes or vectors (Brummelkamp et al., 2002; Sui et al., 2002; Paul et al., 2002) and by the use of viral mediated delivery mechanisms, e.g., adenoviral and retroviral systems, that may be suitably used to express siRNA in vitro and in vivo (Xia et al., 2002; Devroe & Silver, 2002). In addition to delivery of siRNA molecules, the present invention also encompasses the delivery of longer RNAi molecules by expression constructs. These longer RNAi molecules may effect gene silencing directly or subsequent to enzymatic cleavage by Dicer. The longer RNAi molecule may be a dsRNA molecule wherein the sense is SEQ ID NO: 19 or SEQ ID NO: 20 or a fragment thereof, or in one embodiment is a dsRNA molecule of substantially equivalent size of a dsRNA molecule wherein the sense is SEQ ID NO: 19 or SEQ ID NO: 20, wherein substantially similar means±10% relative to the number of bp in the aforementioned dsRNA molecules wherein the sense is SEQ ID NO: 19 or SEQ ID NO: 20, wherein the antisense strand will hybridize with SEQ ID 19 or SEQ ID 20 under stringent conditions, as defined previously, or in another embodiment are substantially complementary, as defined previously, to SEQ ID 19 or SEQ ID 20, or in another embodiment the sense strand has at least 60% sequence identity, as previously defined, to SEQ ID NO 19 or SEQ ID NO: 20. In various embodiments, The extent of sequence identity may be greater than 60%, such as at least 65%, at least 70%, at least 75%, at least 80%, at least 85%, or at least 90% sequence identity. In other embodiments, the longer the antisense strand of a dsRNAi molecule may comprise one or more of the sequences SEQ ID NO: 76 though SEQ ID NO: 104, wherein the dsRNAi molecule is about 100 bp, or about 150 bp, or about 200 bp, or about 250 bp, or about 300 bp, or about 350 bp, or about 400 bp in length.

In the context of the present invention, the term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases.

In some embodiments, the oligonucleotides of the present invention may comprise one or more modified internucleoside linkage. Modifications of the normal 3′ to 5′ phosphodiester linkage include phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′linkages, 2′-5′ linked analogs of these, and those having inverted polarity wherein one or more internucleotide linkages is a 3′ to 3′, 5′ to 5′ or 2′ to 2′ linkage. Examples of foregoing are taught in WO9905160 and U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697, 5,625,050, 5,652,355, 5,652,356 and 5,750,674, all of which are herein incorporated by reference.

Other non-phosphorus containing modified linkages include those formed by short chain alkyl or cycloalkyl internucleoside linkages, mixed heteroatom and alkyl or cycloalkyl internucleoside linkages, or one or more short chain heteroatomic or heterocyclic internucleoside linkages. Examples include morpholino, siloxane, sulfide, sulfoxide, sulfone, sulfonate, sulfonamide, formacetyl, thioformacetyl, riboacetyl, atkene, sulfamate, methyleneimino, methylenehydrazino, amide backbones; and others having mixed N, O, S, and methylene parts. Examples of foregoing are taught in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, all of which are herein incorporated by reference.

In other embodiments, the oligonucleotides of the present invention may comprise one or more modified sugars, including substituted sugars and sugar mimetics. Examples of 2′ substitutents include OH, halo, amino, cyano, or O, S or N linked alkyl, alkenyl or alkynyl groups, wherein the alkyl, alkenyl and alkynyl groups may be substituted or unsubstituted C₁-C₁₀ alkyl or C₂-C₁₀ alkenyl and alkynyl, or, alkoxyalkoxy, heterocycloalkyl, heterocycloalkaryl, aminoalkylamino, polyalkylamino, or substituted silyl. Examples include 2′-dimethylaminooxyethoxy, 2′-dimethylaminoethoxyethoxy, 2′-methoxy, 2′-aminopropoxy, 2′-CH₂—CH═CH₂, 2′-O—CH₂—CH═CH₂, and 2′-fluoro. The 2′-modification may be in the arabino position or ribo position. Substitutions at the 2′ site of sugars also include Locked Nucleic Acids (LNAs) wherein the 2′-hydroxyl group is linked to the 3′ or 4′ carbon atom of the sugar ring thereby forming a bicyclic sugar moiety. In one embodiment a —CH₂— or —CH₂CH₂— group bridges the 2′ oxygen atom and the 4′ carbon atom. Similar modifications may also be made at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Examples of foregoing are taught in U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920, and 6,268,490 and U.S. application No. 20020068708A1, all of which are herein incorporated by reference.

In some embodiments, both the sugar and the internucleoside linkages are modified or replaced with novel groups. One such example is referred to as a peptide nucleic acid (PNA) wherein the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Examples of foregoing are taught in U.S. Pat. Nos. 5,539,082; 5,714,331; 5,719,262, and 6,395,474, all of which are herein incorporated by reference.

In further embodiments, the oligonucleotides of the present invention may comprise one or more modified nucleobase. As used in the context of the oligonucleotides of the present invention, “unmodified” nucleobases include the purine bases adenine and guanine, and the pyrimidine bases thymine, cytosine and uracil. Modified nucleobases include other synthetic and natural occurring nucleobases such as 2,6-diamonopurine, 5-methylcytosine, 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-fluoro-adenine, 2-amino-adenine, 8-azaguanine and 8-azaadenine, 7-deazaguanine and 7-deazaadenine and 3-deazaguanine and 3-deazaadenine. Other examples include tricyclic pyrimidines such as phenoxazine cytidine, phenothiazine cytidine, phenoxazine cytidine, carbazole cytidine, and pyridoindole cytidine. Modified nucleobases may also include those in which the purine or pyrimidine base is replaced with other heterocycles, for example 7-deaza-adenine, 7-deazaguanosine, 2-aminopyridine and 2-pyridone. Examples of foregoing are taught in U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 5,681,941; 5,750,692, 6,005,096; 6,414,112 and Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993, all of which are herein incorporated by reference.

In still further embodiments, the oligonucleotides of the present invention may be linked to one or more moieties or conjugates which enhance the activity, tissue distribution, and/or cellular uptake of the oligonucleotides. Such moieties include but are not limited to, N-9-2-hydroxypropyl)methacrylamide copolymer (Jensen et al., 2002) cholesterol (Letsinger, 1989), cholic acid (Manoharan et al., 1994), a thioether, (Manoharan et al., 1992; Manoharan et al., 1993), a thiocholesterol (Oberhauser et al., 1992), an aliphatic chain, such as dodecandiol or undecyl residues (Saison-Behmoaras et al., 1991; Kabanov et al., 1990; Svinarchuk et al., 1993), a phospholipid, e.g., di-hexadecyl-rac-glycerol or triethyl-ammonium 1,2-di-O-hexadecyl-rac-glycero-3-H-phosphonate (Manoharan et al., 1995; Shea et al., 1990), a polyamine or a polyethylene glycol chain (Manoharan et al., 1995), or adamantane acetic acid (Manoharan et al., 1995), a palmityl moiety (Mishra et al., 1995), or an octadecylamine or hexylamino-carbonyl-oxycholesterol moiety (Crooke et al., 1996) or peptides including delivery peptides, e.g., Antennapaedia peptide (Fischer et al., 2002; Zatsepin et al., 2002; Oehlke et al., 2002). Further examples that teach the preparation of such oligonucleotide conjugates include U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, all of which are herein incorporated by reference.

Another aspect of the present invention provides for pharmaceutical compositions comprising an oligonucleotide of the present invention and a pharmaceutically acceptable carrier.

VIII. Vaccines

The present invention also encompasses the use of MUC1/ECD peptides, e.g., SEQ ID NO: 1 or fragments thereof, wherein such fragments comprise four or more consecutive amino acids of SEQ ID NO. 1, in a vaccine wherein the host mammal generates antibodies to the polypeptide which also act against the host's own MUC1/ECD. Vaccine preparation techniques are generally known in the art as described by Duffy (1980), and references cited therein, all of which are incorporated herein by reference.

The MUC1/ECD peptides may be conjugated to a carrier molecule such as a protein or Ficoll. The carrier protein is preferably one with a molecular weight of at least about 40,000 dalton and more preferably at least about 60,000 dalton. The vaccine formulation may comprise a pharmaceutically acceptable carrier and may also include adjuvant systems for enhancing the immunogenicity of the formulation, such as oil-in water systems and other systems known in the art. Since the peptides or conjugates may be broken down in the stomach, the vaccine is preferably administered parenterally (for instance, subcutaneous, intramuscular, intravenous, or intradermal injection). The dosage will depend on the specific activity of the vaccine and can be readily determined by routine experimentation. The formulations may be presented in unit-dose or multi-dose containers, for example, sealed ampoules and vials and may be stored in a freeze-dried condition requiring only the addition of the sterile liquid carrier immediately prior to use.

IX. Formulations

The MUC1/ECD antagonists including binding inhibitors or oligonucleotides employed in the compositions and methods of the present invention can be formulated in a variety of conventional pharmaceutical formulations and administered to cancer patients, in need of treatment, by any one of the drug administration routes conventionally employed including oral, intravenous, intraarterial, parental or intraperitoneal.

For oral administration the compositions of the present invention may be formulated, for example, with an inert dilutent or with an assimiable edible carrier, or enclosed in hard or soft shell gelatin capsules, or compressed into tablets, or incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations may, of course, be varied and may conveniently be between about 2 to about 60% of the weight of the unit. The amount of active compounds in such therapeutically useful compositions is such that a suitable dosage will be obtained.

The tablets, troches, pills, capsules and the like may also contain the following: a binder, a gum tragacanth, acacia, cornstarch, or gelatin; excipients, such as dicalcium phosphate; a disintegrating agent, such as corn starch, potato starch, alginic acid and the Like; a Lubricant, such as magnesium stearate; and a sweetening agent, such as sucrose, Lactose or saccharin may be added or a flavoring agent, such as peppermint, oil of wintergreen, or cherry flavoring. When the dosage unit for is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compounds sucrose as a sweetening agent methyl and propylparabens as preservatives, a dye and flavoring, such as cherry or orange flavor. Of course, any material used in preparing a dosage unit form should be pharmaceutically pure and substantially non-toxic in the amounts employed. In addition, other chemotherapeutic compounds may be incorporated into sustained-release preparation and formulations.

In regard to formulations comprising oligonucleotides, colloidal dispersion systems may be used as delivery vehicles to enhance the in vivo stability of the oligonucleotides and/or to target the oligonucleotides to a particular organ, tissue or cell type. Colloidal dispersion systems include, but are not limited to, macromolecule complexes, nanocapsules, microspheres, beads and lipid-based systems including oil-in-water emulsions, micelles, mixed micelles, liposomes and lipid: oligonucleotide complexes of uncharacterized structure.

Pharmaceutical formulations of the compositions of the present invention which are suitable for injectable use include sterile aqueous solutions or dispersions and sterile powders for the extemporaneous preparation of sterile injectable solutions or dispersions. In all cases the form must be sterile and must be fluid to the extent that each syringability exists. It must be stable under the conditions of manufacture and storage and must be preserved against the contaminating action of microorganisms, such as bacteria and fungi. The carrier can be a solvent or dispersion medium containing, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the compositions of the present invention in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the composition.

X. Treatment Methods

Tumors that can be suitably treated with the methods of the present invention include; but are not limited to, tumors of the brain (glioblastomas, medulloblastoma, astrocytoma, oligodendroglioma, ependymomas), lung, liver, spleen, kidney, lymph node, small intestine, pancreas, blood cells, colon, stomach, breast, endometrium, prostate, testicle, ovary, skin, head and neck, esophagus, bone marrow, blood and other tissue. The tumor may be distinguished as metastatic and non-metastatic. Pre-malignant lesions may also be suitably treated with the methods of the present invention.

The treatment with the MUC1/ECD antagonists of the present invention may precede or follow irradiation and/or chemotherapy by intervals ranging from seconds to weeks and/or be administered concurrently with such treatments. In embodiments where the MUC1/ECD antagonists and irradiation and/or chemotherapy are applied separately to the cell, steps should be taken to ensure that a significant period of time does not expire between the time of each delivery, such that the combination of the two or three treatments would still be able to exert an advantageously combined effect on the cell. In such instances, it is contemplated that one would contact the cell with the treatment agents or modalities within amount 0.1 to 25 h of each other and, more preferably, within about 1 to 4 h of each other, with a delay time of only about 1 h to about 2 h being most preferred. In some situations, it is desirable to extend the time period of treatment significantly, however, where several days (2, 3, 4, 5, 6 or 7) or several weeks (1, 2, 3, 4, 5, 6, 7 or 8) lapse between the respective administrations. In any case, the invention contemplates that the MUC1/ECD antagonists may be given before, after or even simultaneously with the ionizing radiation and/or chemotherapeutic agent.

Some chemotherapeutic agents transiently induce MUC1 expression on cancer cells 0.5 to 12 hours after contact of the carcinoma cells with the chemotherapeutic agents. Thus, in some embodiments the administration of MUC1/ECD binding inhibitors, especially antibodies to the MUC/ECD sequence SEQ ID NO. 1, optionally conjugated to a toxin or radionucleotide, is coordinated with the increased expression of MUC1 on the cancer cells. In other embodiments, agents other than chemotherapeutic agents may be used to increase MUC1 expression prior to treatment with a MUC1 binding inhibitor, especially antibodies to the MUC/ECD sequence SEQ ID NO. 1, optionally conjugated to a toxin or radionucleotide.

In the methods of the present invention, the actual dosage of MUC1/ECD antagonists employed will depend on a variety of factors including the type and severity of cancer being treated, and the additive or synergistic treatment effects of the MUC1/ECD antagonists and the other treatment modality or modalities selected.

### EXAMPLES OF THE INVENTION

### Example 1

### Peptides

The MUC1/ECD polypeptide sequence, as typically found in MUC1-expressing cells, is provided by SEQ ID NO: 1. A number of polypeptide sequences have been synthesized by standard techniques. These include peptides P1 (SEQ ID NO: 2), P2 (SEQ ID NO: 5) and P3 (SEQ ID NO:6), which are polypeptide fragments of MUC1/ECD. PI (SEQ ID NO: 2) represents amino acids 5 through 20 of MUC1/ECD (SEQ ID NO: 1) with a cysteine added at the carboxy terminal. P2 (SEQ ID NO: 5) represents amino acids 13 through 28 of MUC1/ECD (SEQ ID NO: 1) with a cysteine added at the carboxy terminal. P3 (SEQ ID NO: 6) represents amino acids 27 through 44 of MUC1/ECD (SEQ ID NO: 1) with a cysteine at the carboxy terminal. In addition, the synthesized polypeptide sequence SEQ ID NO: 7 incorporates amino acids 6 through 24 of MUC1/ECD (SEQ ID NO: 1) with a cysteine added at the carboxy terminal. The synthesized polypeptide P0 (SEQ ID NO: 8) incorporates a 19 amino acid sequence occurring in the MUC1 protein occurring just prior to the amino terminus of MUC1/ECD and represents the a potential cleavage site. A cysteine was again added at the carboxy terminal of the sequence at it occurs in the MUC 1 sequence.

### Example 2

### Anti-MUC1-P1 Antibody

A. Generation of Antibody

The polypeptide P1 (SEQ. ID NO: 4), contains the QYK motif and other sequences homologous to ligand binding domains of cytokine receptors (Zrihan-Licht, et al., 1994). A polyclonal antibody was raised in rabbits against polypeptide P1 (SEQ ID NO. 4) conjugated to KLH. Serum was prepared by standard methods.

Polyclonal antibodies were also raised against the polypeptide SEQ ID NO: 7, whereby the immunogen was formed by conjugating the potypeptide SEQ ID NO: 7 to KLH. Antibodies have been obtained from 2 rabbits, designated 3402-1 and 3402-2. Both serum and affinity purified antibody preparations have been prepared by standard methodologies.

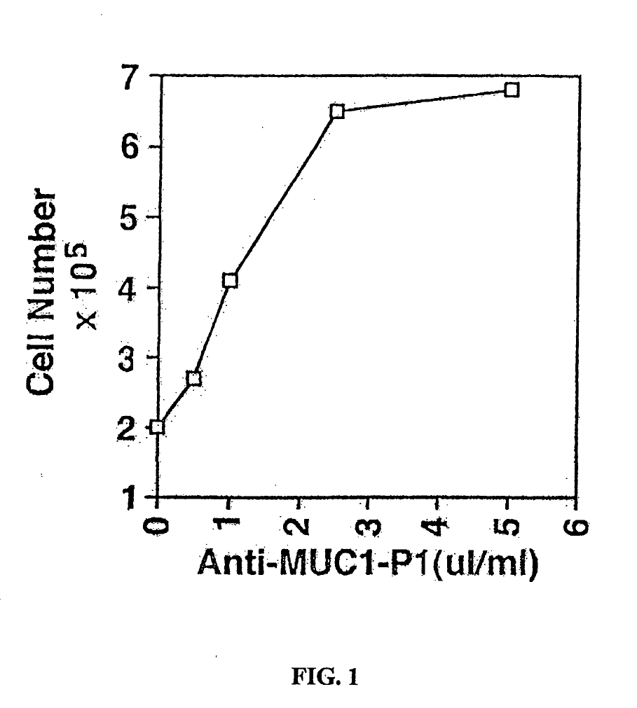

B. Stimulation of Human Carcinoma Cells by Anti-MUC1-P1-Antibody Human ZR-75-1 carcinoma cells were grown to 80% confluence in RPMI 1640 medium containing 10% fetal bovine serum (FBS) and then passed onto a 6-well plate at 1×10⁴ cells per well. After overnight starvation in medium containing 0.1% FBS, anti-MUC1-P1 antibody was added to each well it the amounts indicated in FIG. 1 and incubated for 48 hours. Cell numbers were quantified after another 3 days of incubation in the presence of 0.1% FBS. As shown in FIG. 1, anti-MUCi-P1 stimulates the growth of ZR-75-1 cells in a dose dependent fashion.

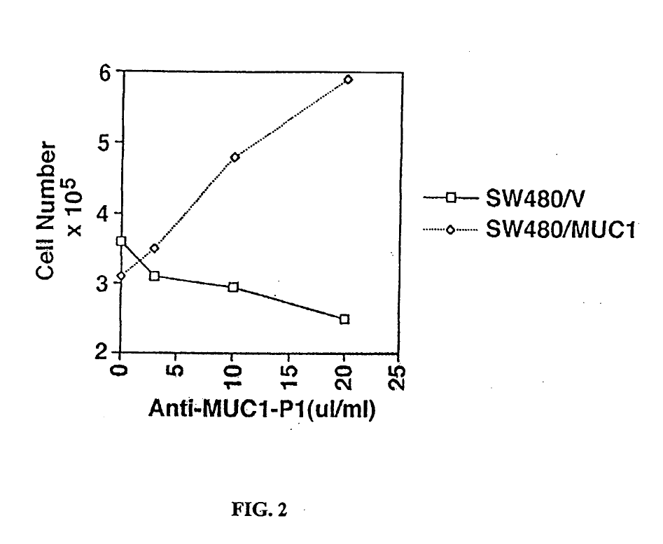

To assess the specificity of anti-MUC1-P1 antibody stimulation, human MUC1-negative SW480 colon cancer cells were stably transfected to express empty vector (SW480/V) or MUC1 (SW480/MUC1). SW80 colon cancer cells were transfected with either pCMV-IE-ak1-dhfr vector or pCMV-IE-ak1-dhfr-MUC1 using lipofectamide (Ligtenburger et al., 1992). Cells were grown in the presence of 800 μg/ml G418 (neomycin) and serially diluted to single-cell populations. Single cell clones that express MUC1 (SW480/MUC1) were selected. Both SW480 cells types were grown to 80% confluence in DMEM containing 10% FBS and plated onto 6-well plates at 5×10⁴ cells per well. After overnight starvation in medium containing 0.1% FBS, anti-MUC1-P1 antibody was added at the indicated concentrations and incubated for 48 hr. Cell numbers were quantified after a further day of incubation (3 days total). As shown in FIG. 2, anti-MUC1-P1 stimulates the growth of SW480/MUC1 cells but not SW48-0/V cells. These findings confirm that the anti-MUC1-P1 antibody stimulates the growth of human carcinoma cells by specifically interacting with MUC1.

### Example 3

### Endogenous MUC1 ECD Ligands

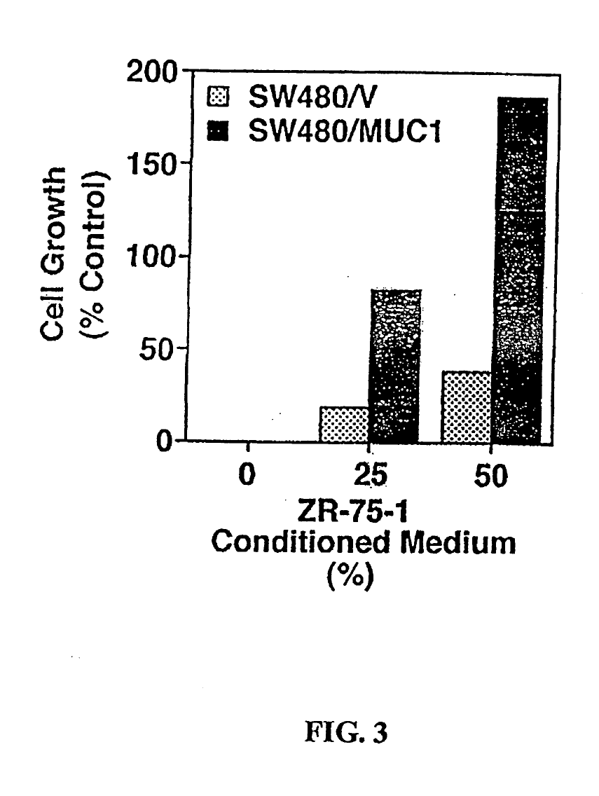

The finding that anti-MUC1-P1 stimulates growth of human carcinoma cells is suggestive of the potential existence of natural MUC1 ECD ligands. To investigate this potential, ZR-75-1 cells were screened as a possible source of MUC1 ligand(s). Conditioned medium was prepared by culturing ZR-75-1 cells in RPMI 1640 medium containing 0.1% FBS for 72 hr and then preparing a supernatant. The conditioned medium was added to SW480/V and SW480/MUC1 cells that were growth arrested in DME containing 0.1% FBS as previously described. Conditioned medium was added at the concentration indicated in FIG. 3 and the cells were maintained for 3 days prior to quantification of cell numbers. As shown in FIG. 3, ZR-75-1 cells express a soluble ligand that stimulates carcinoma cell growth by binding to MUC1.

To identify the soluble MUC1 ligand(s), a fusion protein comprising the MUC1/ECD (SEQ ID. No: 1) and glutathione S-transferase (GST) was prepared. GST was amplified by PCR using a set of primers as follows:
5′-ATTAGGCTAGCCTGGTTCCGCGTGGTTCTATGTCCCCTATACTAGGTTA-3′, and 5′-CAAGGGGATCCCTACGGAACCAGATCCGATTTTGG-3′, and inserted between the Nhe1 and BamH1 sites of pET-11d (the “pET-11d-GST vector”). MUC1/ECD was amplified by PCR using a set of primers as follows:
5′-TCTGGCCATGGGAGAAGGTACCATCAAT-3′, and
5′-AGCGCGCTAGCCCAGCCTGGCACCCCAGC-3′, and inserted between the Nco1 and Nhe1 sites of pET11d-GST vector (the “pET11d-GST-MUC1/ECD” vector).

The GST fusion protein was prepared by incubating logarithmically growing E. coli BL21(D3)pLysS cells transformed with pET11d-GST-MUC1/ECD or pET11d-GST with 0.1 mM isopropyl-β-D-thiogalatopyranoside for 6 hr at 25° C. Cells were pelleted and resuspended in PBS containing 20% sucrose, 5 mM MgCl₂, 0.5% NP-40, then sonicated. Debris was removed by centrifugation at 10,000×g for 30 min. at 4° C. The supernatant was applied to bulk glutathione sepharose 4B and incubated for 4 hr at 4° C. prior to washing and packing into a column. The fusion protein was eluted with 10 mM glutathione in 50 mM Tris-HCl, pH 9.5. The purified fusion protein was dialyzed with PBS.

Cytosolic fractions of cultured cells were prepared by harvesting ZR75-1 cells in PBS containing 40 mM EDTA. After washing with PBS, cells were resuspended with ice-cold homogenized buffer (20 mM Hepes-KOH, pH 7.5, 10 mM KCl, 1.5 mM MgCl₂, 1 mM dithiothreitol, 1 mM EGTA, 1 mM EDTA and protease inhibitors cocktail (Roche) and put on ice for 15 min. The suspension was homogenized with a Dounce homogenizer. The homogenate was centrifuged at 1.00×g for 5 min at 4° C. The supernatant was collected as a cytosol fraction and stored at 0° C.

The GST-MUC1/ECD fusion protein (1.8 mg) was immobilized on 400 μl of glutathione-sepharose 4B, which was packed into a column and equilibrated with buffer A (30 mM Tris-HCl, pH 7.5, 5 mM MgCl₂, 1 mM EDTA, and 1 mM dithiothreitol). The cytosolic fraction was first precleared by passing it through a glutathione-sepharose 4B column and was then loaded onto the GST-MUC1/ECD affinity column which was then washed twice with 2×10 ml of buffer A. The protein bound to the column was eluted by the addition of 2 ml of buffer B (buffer A containing 0.15 M NaCl), and fractions of 0.4 ml each were collected. The second and third fractions were mixed and loaded on sodium dodecyl sulfate-polyacyralmide gel electrophoresis (SDS-PAGE). As a control, the GST protein (1.5 mg) was immobilized on 400 μl of glutathione-sepharose 4B and the experiment performed as described above. The results demonstrated that MUC1/ECD binds to a 45 kDa protein. A similar experiment was performed with lysates of human MCF-7 breast carcinoma cells confirmed the binding of MUC1/ECD with a 45 kDa protein.

The 45 kDa from the GST-MUC1/ECD adsorbate was excised from the gel, dehydrated with acetonitrile, and then dehydrated with 100 mM ammonium bicarbonate. The gel pieces were then suspended in 12.5 ng/μl trypsin/50 mM ammonium bicarbonate. Digestion was carried out at 37° C. for 10-12 hr. The masses of the trypsin-digested peptides were analyzed by matrix assisted laser desorption/ionization-time of flight-mass spectroscopy (MALDI-TOF-MS) using a Voyager DE-PRO (Perceptive Biosystem Inc., Framingham, Mass.). Two related proteins, designated ML-1 and ML-2, were identified by mass fingerprinting. The sequences of ML-1 (SEQ ID NO: 3) and ML-2 (SEQ ID NO: 4) are as those previously disclosed for two neuregulin isoforms, NRG2 splice isoform 5 and NRG2 splice isoform 6 respectively.

### Example 4

### MUC1 as an Oncogene

A. MUC1 Supports Growth in Soft Agar

The expression of MUC1 was shown to be functionally significant regarding the exhibition of the malignant phenotype. Cell lines that stably express MUC1 were generated. HCT116 colon cancer cells were transfected with pIRES-puro2 vector or pIRES-puro2-MUC1 by lipofectamine and selected for puromycin-resistance. Studies were performed with human MUC1-negative HCT116 colon cancer cells with single clones that stably express either the empty vector (VCT116/V) or MUC1 (HCT116/MUC1). The HCT 116/V and HCT116/MUC1 cells were assayed for anchorage-independent growth in soft agar. Cells (1×10⁵/60 mm dish) were suspended in 0.33% agarose-containing DMEM medium supplemented with 10% FBS and layered over an agarose ptug (0.5% agarose in DMEM supplemented with 10% FBS). The cells were incubated to 4 weeks, during which time fresh medium was added to the plates every week. Colonies Larger than 70 μm in diameter were counted after 4 weeks. Expression of wild-type MUC1 was associated with a marked increase in the size and number of colonies compared to that obtained with HCT/116/V cells. These findings were confirmed by similar studies performed with SW480/V and SW480/MUC1 cells wherein MUC1 expression was again shown to support anchorage-independent growth of SW480 cells.

B. MUC1 Supports Human Tumor Formation in Nude Mice

To assess the effects of MUC1 on human tumor growth in vivo. Five to six week old athymic, Balbc/nu/nu mice (Taconic, Germantown, N.Y.) were injected subcutaneously in the right flank with 1×10⁶ HCT116/V or HCT 116/MUC1 cells. Tumors (4 mice/group) were measured twice a week. Tumor volumes were calculated by the following formula: ½(length×width²). Experiments were terminated when tumor volume exceeded 2 cm³. Measurements of tumor volume over time demonstrated little HCT116/V cell growth. By comparison there was a marked increase in the growth of HCT116/MUC1 tumors.

### Example 5

### MUC1 Expression is Induced by Oxidative and Genotoxic Stress

To determine whether MUC1 is induced in response to oxidative stress, MCF-7 cells were treated with hydrogen peroxide. Cell lysates were analyzed by immunoblotting with anti-DF3/MUC1 antibody. Lysates were prepared by suspending MCF-7 cells in lysis buffer (50 mM Tris, pH 7.6, 150 mM NaCl, 1 mM PMSF, 3 mM NaF, 1 mM sodium vandate, 1 mM DTT with protease inhibitors and either 1% NP-40 or 1% Brij-96) for 30 min. on ice. Lysates were cleared by centifugation and equal amounts of proteins were resolved by SDS-PAGE. Proteins were then transferred to nitrocellulose filters, blocked by incubation in 5% non-fat dry milk in PBS with 0.05% Tween-20 and probed with anti-MUC1 antibody (Pandey et al., 1995). Anti-actin was used as a control. The results demonstrated that MUC1 is rapidly and transiently induced in response to oxidative stress while there was no effect of hydrogen peroxide on levels of actin expression.

Similar studies were performed with genotoxic agents. The results demonstrate that treatment of MCF-7 cells with daunorubicin is associated with a rapid and transient induction of MUC1, and not actin, expression. The available evidence indicates that MUC1 is induced by diverse cytotoxic agents, including taxol, cisplatin and ionizing radiation.

### Example 6

### MUC1 Inhibits the Apoptotic Response to Oxidative and Genotoxic Stress

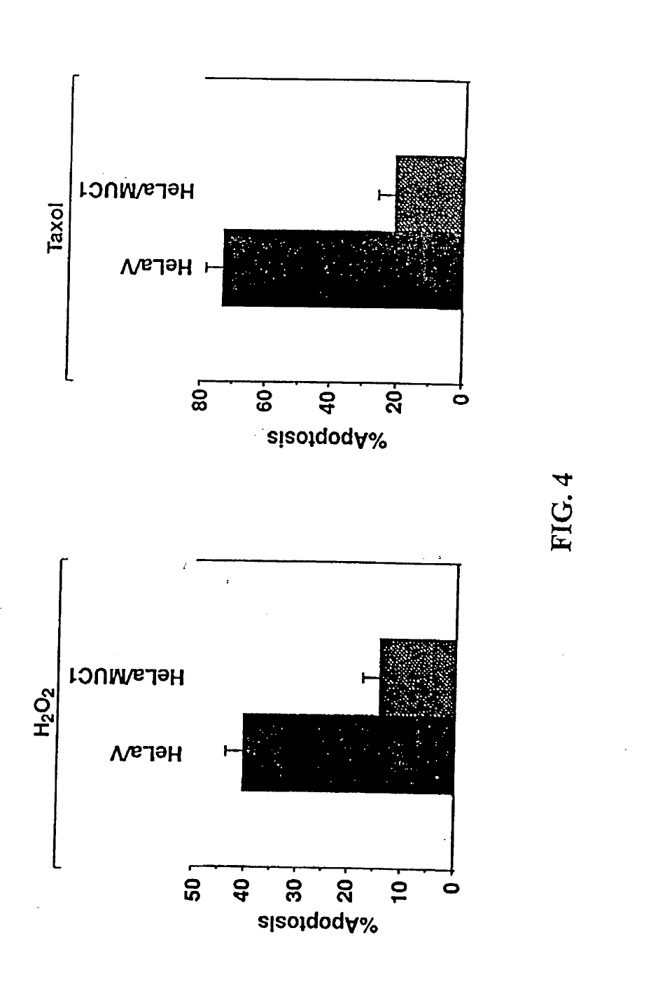

To determine whether MUC1 inhibits the apoptotic response to oxidative stress, HeLa cells stably expressing the empty vector or MUC1 were treated with 1 mM hydrogen peroxide for 1 hr. DNA content was assessed by staining ethanol-fixed cells with propidium iodide and monitoring by FACScan (Beckton Dickerson). Numbers of cells with sub-G1 DNA content were determined with a MODFIT LT program, (Verity Software House, Topsham, Me.) (Yuan et al., 1997). The cells were analyzed for induction of sub-G1 DNA by flow cytometry as a marker of apoptosis. As shown in FIG. 4, the results demonstrate that MUC1 inhibits the apoptotic response to oxidative stress.

The induction of apoptosis in cells treated with 0.01 mM taxol for 20 hr was assessed by measuring sub-G1 DNA content of HeLa cells expressing empty vector or MUC1. As shown in FIG. 4, as with oxidative stress, taxol-induced apoptosis was inhibited by MUC1 expression.

### Example 7

### Effect of NM-3 on MUC1 Expression

MCF-7 cells were treated with NM-3 (2-(8-hydroxy-6-methoxy-1-oxo-1 H-2-benzopyran-3-yl)propionic acid) at 100-400 μg/ml for 48 hr. DF3 antigen levels were visualized by immunoblot analysis with DF3 MAb (Kufe, U.S. Pat. No. 5,506,343, herein incorporated by reference).

A decrease in the intracellular levels of DF3 antigen of NM-3 treated cells relative to non-treated cells was observed. Similar results were found in ZR-75-1 and BT-20 cell lines. The NM-3 mediated decrease of intracellular DF3 antigen was shown to be both dose- and time dependent. To determine whether NM-3 impaired the extracellular localization of the MUC1 DF3 antigen, the antigen levels in cell culture medium supernatants and on MCF-7 cells after NM-3 treatment were investigated. The levels of DF3 antigen were reduced in both localizations. These findings suggest that NM-3 inhibits MUC1 protein expression.

Hybridization studies were performed to determine whether the effect of NM-3 on MUC1 -expression was detectable at the transcriptional level. A ³²P-labaelled DF3 DNA probe hybridized to two transcripts of 4.5 and 7.0 kb in total cellular RNA after NM-3 treatment on MCF-7 cells for 48 hr. The levels of both mRNA were decreased relative to controls wherein MCF-7 cells were incubated in the absence of NM-3. The same results were observed using ZR-75-1 and BT-20 cell lines. These findings suggest that DF3 expression is regulated at the transcriptional level after NM-3 treatment on those cell lines.

To determine whether NM-3 inhibits the expression of cellular surface proteins as well, the level of epidermal growth factor receptor (EGF-R) expression after NM-3 treatment was tested on MCF-7, ZR-75-1 and BT-20 cell lines. Compared to controls wherein cells were incubated in the absence of NM-3, there were no detectable changes in EGF-R expression after NM-3 treatment. These results indicate a selective effect of NM-3 on MUC1 expression without inhibition of surface molecular expression.

### Example 8

### Effect of CDDO and Analogs on MUC1 Expression

Cell Culture: Human MCF-7 breast carcinoma and HeLa cervical carcinoma cella (obtained form the ATCC, Manassas, Va.) were grown in DMEM (high glucose; Mediatech, Cellgrow) supplemented with 10% heat-inactivated fetal calf serum (FCS) and 2 mM L-glutamate. Celss were treated with oelanae triterpenoid 2-cyano-3,12-dioxoolean-1,9-diene-28-oic (CDDO) CDDO methyl ester (CDDO-Me), imadzole CDDO (CDDO-Im) or the 2-propyl-imadazole CDDO (CDDO-Pr-Im). In certain experiments, cells were treated with N-acetylcysteine (NAC; Sigma Chemical Co.) or glutathione (GSH; Sigma Chemical Co.).

Immunoblot analysis: Cells were lysed in ice-cold lysis buffer (20 mM Tris-HCl, pH 8.0, 150 mM NaCl, 1% Triton-X-100, 1 mM phenylsulfonyl fluoride, 1 mM DTT, 10 μgm/ml aprotinin) for 30 min. Lysates were cleared by centrifugation for 20 min. at 4° C. as described (Yin et al, 2001). Proteins were separated by SDS-PAGE, transferred to nitrocellulose and probed with anti-DF3/MUC1 (Kufe, U.S. Pat. No. 5,506,34) or anti-β-actin (Sigma). The antigen-antibody complexes were visualized by enhanced chemiluminescence (ECL; Amersham Life Sciences).

Reverse transcription-polymerase chain reaction (RT-PCR): Total cellular RNA was extracted in Triazol, dissolved in RNAase-free water and incubated for 10 min. at 55° C. MUC1-specific primers (5′-TCTACTCTGGTGCACAACGG-3′ and 3′-TTATATCGAGAGGCTGCTTCC-5′) were designed to span a region within the genomic DNA that contains two introns and result in amplification of a 489-bp fragment from RNA and a 783-bp fragment from genomic DNA. RNA-specific primers for human β-actin were used as a control. The RNA was reverse transcribed and amplified using SuperScript One-Step RT-PCR with Platinum Taq (GIBCO-BRL Gaithersburg, Md.). Amplified fragments were analyzed by electrophoresis in 2% agarose gels.

Measurement of ROS levels: Cells were incubated with 10 mM DCF-DA Sigma) for 15 min. at 37° C. to assess ROS-mediated oxidation of the fluorescent compound DCF (LeBel et al., 1992). Fluorescence of oxidized DCF was measured at an excitation wavelength of 480 nm and an emission wavelength of 525 nm using a flow cytometer (Beckton Dickerson, Lincoln Park, N.J.).

CDDO downregulates MUC1 expression: MUC1-positive cells were treated with CDDO at concentrations of 1, 3 and 5 μM. Immunoblot analysis of cell lysates with anti-MUC1 demonstrated little if any effect of 1 or 3 μM CDDO at 24 hr while exposure to 5 μM CDDO was associated with decreased levels of MUC1 protein. By contrast, 5 μM CDDO had no apparent effect on β-actin gene expression. Studies with 1 μM CDDO-Me also demonstrated downregulation of MUC1 expression that was detectable at 12 hr and nearly complete at 24 hr. Other substitutents of the carboxyl group at C-17 of CDDO were effective in downregulating MUC1 expression. For example, both the imadazole derivative CDDO-Im and 2-propyl-imadazole derivative CDDO-Pr-Im decreased levels of MUC1 protein at a concentrations of 0.8 μM.

To determine whether MUC1 expression is decreased at the mRNA level , RT-PCR was performed on MCF-7 cells treated with CDDO and the derivatives. The results demonstrate that CDDO treatment is associated with a decrease in MUC1, but not β-actin, transcripts. Similar findings were obtained with CDDO-Me, CDDO-Im and CDDO-Pr-Im. Whereas CDDO induces apoptosis of diverse cell types, we assessed viability of MCF-7 cells under the same experimental conditions. The results demonstrate that CDDO and its derivatives have little if any effect on MCF-7 cell viability at 24 hours of exposure. By contrast, longer periods of treatment (48 and 72 hr) were associated with detectable decrease in viability.

Studies were also performed with MUC1 -positive HeLa cervical carcinoma cells. The results demonstrate that CDDO, CDDO-Me, CDDO-Im and CDDO-Pr-Im decrease MUC1 expression at concentrations below decrease expression at concentrations below 1 μM. RT-PCR studies with HeLa cells also demonstrated that these agents decrease MUC1 transcripts.

CDDO increase intracellular ROS levels: Cells were incubated with DCF-DA and ROS-mediated oxidation of the fluorochrome was assessed by flow cytrometry. Compared to control MCF-7 cells, treatment with CDDO was associated with detectable increases in ROS. As a control, cells were incubated with NAC, a scavenger of ROS. NAC pretreatment was associated with abrogation of CDDO-induced ROS production. Similar findings were obtained with CDDO-Me, CDDO-Im and CDDO-Pr-Im. The effects of CDDO and the derivatives on ROS were comparable to that found following exposure to 0.1 mM H₂O₂.

CDDO-induced downregulation of MUC1 is blocked by antioxidants: To determine whether the increases in ROS contribute to the downregulation of MUC1 expression, cells were pretreated with NAC and then incubated with CDDO or one of the derivatives. The results demonstrate that CDDO-, CDDO-Me-, CDDO-Im- and CDDO-Pr-Im-induced decreases in MUC1 are blocked by NAC. Treatment of cells with the antioxidant GSH also blocked the effects of CDDO and the derivatives on MUC1 expression. In concert with these results, treatment of cells with 0.1 mM H₂O₂ was associated with downregulation of MUC1 and this response was reversed with NAC. These finding support a model in which CDDO and its derivatives decrease MUC1 expression by a ROS-mediated mechanism.

### Example 9

### Effect of siRNA on MUC1 Expression

Cell Culture: Human MCF-7 breast cancer cells were maintained in DMEM supplemented with 10% heat inactivated fetal bovine serum (FBS) and 1% penicillin-streptomycin mixture. Human A549 non-small cell lung cancer cells were maintained in RPMI1640 medium supplemented with 10% FBS and 1% penicillin-streptomycin mixture.

siRNAs: Five different double stranded anti-MUC1 siRNAs were designed: one against the target sequence SEQ ID NO: 63, i.e., AAGGGGGTTTTCTGGGCCTCT, with sense and antisense strands as shown in SEQ ID NO: 64 and SEQ ID NO: 65 respectively (hereinafter siRNA #1); one against the target sequence SEQ ID NO: 42, i.e., AAGGTACCATCAATGTCCACG, with sense and antisense strands as shown in SEQ ID NO: 43 and SEQ ID NO: 44 respectively (hereinafter siRNA #2); one against the target sequence SEQ ID NO: 21, i.e., AAAAGGAGACTTCGGCTACCC, with sense and antisense strands as shown in SEQ ID NO: 22 and SEQ ID NO: 23 respectively (hereinafter siRNA #3); one against the target sequence SEQ ID NO: 66, i.e., AAGTTCAGTGCCCAGCTCTAC, with sense and antisense strands as shown by SEQ ID NO: 67 and SEQ ID NO: 68 respectively (hereinafter siRNA #4); and one against target sequence SEQ ID NO: 69, i.e., AAGGTTTCTGCAGGTAACGGT, with sense and antisense strands as shown by SEQ ID NO: 70 and SEQ ID NO: 71 respectively (hereinafter siRNA #5). A control siRNA was designed against the scrambled target SEQ ID NO: 72 with sense and antisense strands as shown by SEQ ID NO: 73 and SEQ ID NO: 74 respectively. Each RNA sense oligonucleotide (50 μM) was combined with equal volume of antisense oligonucleotide (50 μM) in an annealing buffer (20 mM KCl, 6 mM HEPES-KOH, pH 7.5, 0.2 mM MgCl₂). The solution was incubated for 1 min at 90° C. to denature secondary structure, and placed at 37° C. for 1 hour to anneal the complementary strands. The subsequent double stranded RNAs were used as the anti-MUC1 siRNA.

Transfection: Cells (1-3×10⁵ cells/well) were plated in 6-well plates in growth medium without antibiotics, and incubated overnight. After washing cells once with Opti-MEM I (Invitrogen), 800 μL of fresh Opti-MEM I and 200 μL of the Reagent (Invitrogen) were added to the cultured cells. Four hours later, 300 μL of FBS was added to the cultured cells.

Western Blot Analysis: Cell were passed a day before transfection. MUC1 siRNA (0.2 micromolar) was added with oligofectamine to cells in 0% FBS. Four hours later serum was added to a final concentration of 10%. After various time intervals (48 h, 72 h and 96 h), cells were washed and total cell lysates were prepared. Proteins were separated from cell lysates by SDS-PAGE, transferred to nitrocellulose and analyzed by immunoblotting with anti-DF3 antibodies.

RT-PCR: Total RNA was extracted and purified from cultured cells using RNAzol B according the manufacturer's instructions. The RNA was quantified by determining absorbance at 260 nm. One μg of total RNA from each sample was reverse transcribed into cDNA using Thermoscript reverse transcriptase (Invitrogen) in a volume of 20 μL. The cDNA product was amplified by PCR using Platinum Taq DNA polymerase (Invitrogen) and specific primers for MUC1 and beta-actin as an internal control. The sequences of primers used were as follows;
MUC1 (forward, 5′-GGTACCATCAATGTCCACG-3′; reverse, 5′-CTACAAGTTGGCAGAAGTGG-3′) and beta-actin (forward, 5′-ATCATGTTTGAGACCTTCAA-3′; reverse, 5′-CATCTCTTGCTCGAAGTCCA-3′).

The reaction was performed using the following program: 94° C. for 2 min, 29 cycles (25 cycles for beta-actin) at 94° C. for 2 min, 48° C. for 1 min, 72° C. for 30s and then additional extension step of 72° C. for 10 min. The PCR products were separated on a 2% agarose gel, stained with ethidium bromide and photographed under UV light.

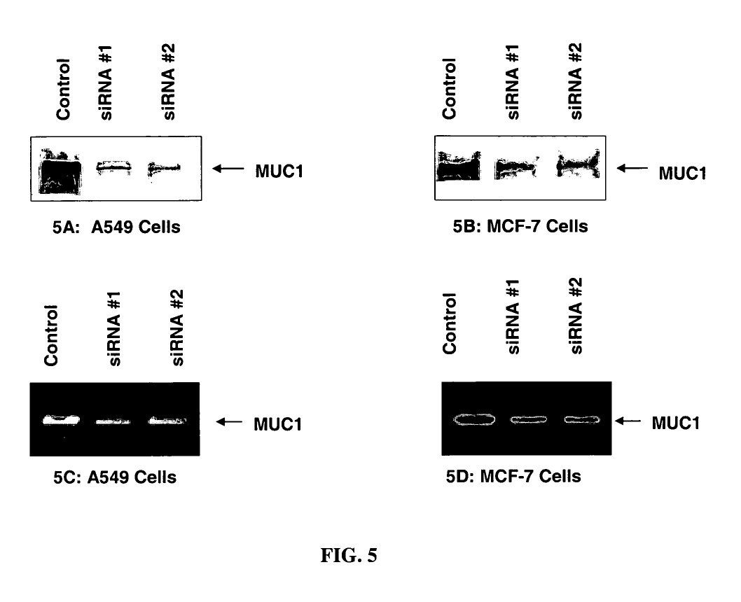

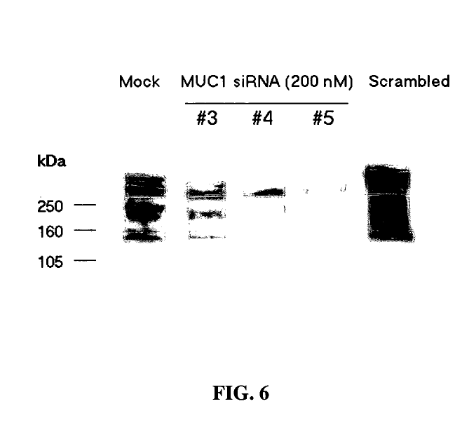

SIRNA downregulates MUC1 expression: As shown in FIG. 5, both double stranded siRNA's #1 and #2 downregulate MUC1 expression at both the protein level, as shown by the immunoblot results, and at the RNA level, as shown by the RT-PCR results, in both MCF-7 and A549 cells. FIG. 6 shows the downregulation of MUC1 expression by siRNAs #3, #4 and #5 in MCF-7 cells at the protein level by immunoblot.

### Example 10

### Combination of MUC1 Directed siRNA and Cisplatin (CDDP) on Human Non-small Cell Lung Cancer A549 cells

Cell culture: Human non-small cell lung cancer A549 cells were grown in RPMI1640 medium supplemented with 10% fetal bovine serum (FBS), 100 units/ml of penicillin and 100 μg/ml of streptomycin, respectively.

Transfection with siRNA: Cells (3×10⁵ cells/well) were plated in 6-well plates in growth medium without antibiotics, and incubated overnight. Cells were then transfected with MUC1 siRNAs, siRNA #1, i.e., one against the target sequence SEQ ID NO: 63, i.e., AAGGGGGTTTTCTGGGCCTCT with sense and antisense strands as shown in SEQ ID NO: 64 and SEQ ID NO: 65 respectively; and siRNA #2, i.e., one against the target sequence SEQ ID NO: 42, i.e., AAGGTACCATCAATGTCCACG with sense and antisense strands as shown in SEQ ID NO: 43 and SEQ ID NO: 44 respectively, and one control siRNA directed towards the non-specific gene target AAGCGCGCTTTGTAGGATTCG using Oligofectamine reagent (Invitrogen) according to the manufacturer's instructions. After 24 hr of transfection, the medium was changed to complete medium containing antibiotics.

Treatment of Cells with cisplatinum (CDDP): At 72 hr post transfection of siRNAs, 10 μM CDDP was added to the cells in fresh culture medium. After 48 hr of CDDP exposure cells were trypsinized and analyzed for MUC1 expression and apoptosis as described below.

Apoptosis assay.: A549 cells were initially stained for MUC1 expression using the DF3 monoclonal antibody (Kufe, U.S. Pat. No. 5,506,343, herein incorporated by reference). Cells (approx. 6×10⁵ per well) were trypsinized for approx. 5 minutes with 0.5 ml per well (of 6 well plate), of a 0.05% trypsin solution, after which 1.0 ml of 10% FBS containing DMEM medium was added to inhibit trypsin activity. Two wells per treatment group were combined to generate the sample for staining. Cells were pelleted by centrifugation at approx. 300×g, then resuspended in 2 ml staining buffer (PBS with 1% BSA, and 0.1% sodium azide). Cells were transferred to a 3 ml polystyrene tube and pelleted again. The cell pellet was resuspended in 200 μl of staining buffer containing DF3 monoclonal antibody at a concentration of 500 ng/ml. Binding of the DF3 antibody was carried out at 4° C. for 1 hour with gentle rocking of the tubes in an upright rack. The amount of DF3 staining was then measured using a biotinylated goat anti-mouse secondary antibody (diluted 1:200 in staining buffer) and streptavidin-conjugated phycoerythrin (diluted 1:50 in staining buffer). After fluorescent staining for MUC1 expression, apoptosis was determined by staining cells with Annexin V-FITC and propidium iodide (PI). Stained cells were washed once with binding buffer [10 mM HEPES/NaOH (pH 7.4), 140 mM NaCl, 2.5 mM CaCl2], and then resuspended in 200 μl of binding buffer containing 0.5 μg/ml annexin V-FITC (Clontech Laboratories) and 1.25 μg/ml PI (Clontech). Staining of apoptotic cells was allowed to proceed at room temperature for 20 minutes with gentle shaking. Cells were again pelleted, washed with 1 ml of binding buffer and resuspended in 0.5 ml binding buffer for analysis of apoptosis. Using a flow cytometer, (EPICS XL-MCL, Coulter Corp), the amount of MUC1 expressing cells were determined using the FL2 detector, annexin V-FITC staining was measured in the FL1 detector and propidium iodide staining was measured in the FL3 detector. Overlapping signal or bleed over between the detectors was compensated for using control cells stained with only one of each of the fluorescent dyes.

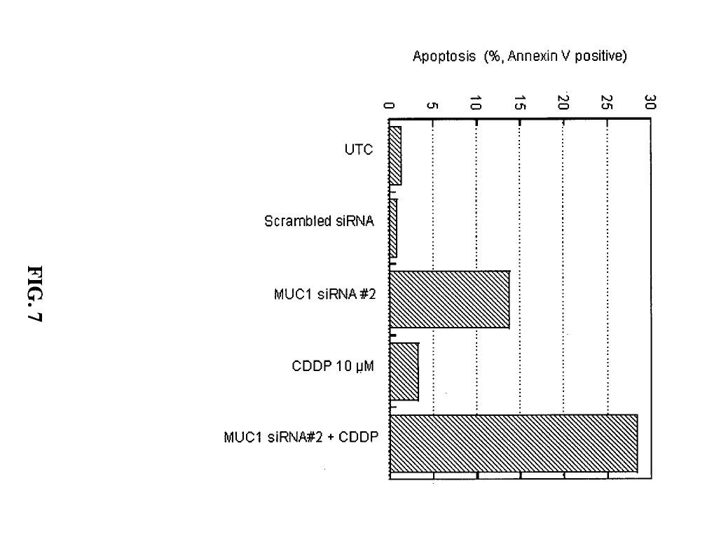

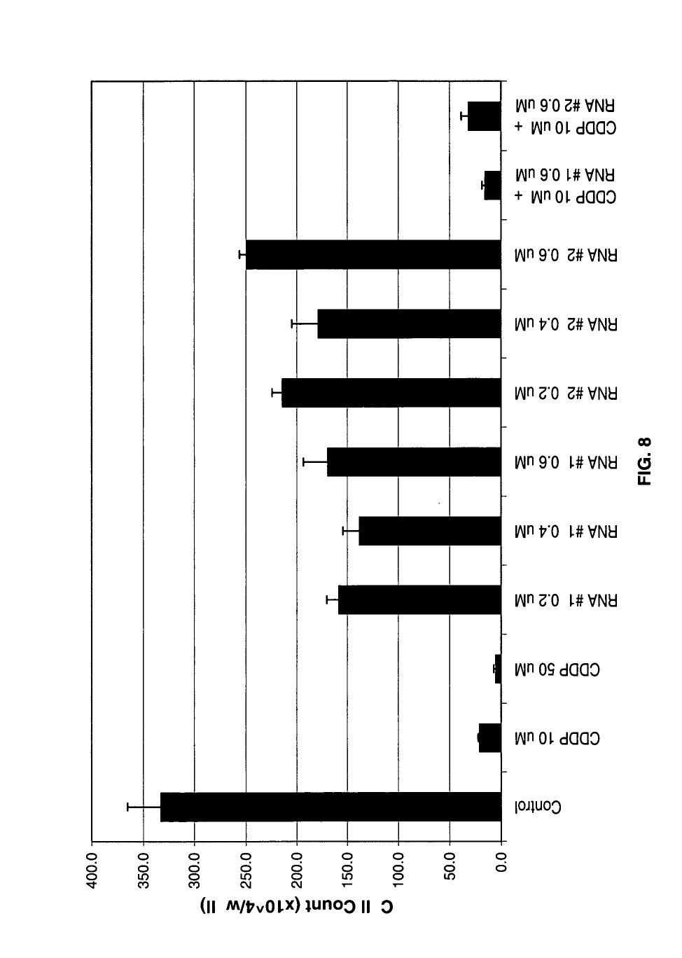

MUC1 siRNAs Enhance the Efficacy of CDDP: FIG. 7 shows that MUC1 siRNA #2 transfected A549 cells exhibited an increase in the percentage of apoptotic cells relative to controls and also more than additively enhances the percentage of apopotic cells when combined with CDDP exposure. FIG. 8 also shows that transfection of both siRNA #1 and siRNA #2 decreased the proliferation of A549 cells.

### Example 11

### Construction of Plasmids that Expresses MUC1 siRNA

Plasmids expressing hairpin MUC1 siRNA (targeted sequence, 5′-AAGGTACCATCAATGTCCACG-3′) were constructed by using expression vectors with different promotors, pSilencer (U6 promoter, Ambion) and pSuper (H1 promoter, OligoEngine). To insert the target sequence that encodes the MUC1 siRNA, the following DNA oligos were synthesized, annealed and cloned into the Apa I and EcoR I site of pSilencer (pSilencer/MUC1siRNA) and the Bgl II and Hind III site of pSuper (pSuper/MUC1siRNA), respectively.

pSilencer,
5′-GGTACCATCAATGTCCACGTTCAAGAGACGTGGACATTGATGGTACC
TTTTTT-3′ and
5′-AATTAAAAAAGGTACCATCAATGTCCACGTCTCTTGAACGTGGACAT
TGATGGTACCGGCC-3′
pSuper,
5′-GATCCCCGGTACCATCAATGTCCACGTTCAAGAGACGTGGACATTGA
TGGTACCTTTTTGGAAA-3′ and
5′-AGCTTTTCCAAAAAGGTACCATCAATGTCCACGTCTCTTGAACGTGG
ACATTGATGGTACCGGG-3′

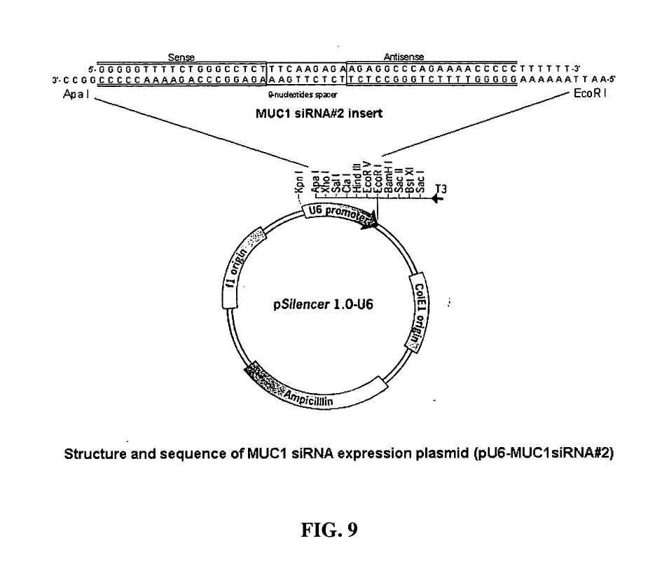

The structure of an expression plasmid containing the anti-MUC1 siRNA #2 is shown in FIG. 9.

Transfection with MUC1 siRNA expression plasmids. Human breast cancer MCF-7 and ZR-75-1 cells were transfected with pSilencer/MUC1siRNA and pSuper/MUC1siRNA by using Lipofectamine 2000 (Invitrogen) according to the manufacturer's instructions. Human multiple myeloma RPMI8226 and U266 cells were also transfected by electroporation of 4×10⁶ cells with 20 μg pSilencer/MUC1siRNA or 30 μg pSuper/MUC1siRNA in 0.4 ml of RMPI1640 medium. Electroporation was performed by the Gene Pulser (Bio-Rad Laboratories) at 0.22 kV and 960 μF capacitance. To obtain the stable transfectants, cells were co-transfected with a plasmid carrying a neomycin-resistance gene (pClneo, Promega), followed by selection in medium containing 400-600 μg/ml G418.

MCF-7 cells transfected with pSilencer/MUC1siRNA or vector were harvested at 48 hr post transfection and subjected to Western blot analysis with anti-MUC1antibody (DF3). The results showed down-regulation of MUC1 protein expression.

### Example 12

### Generation of Monoclonal Antibody IPB-01

A chimeric protein containing the mouse Fc region and the extracellular domain of MUC1-Y was prepared for use as an antigen. Full-length cDNA of MUC1-Y (Baruch et al., 1997) was constructed in three steps of PCR. In the first PCR, cDNA coding for MUC1 signal peptide was made with the MUC1 primers:

(5′-CTAGCTAGVATGACACCGGGCACCC-AGTC-3′, and
5′-GGAATTAAAAGCATTCTTCTCAGTAG-3′.

Then the primers:

5′-AATGCTTTTAATTCCTCTCTG-3′, and
5′-CTTAAGCTACAAGTTGGCAGAAGT-3′,

were used for the second PCR to produce cDNA of MUC1-Y without signal peptide. The mixture of first and second PCR products was taken as a template, and the full-length of MUC1-Y cDNA was amplified in the third PCR with the primers:

5′CTAGCTAGC-ATGACACCGGGCACCCAGTC-3′, and
5′-CTTAAGCTACAAGTTGGCAGAAGT-3′.

After digestion of both MUC1-Y cDNA and pIRESpuro2 vector (Clontech Lab., Inc) with Nhe I and Aft II, DNA fragments were separated on 1.2% agarose gel. MUC1-Y DNA was purified and ligated into pIRESpuro2 vector. The construct was confirmed by both enzymes digestion and DNA sequencing.

The cDNA sequences of mouse IgG1 Fc fragment and human IgG2a Fc fragment was cloned in to expression vector, pEF6/V5.His (Invitrogen Cat #V96120), resulting in pEF6/V5.His-mFc and pEF6/V5.His-hFc. The cDNA of the extracellular domain of MUC1-Y (MUC1-Yex) was amplified by PCR using the primers:

MUC1/Yex-N-NheI:
5′-CCC ACC GCT AGC ACC ACC ACC ATG ACA CCG-3′,
MUC1/Yex-C-HindIII:
5′-CCA GCC AAG CTT CCC AGC CCC AGA CTG GGC-3′,

and cloned, in frame, upstream of mFc sequence in pEF6/V5.His-mFc resulting in pEF6/V5.His-MUC1/Yex-mFc. The expression plasmid was confirmed by DNA sequencing.

The expression plasmid was transfected into 293 or CHO K1 cells by lipofectamine. For transient transfection, 293 cells were cultured 72 hr after transfection, and the supernatant was collected. Chimeric protein was purified by chromatography using protein A column. For stable transfection, transfected CHO K1 cells were selected by antibiotics, and single clones were selected and expanded. Cell culture supernatant was passed through a protein A column to purify this chimeric protein.

The chimeric protein as expressed contained the MUC1-Y extracellular domain plus the N-terminal sequence (SEQ ID NO: 105):

MTPGTQSPFFLLLLLTVLTATTAPKPATVVTGSGHASSTPGGEKETSATQ
RSSVPSSTEKNAFNSSLEDPSTDYYQELQRDISEMFLQIYKQGGFLGLSN
IKFRPGSVVVQLTLAFREGTINVHDVETQFNQYKTEAASRYNLTISDVSV
SDVPFPFSAQSGAG.

Upon secretion from the cell, the N-terminal sequence was cleaved resulting in a mFc chimeric protein containing the 102 amino acid MUC1-Y extracellular sequence (SEQ ID NO: 106):

FNSSLEDPSTDYYQELQRDISEMFLQIYKQGGFLGLSNIKFRPGSVVVQLTLAFREGTINVHDVETQFN
QYKTEAASRYNLTISDVSVSDVPFPFSAQSGAG.

Purified MUC1-Y-mFC protein used to immunize BALB/C mice according to the following schedule:

| | |
|---|---|
| Day 0: | 50 μg antigen/animal in Freund's Complete Adjuvant |
| Day 14: | 40 μg/animal in Freund's Incomplete Adjuvant |
| Day 35: | 40 μg/animal in Freund's Incomplete Adjuvant |
| Day 45: | Test bleed |
| Day 56: | Final boost 15 μg IV and 15 μg/IP in PBS |
| Day 59: | Fusion |

The splenocytes of the mice with high titer against antigen were isolated and fused to BALB/C derived myeloma cell lines, X63-Ag8.653 or P3, in the presence of PEG. Hybridomas were selected in HAT media. Selected hybridomas were tested against the antigen by solid phase ELISA and positive hybridomas were cloned by serial dilution and limited dilution. After at least two rounds of cloning and testing, positive clones were expanded and tested in solid phase ELISA, flow cytometry, epitope mapping and growth assays.

For the solid phase ELISA assay, Dynex Immulon 1B plates were coated overnight at 4° C. with 100 μl MUC1-Yex-mFc solution containing 1 μg/ml of protein in Coating Buffer (0.1M carbonate/bicarbonate buffer, pH 9.6), and blocked with 200 μl/well of PBS (Ca²⁺/Mg²⁺Free) containing 4% Non Fat Dry Milk and 0.05% Tween-20. Hybridoma supernatants were then added followed by incubation at room temperature for 2 hr. The plates were washed 4 times with PBS containing 0.05% Tween-20 and incubated at room temperature for 1 hr with 100 μl rabbit HRP-labeled anti-mouse antibody at a 1:10,000 dilution. After washing, 100 μl of TMB substrate was added to each well and incubated for 10 minutes at room temperature. The reaction was stopped by addition of 100 μl stop solution. The plates were read at 450 nm.

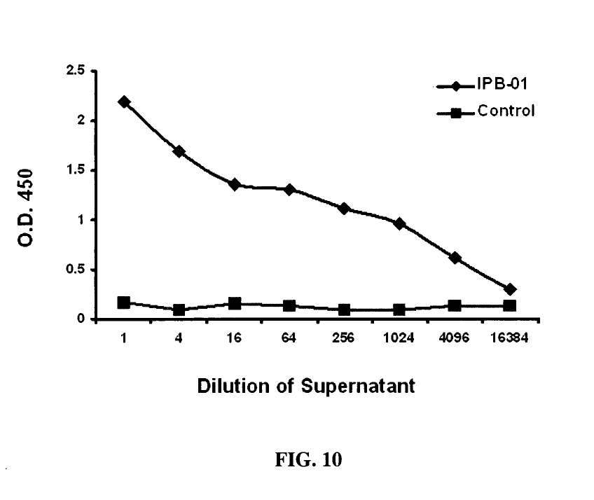

An antibody designated IPB-01 was peptide mapped to a sequence designated as A-1 (SEQ ID NO: 107) FNSSLEDPSTDYYQELQRD. The binding of IPB-01 to immobilized MUC1-Yex-mFc is shown in FIG. 10. The binding to MUC1 expressed on the cell surface was determined by flow cytometry. HTC cells were transfected with the expression plasmids for MUC1/Y or the empty expression vector by Lipofectamine (Invitrogen Cat #18324). Transfectants were selected for antibiotics resistance for 2 weeks. Single clones were then selected and expended. Expression of MUC1-Y was confirmed by Western analyses and flow cytometry for specific binding of antibodies against the tandem repeats of MUC1-Y (DF3E). In addition, MCF-7 human breast cancer cells that express full length MUC1 endogenously were also used for the flow analyses. For the determination of the binding of IPB-01, cells were incubated with IPB-01 or isotype control antibody for 2 hr at 4° C. Cells were washed and incubated in FACS buffer (PBS containing 0.1% Bsa and 0.1% NaN₃), containing biotin-conjugated anti-mouse antibody for 1 hr at 40° C. After incubation, cells were washed and incubated with PE-conjugated streptavidin and analyzed using a Beckman Coulter flow cytometer. IPB-01 was determined to bind to MUC1-Y but not full length MUC1 as expressed on the cell surface. This specificity of binding was verified by immunofluorescent microscopy. Cells expressing full length MUC1 or MUC1-Y were grown in multi-chamber slides and incubated with IPB-01 for 1 hr at room temperature. Cells were washed and incubated with FITC-conjugated anti-mouse IgG and the binding of IPB-01 to cells was analyzed by fluorescence microscopy. IPB-01 was observed to bind only to MUC1-Y expressing cells.

Monoclonal antibody IPB-01 may suitably be used for diagnostic uses, e.g., directly or indirectly labeled by a suitable fluorophore or chromophore, or as a therapeutic agent, e.g., as when attached to a toxin or radiolabel.

As such, IPB-01 directed saporin toxin induced cell death was investigated using the non-MUC1 expressing colon carcinoma cell line HCT116 wherein a daughter cell line was transfected with a MUC1-Y gene in the “pIRES-puro2” vector or an empty vector.

Appropriately transfected HCT116 cells were plated at a concentration of 4000 cells per well in a volume of 80 μl into 96 well tissue-culture treated plates. Each cell line was plated onto a separate plate. All cell lines were grown in DMEM medium containing 10% fetal bovine serum.

Added primary antibody (IPB-01) (3 μg/ml) and secondary antibodies, saporin tagged goat anti-mouse IgG (S-GAM) or saporin tagged normal goat IgG (S-G) (5 or 0.5 μg/ml) to groups in quadruplicate, i.e. four welts per treatment group. Both primary and secondary antibodies were added in 10 μl aliquots. The cells we incubated with the treatment antibodies for 48 hrs (1^{st} experiment) or 72 hours (2^{nd} experiment). At the end of the incubation period, 10 μl of MTT reagent (5 mg/ml in PBS) were added to each of the treatment wells of the 96 well plate as well as three wells containing no cells. Plates were placed back into the 37 degree incubator for 4 hours. At the end of the 4 hours incubation, 100 μl of a 10% SDS/0.01M HCl solution was added to all of the wells of the plate to dissolve the formazan crystals. After an overnight incubation at 37° C. the plates were allowed to cool to room temperature then the optical density at 562 nm and 650 nm of the wells were determined using a plate based spectrophotometer. The 650 nm reading was subtracted from the 562 nm reading to remove absorption due to the unconverted MTT reagent still in the well. The OD 562-650 readings of each group were averaged and divided by the average reading of the no antibody control group. This ratio multiplied by 100 yielded a % control viability value. The percent control viability value was subtracted from 100% to yield a % inhibition. Results from 1^{st} experiment are summarized in Table 2 and results from 2^{nd} experiment and HCT116/Vector cells are summarized in Table 3.

TABLE 2

| Treatment | HCT/MUC1-Y^{†} | HCT/Vector^{†} |
|---|---|---|
| S-G (5 μg/ml) | 10.15 | 10.70 |
| S-GAM (5 μg/ml) | 6.27 | 12.79 |
| IPB-01 + S-G (5 μg/ml) | 11.04 | 14.62 |
| IPB-01 + S-G (0.5 μg/ml) | 7.16 | 10.97 |
| IPB-01 + S-GAM (5 μg/ml) | 25.67 | 11.49 |
| IPB-01 + S-GAM | 17.61 | 14.36 |
| (0.5 μg/ml) | | |
| Medium alone added | 0.00 | 0.00 |
| No additional medium | 5.37 | 9.14 |
| added | | |

TABLE 3

| Treatment | HCT/MUC1-Y^{†} | HCT/Vector^{†} |
|---|---|---|
| S-G (5 μg/ml) | 11.73 | 19.49 |
| S-GAM (5 μg/ml) | 6.92 | 7.95 |
| IPB-01 | 4.81 | 11.67 |
| IPB-01 + S-G (0.5 μg/ml) | 10.00 | 13.08 |
| IPB-01 + S-GAM (5 μg/ml) | 46.15 | 11.67 |
| Medium alone added | 0.00 | 0.00 |
| No additional medium | 3.27 | −1.54 |
| added | | |

^{†}Inhibition calculated as 100- % control viability.

### Example 13

### Generation of Monoclonal Antibody IPB-02

The DNA sequence representing the 100 amino acid MUC1-ECD region (SEQ ID NO: 108):

SSLEDPSTDYYQELQRDISEMFLQIYKQGGFLGLSNIKFRPGSVVVQLTLAFREGTINVHDVETQFNQY
KTEAASRYNLTISDVSVSDVPFPFSAQSGAG

was amplified by RT-PCR and cloned into His-tagged expression plasmid, pET-28a (Novagen, Cat #69864-3). The plasmid was introduced to E. coli bacterial strain, BL21(DE3) (Novagen Cat #69387) by heat shock transformation. Large scale of bacterial cells was cultured and production of His-ECD was induced by IPTG at 1 mM for 5-6 hr. Bacterial cells were collected by centrifugation and lysed with Benzonase Nuclease (Novagen Cat #70664-3) and lysozyme. The inclusion bodies were purified and His-ECD protein was purified, under denatured condition, using Ni-column chromatography. Purified protein was then refolded by serial dialyses to exclude the denaturing agent, urea.

His-ECD was conjugated to KLH and used to immunize BALB/C mice according to the following schedule:

| | |
|---|---|
| Day 0: | 50 μg antigen/animal in Freund's Complete Adjuvant |
| Day 14: | 40 μg/animal in Freund's Incomplete Adjuvant |
| Day 35: | 40 μg/animal in Freund's Incomplete Adjuvant |
| Day 45: | Test bleed |
| Day 56: | Final boost 15 μg IV and 15 μg/IP in PBS |
| Day 59: | Fusion |

The splenocytes of the mice with high titer against antigen were isolated and fused to BALB/C derived myeloma cell lines, X63-Ag8.653 or P3, in the presence of PEG. Hybridomas were selected in HAT media. Selected hybridomas were tested against the antigen by solid phase ELISA and positive hybridomas were cloned by serial dilution and limited dilution. After at least two rounds of cloning and testing, positive clones were expanded and tested in solid phase ELISA, flow cytometry, epitope mapping and growth assays.

For the solid phase ELISA assay, Dynex Immulon 1B plates were coated overnight at 4° C. with 100 μl His-ECD solution containing 1 μg/ml of protein in Coating Buffer (0.1M carbonate/bicarbonate buffer, pH 9.6), and blocked with 200 μl/well of PBS (Ca++/Mg++ Free) containing 4% Non Fat Dry Milk and 0.05% Tween-20. Hybridoma supernatants were then added followed by incubation at room temperature for 2 hr. The plates were washed 4 times with PBS containing 0.05% Tween-20 and incubated at room temperature for 1 hr with 100 μl rabbit HRP-labeled anti-mouse antibody at a 1:10,000 dilution. After washing, 100 μl of TMB substrate was added to each well and incubated for 10 minutes at room temperature. The reaction was stopped by addition of 100 μl stop solution. The plates were read at 450 nm.

An antibody designated IPB-02 was peptide mapped to a sequence designated as P-0 (SEQ ID NO: 109) SNIKFRPGSVVVQLTLAFRE. The binding of IPB-02 to immobilized His-ECD is shown in FIG. 10. The binding to MUC1 expressed on the cell surface was determined by flow cytometry. HTC cells were transfected with the expression plasmids for MUC1/Y or the empty expression vector by Lipofectamine (Invitrogen Cat #18324). Transfectants were selected for antibiotics resistance for 2 weeks. Single clones were then selected and expended. Expression of MUC1-Y was confirmed by Western analyses and flow cytometry for specific binding of antibodies against the tandem repeats of MUC1-Y (DF3E). In addition, MCF-7 human breast cancer cells that express full length MUC1 endogenously were also used for the flow analyses. For the determination of the binding of IPB-02, cells were incubated with IPB-02 or isotype control antibody for 2 hr at 40° C. Cells were washed and incubated in FACS buffer (PBS containing 0.1% BSA and 0.1% NaN₃), containing biotin-conjugated anti-mouse antibody for 1 hr at 40° C. After incubation, cells were washed and incubated with PE-conjugated streptavidin and analyzed using a Beckman Coulter flow cytometer. IPB-02 was determined to bind to both full length MUC1 and MUC1-Y expressed on the cell surface.

Monoclonal antibody IPB-02 may suitably be used for diagnostic uses, e.g., directly or indirectly labeled by a suitable fluorophore or chromophore, or as a therapeutic agent, e.g., as when attached to a toxin or radiolabel.

### Example 14

### In Vitro and In Vivo Down-regulation of MUC1 and Sensitization to Chemotherapeutic Agents by Transduction of Cancer Cells with a Reteroviral Vector

Cell culture: Human HCT116 cells (ATCC, Manassas, Va.) were cultured in Dulbecco's modified Eagle's medium/F12 with 10% heat-inactivated fetal calf serum (FCS), 100 units/ml penicillin, 100 μg/ml streptomycin and 2 mM L-glutamate. Human ZR-75-1 breast carcinoma cells (ATCC, Manassas, Va.) were grown in RPMI 1640 medium containing 10% FCS, antibiotics and 2 mM L-glutamate. HCT cells were transfected with pIRES-puro2 or pIRESpuro2-MUC1 as described (Li et al., 2001a).

Subcellular fractionation: Purified mitochondria were prepared as described (Kumar et at., 2003). Cell membranes were purified from supernatants after sedimentation of mitochondria as described (Kharbanda et at., 1996).

Analysis of mitochondrial transmembrane potential: Cells were incubated with 0.5 nM 3,3-dihexyloxocarbocyanine iodide (DiOC₆[3]; Molecular Probes) for 30 min and analyzed by flow cytometry as described (Shapiro, 2000).

Immunoblot analysis: Lysates were prepared from cells as described (Li et al., 2001]. Equal amounts of protein were separated by SDS-PAGE and transferred with anti-cytochrome c (BD PharMingen, San Diego, Calif.). The immunocomplexes were detected with horseradish peroxidase-conjugated secondary antibodies and enhanced chemiluminescence (ECL, Amersham Biosciences, Piscataway, N.J.). Intensity of the signals was determined by densiometric scanning.

Apoptosis assay: Apoptotic cells were quantified by analysis of sub-G1 DNA. Cells were harvested, washed with PBS, fixed with 80% ethanol, and incubated in PBS containing 20 ng/ml RNase (Roche) for 60 min at 37° C. Cells were stained with 40 μg/ml propidium iodide (Sigma) for 30 min at room temperature in the dark. DNA content was analyzed by flow cytometry (EPICS XL-MCL, Coulter Corp.).

Generation of retroviral vectors expressing MUC1siRNA: Oligonucleotides of siRNA were designed that contained a sense strand or 19 nucleotide sequences of MUC1 (based on MUC1 DNA target SEQ ID NO: 42) followed by a short spacer (GAGTACTG), the reverse complement of the sense strand, and five thymidines as an RNA polymerase III transcriptional stop signal. Forward oligonucleotides for MUC1 were TCGAGGGTACCATCAATGTCCACGGAGTACTGCGTGGACATTGATGGTACCTTTTT (SEQ ID NO: 110) including a Xho I cleavage site and the reverse CTAGAAAAAGGTACCATCAATGTCCACGCAGTACTCCGTGGACATTGATGGTACCC (SEQ ID NO: 111) including a Xba I site. Oligos were annealed and cloned into the Xho I-Xba I site of the pSuppressorRetro vector (Imgenex Colo., San Diego, Calif.). 293T cells were cotransfected with a plasmid expressing MUC1siRNA and pCL Ampho virus using Fugene (Roche., Indianapolis). The supernatant was collected after 48 h for infection of target cells.

Results: Infection of ZR-75-1 breast carcinoma cells with the MUC1siRNA reterovirus and selection in G418 resulted in stable downregulation of MUC1 expression. As a control, stable transfection of ZR-75-1 cells with the empty reterovirus had no effect on MUC1 expression. Cisplatin (CDDP) treated ZR-75-1/vector cells exhibited little if any decrease in mitochondrial transmembrane potential (ΔΨm). By contrast, CDDP treatment of ZR-75-1/MUC1siRNA cells was associated with a clear loss of ΔΨm. In concert with these findings, cytochrome C release, as measured by immunoblot, was attenuated in CDDP-treated ZR-75-1/vector cells, as compared to ZR-75-1/MUC1siRNA cells. In the absence of exposure to a cytotoxic agent, ZR-75-1 cells expressing the empty viral vector or MUC1siRNA exhibited less than 5% apoptosis. Treatment of ZR-75-1/vector cells with CDDP was associated with the induction of approximately 25% apoptosis. Notably, treatment of ZR-75-1/MUC1siRNA cells with CDDP resulted in over 60% apoptosis. The ZR-75-1 cells were also treated with different concentrations of etoposide. The results showed that sensitivity to 10 and 50 μM etoposide was increased substantially by knocking-down MUC1 expression.

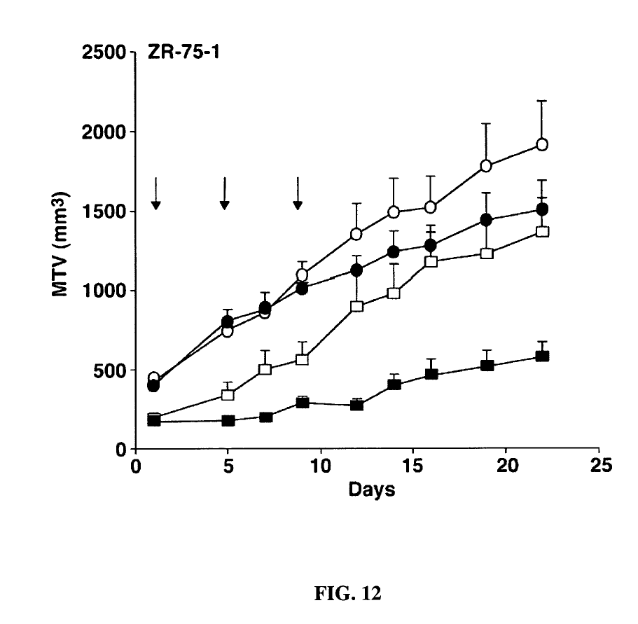

To determine if knocking-down MUC1 affects chemosensitivity in vivo, mice were injected with ZR-75-1 cells stably expressing the empty viral vector or MUCsiRNA. Growth of the ZR-75-1/MUC1siRNA was somewhat slowed compared to that found for ZR-75-1/vector cells. Treatment with CDDP was associated with a partial slowing of ZR-75-1/vector tumor growth (FIG. 12). By contrast, the ZR-75-1/MUC1siRNA tumors were considerably more sensitive to CDDP treatment (FIG. 12).

### Example 15

### MUC1 C-ter Localizes to Mitochondria

Cell culture. Human HCT116 colon carcinoma cells (ATCC, Manassas, Va.) were cultured in Dulbecco's modified Eagle's medium/F12 with 10% heat-inactivated fetal bovine serum, 100 units/ml penicillin, 100 mg/ml streptomycin and 2 mM L-glutamine. Cells were treated with EGF (10 ng/ml; Calbiochem-Novabiochem, San Diego, Calif.), HRG (20 ng/ml; Calbiochem-Novabiochem), cisplatin (CDDP; Sigma), etoposide (Sigma), rhTNF-a (Promega, Madison, Wis.), CHX (Sigma) or rhTRAIL (100 ng/ml; Calbiochem-Novabiochem).

Cell transfections. HCT116 cells were transfected with pIRES-puro2, pIRESpuro2-MUC1 or pIRES-puro2-MUC1(Y46F) as described (Li et al., 2001a). SW480 cells were transfected with pIRES-puro2 or pIRES-puro2-MUC1. Stable transfectants were selected in the presence of 0.4 mg/ml puromycin (Calbiochem-Novabiochem, San Diego, Calif.). Two independent transfections were performed for each vector.

Single cell clones were isolated by limiting dilution and expanded for analysis. In other studies, HCT116 cells were transiently transfected with the pEGFP-C1 vector (Clontech) in which MUC1 C-ter was cloned downstream to sequences encoding the green fluorescence protein (GFP).

Immunoblot analysis. Lysates were prepared from subconfluent cells as described (Li et al., 2001a). Equal amounts of protein were separated by SDS-PAGE and transferred to nitrocellulose membranes. The immunoblots were probed with anti-MUC1 N-ter (DF3) (Kufe et al., 1984), anti-MUC1 C-ter (Ab5; Neomarkers, Fremont, Calif.), anti-MUC1 C-ter (rabbit polyclonal DF3E) (Li et al., 2001), anti-MUC1 C-ter (human monoclonal ECD1), anti-b-actin (Sigma), anti-HSP60 (Stressgen Biotechnologies, Victoria, BC, Canada), anti-PCNA (Calbiochem-Novabiochem, San Diego, Calif.), anti-IkBa (Santa Cruz Biotechnology, Santa Cruz, Calif.), anti-calreticulin (Stressgen Biotechnologies; Victoria, BC, Canada), anti-PDGFR (Santa Cruz Biotechnology), anti-cytochrome c (BD PharMingen, San Diego, Calif.), anti-caspase-3 (BD PharMingen), anti-PKCd (Santa Cruz Biotechnology) anti-Smac/DIABLO (Medical & Biological Laboratories, Ltd., Japan) or anti-AIP (Santa Cruz Biotechnology). The immunocomplexes were detected with horseradish peroxidase-conjugated secondary antibodies and enhanced chemiluminescence (ECL, Amersham Biosciences, Piscataway, N.J.). Intensity of the signals was determined by densitometric scanning.

Flow cytometry. Cells were incubated with anti-MUC1 N-ter or control mouse IgG for 1 h at 4° C., washed, incubated with goat anti-mouse Ig fluorescein-conjugated antibody (Jackson Immunoresearch laboratories, West Grove, Pa.) and then fixed in 2% formaldehyde/PBS. Reactivity was detected by FACScan.

Confocal microscopy. Cells cultured on coverslips were incubated in Dulbecco's modified Eagle's/F12 medium containing 100 nM MitoTracker Red CMXRos (Molecular Probes, Eugene, Oreg.) for 20 min at 37° C. After staining, the cells were washed with fresh growth medium, pre-fixed in 3.7% formaldehyde/growth medium for 15 min at 37° C., washed with PBS, permeabilized in PBS containing 0.2% Triton X-100 for 5 min at 25° C., washed with PBS, then post-fixed in 3.7% formaldehyde/PBS for 5 min at 25° C. After several washes with PBS, the cells were blocked with 10% goat serum for 1 h at 25° C., stained with anti-MUC1 C-ter antibody for 1.5 h at 25° C., washed with PBS, incubated with FITC-conjugated secondary antibody (Jackson ImmunoResearch Laboratories, West Grove, Pa.) for 40 min at 25° C., washed with PBS and incubated with 2 mM TO-PRO3 (Molecular Probes) for 10 min at 25° C. After mounting the coverslips, images were captured with a LSM510 confocat microscope (ZEISS) at 1024×1024 pixel resolution. The excitation wavelength for FITC, MitoTracker Red and TO-PRO3 were 488 nm, 543 nm and 633 nm, respectively. Fluorescein fluorescence was captured through a 505- to 530-nm band-pass filter. MitoTracker Red CMXRos fluorescence was collected through a 560- to 615-nm band-pass filter. TO-PRO3 staining was visualized through a 650-nm long-pass filter.

Subcellular fractionation. Purified mitochondria and cytoplasmic lysates were prepared as described (Kumar et al., 2003). Cell membranes were purified from supernatants after sedimentation of nuclei and mitochondria as described (Kharbanda et al., 1996).

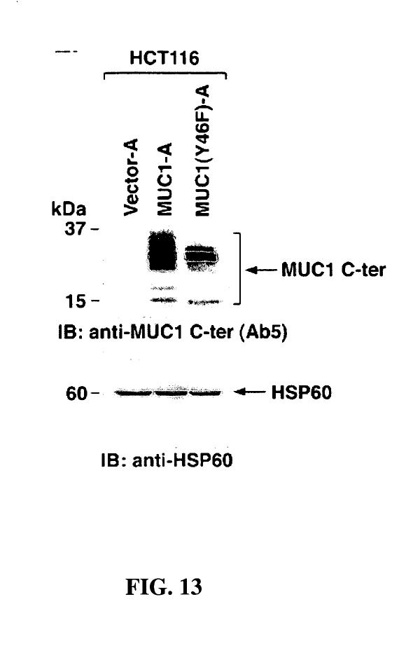

Results. MUC1-negative HCT116 cells were transfected to stably express the empty vector, MUC1 or MUC1 (Y46F) mutant. Two clones (A and B) of each were selected from independent transfections. Immunoblot analysis with anti-MUC1 demonstrated no detectable expression of the MUC1-N-ter or C-ter subunits in the vector transfectants. By contrast, MUC1N-ter expression was similar in cells transfected with MUC1 or MUC1(Y46F). Similar levels of MUC1 C-ter were also found in the MUC1 and MUC1(Y46F) tranfectants. To assess whether MUC1 is expressed at the cell membrane, the transfectants were analyzed by flow cytometry with the anti-MUC1 N-ter antibody. In contrast to HCT116/vector cells, MUC1 was detectable on the surface of HCT116 cells expressing MUC1 or MUC1(Y46F). To further define the distribution of MUC1, confocal microscopy was performed with antibodies against the MUC1 N-ter and C-ter. Both subunits were detectable at the cell membrane of the MUC1 transfectants. Unexpectedly, however, the MUC1 C-ter, and not the N-ter, were also expressed in a pattern that suggested mitochondrial localization. Indeed, colocalization of the MUC C-ter and MitoTracker supported targeting of MUC1 C-ter to mitochondria. By contrast, there was substantially less mitochondrial localization of the MUC1(Y46F) C-ter. Higher magnification and focusing of images within a single HCT116/MUC1 cell showed clear localization of MUC1 C-ter at the cell membrane and with Mitotracker throughout the mitochondrial network. Notably, detection of MUC1 C-ter at the cell membrane is not evident when focusing the confocal microscope of the mitochondria. To confirm these findings, mitochondrial lysates from the transfectants were subjected to immunoblot analyses with anti-MUC1 C-ter. The results demonstrate that the C-ter is detectable in the mitochondrial fraction from HCT116/MUC1, but not from HCT116/vector cells (FIG. 13). Moreover, in concert with the confocal data, mitochondrial localization in the MUC1(Y46F) C-ter was considerably less than that found for the MCU1 C-ter (FIG. 13). Equal loading of mitochondrial lysates were confirmed by immunoblotting for the mitochondrial HSP60 protein. The absence of the N-ter indicated that the mitochondrial fraction was not contaminated with cell membranes. Immunoblot analyses of the mitochondrial lysates with antibodies against the cytosolic IKBα, nuclear PCNA and endoplasmic reticulum-associated calreticulin proteins further indicated that the mitochondria are not significantly contaminated with these subcellular fractions.

To compare MUC-1 C-ter expression at the cell membrane with that in mitochondria, lysates from these fractions were subjected to immunoblot analysis with antibodies directed against the extracellular domain (ECD) and cytoplasmic domain (CD). The results obtained with Ab5 antibody which reacts with the C-terminal 17 amino acids of MUC1 CD, demonstrated similar patterns for MUC1 C-ter expressed at the cell membrane and in mitochondria. Reactivity with Ab5 was observed predominantly at 20-25 kDa. Reactive bands were also observed at approximately 17 and 15 kDa. Immunoblotting with DF3E antibody, which was generated against the VETQFNYKTEAAS motif as described in Example 2, demonstrated activity with lysates from both the cell membrane and mitochondria. Notably, reactivity of the DF3E antibody with only the 20-25 kDa MUC1 C-ter and the 17 kDa fragments indicated that the 15 kDa fragment, as detected with Ab5, does not contain the DF3E epitope. Another anti-MUC1 ECD antibody, designated ECD1, reacted predominantly with the 20-25 kDa MUC1 C-ter in both the cell membrane and mitochondria. These results suggest that the 17 kDa and 15 kDa fragments represent cleavage within the ECD. As controls, MUC1 N-ter expression was detectable only in the cell membrane fraction and HSP60 expression was restricted to the mitochondrial fraction. Moreover, there was no detectable contamination of the mitochondrial fraction with IkBa, PCNA or calreticulin.

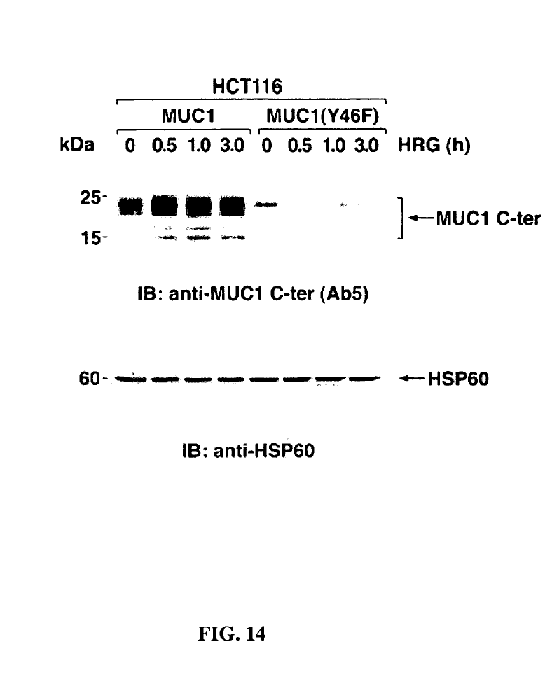

To extend these findings, MUC1 C-ter was expressed with a GFP tag at the N-terminus and assessed mitochondrial localization. Immunoblot analysis of mitochondrial lysates with anti-GFP and anti-MUC1 C-ter confirmed mitochondrial targeting of the GFP-tagged MUC1 C-ter fusion protein. As controls, expression of the platelet-derived growth factor receptor (PDGFR) and HSP60 was restricted to the cell membrane and mitochondrial fractions, respectively. The results of confocal studies also demonstrate colocalization of GFP-MUC1 C-ter with MitoTracker. The transfection efficiency of HCT116 cells is ˜25% under these experimental conditions (Ren et al., 2002). As a control, the prominent pattern of mitochondrial localization was not apparent when expressing GFP alone. These findings collectively demonstrate that MUC1 C-ter localizes to mitochondria. MUC1 C-ter is targeted to the nucleus with β-catenin in cells stimulated with EGF (Li et al., 2001a; Li et al., 2003). Stimulation of HCT116/MUC1 or HCT116/MUC1(Y46F) cells with EGF, however, had little effect on mitochondrial targeting of MUC1 C-ter. In contrast to EGF, HRG activates ErbB2 in the response of epithelial cells to stress (Vermeer et al., 2003) and targets MUC1 C-ter to the nucleolus (Li et al., 2003). Significantly, HRG treatment for 0.5 h was associated with a 2.3-fold increase in localization of MUC1 C-ter to mitochondria and this response persisted through 3 h (FIG. 14). Moreover, HRG had little effect on mitochondrial localization of MUC1(Y46F) C-ter (FIG. 14). Similar results were obtained in 3 separate experiments. In addition, there was no detectable β-catenin or γ-catenin in the mitochondrial fractions from control or HRG-stimulated cells. These findings indicate that targeting of MUC1 to mitochondria is regulated, at least in part, by HRG-induced signaling and that the Y46F mutation attenuates this response.

### Example 16

### MUC1 Attenuates Cytochrome C Release and Caspase-3 Activation

Methods. Experimental procedures and methods were as described in Example 15.

Results. Treatment of cells with DNA-damaging agents is associated with release of mitochondrial cytochrome c and activation of the intrinsic apoptotic pathway. To determine if mitochondrial localization of the MUC1 C-ter affects cytochrome c release, the HCT116 transfectants were treated with cisplatin (CDDP). Treatment of HCT1 16/vector cells with CDDP was associated with increased levels of cytosolic cytochrome c. Notably, expression of MUC1 significantly attenuated the release of cytochrome c. By contrast, expression of MUC1(Y46F) was ineffective in blocking CDDP-induced cytochrome c release. Similar results were obtained in the other separately isolated B clones. Release of cytochrome c in the response to genotoxic stress is associated with activation of caspase-3 and cleavage of PKCδ (Emoto et al., 1995). To assess the effects of MUC1 on caspase-3 activation, CDDP-treated cells were analyzed for cleavage of pro-caspase-3. The results demonstrate that, compared to HCT116/vector cells, MUC1 attenuates CDDP-induced activation of caspase-3. Cleavage of pro-caspase-3 in CDDP-treated HCT116/MUC1(Y46F) cells was similar to that in HCT116/vector cells. In concert with these results, caspase-3-mediated cleavage of PKCδ was attenuated in CDDP-treated HCT116/MUC1, as compared to HCT116/vector and HCT116/MUC1(Y46F), cells. Smac/DIABLO is a mitochondrial protein that induces caspase-dependent cell death by interacting with inhibitor of apoptosis proteins (IAPs) and blocking their caspase inhibitory activity (Du et al., 2000; Verhagen et al., 2000). To determine if MUC1 attenuates release of Smac/DIABLO, HCT116/vector, HCT116/MUC1 and HCT116/MUC(Y46F) cells were treated with CDDP for 24, 48 and 72 h, and cytosolic lysates were subjected to immunoblot analysis. The results demonstrate that, like cytochrome c, release of Smac/DIABLO is attenuated in HCT116/MUC1, as compared to HCT116/vector and HCT116/MUC1 (Y46F) cells. In addition, MUC1 attenuated release of the mitochondrial caspase-independent death effector, apoptosis-inducing factor (AIF) (Susin et at., 1999), as compared to that in cells expressing the vector or MUC1(Y46F). CDDP treatment of HCT116/vector and HCT116/MUC1(Y46F) cells for 72 h was associated with>90% cell death and decreases in the β-actin signals used as a control for loading. By contrast, treatment of HCT116/MUC1 cells with CDDP for 72 h was associated with cessation of cell growth and<30% cell death. These findings indicate that mitochondrial localization of MUC1 C-ter attenuates DNA damage-induced activation of the intrinsic apoptotic pathway.

### Example 17

### MUC1 Blocks DNA Damage- and TRAIL-Induced Apoptosis

Methods. Apoptotic cells were quantified by analysis of sub-G1 DNA and TUNEL staining. To assess sub-G1 DNA content, cells were harvested, washed with PBS, fixed with 80% ethanol, and incubated in PBS containing 20 ng/ml RNase (Roche) for 60 min at 37° C. Cells were then stained with 40 mg/ml propidium iodide (Sigma) for 30 min at room temperature in the dark. DNA content was analyzed by flow cytometry (EPICS XL-MCL, Coulter Corp.). Apoptotic cells with DNA fragmentation were detected by staining with the In Situ cell death detection kit (TUNEL; Roche Applied Science) and visualized by confocal microscopy (ZEISS LSM510). After staining, cells were analyzed by flow cytometry. Other experimental procedures and methods were as described in Example 15.

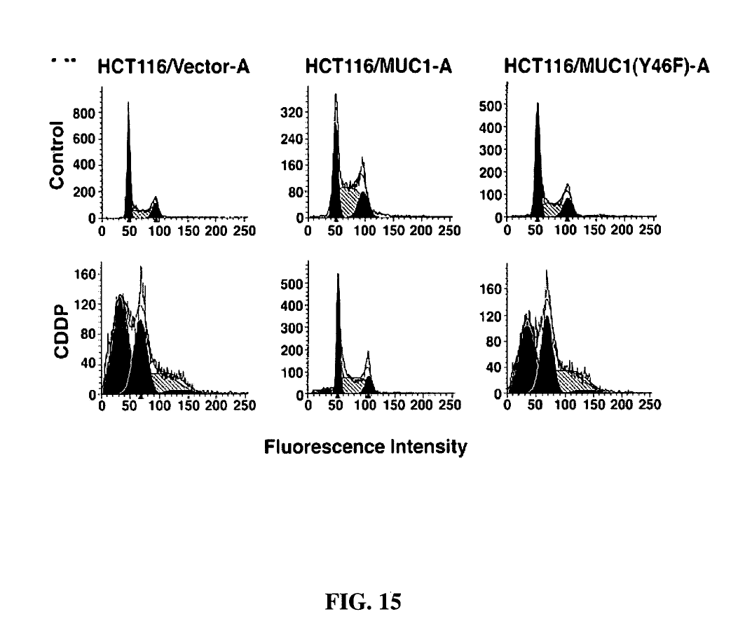

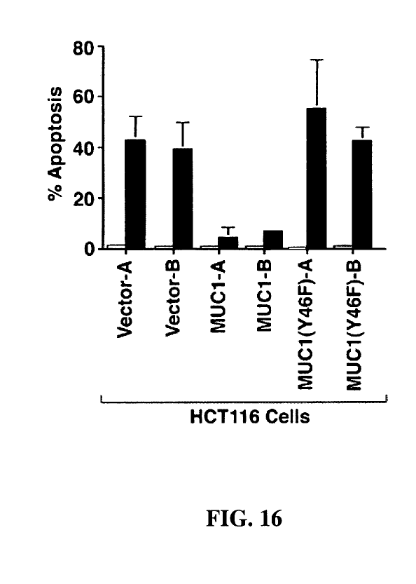

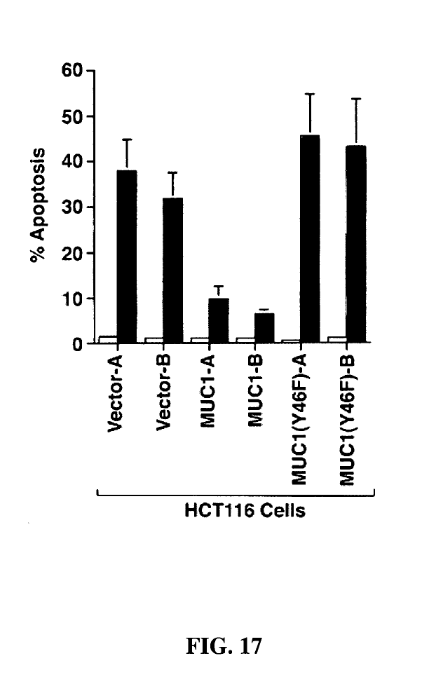

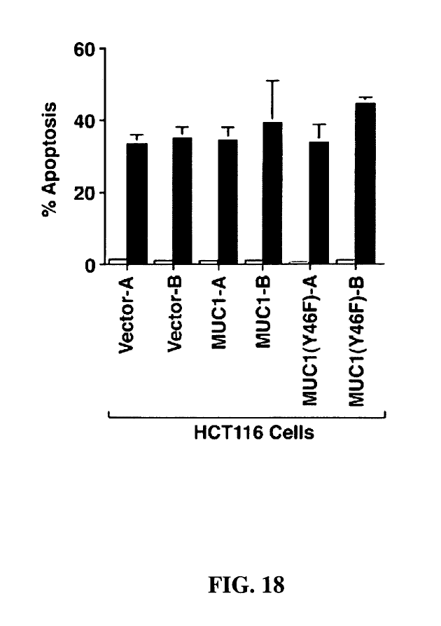

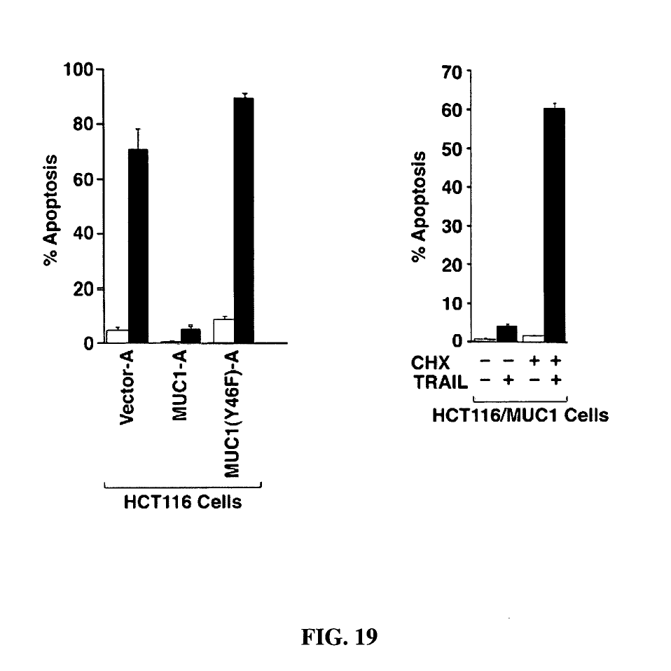

Results. To determine if MUC1 affects the induction of apoptosis by CDDP, cells were analyzed for sub-G1 DNA content. Treatment of HCT116/vector cells with CDDP for 24 h was associated with approximately 40% apoptosis (FIG. 15). Significantly, CDDP-induced apoptosis was attenuated in HCT116/MUC1, but not in HCT116/MUC1(Y46F), cells (FIG. 15). The attenuation of apoptosis by MUC1 as determined by cells with sub-G1 DNA content was confirmed when using TUNEL staining as an alternative method. In addition, similar results were obtained in multiple experiments with the separately isolated HCT116 cell clones (FIG. 16). Expression of wild-type MUC1, but not the MUC1(Y46F) mutant, also blocked apoptosis induced by the genotoxic agent, etoposide (FIG. 17). Stimulation of cell surface death receptors with TNF-α or the TNF-related apoptosis inducing factor TRAIL is associated with activation of the extrinsic apoptotic pathway. To determine if MUC1 affects death receptor-induced apoptosis, HCT116 cells were treated with TNF-α. In concert with previous work (Tsuchida et at., 1995), TNF-α alone failed to induce apoptosis of HCT116/vector cells. However, treatment with TNF-α in the presence of cycloheximide (CHX) was associated with induction of HCT116/vector cell apoptosis (FIG. 18). Similar results were obtained when HCT116/MUC1 and HCT116/MUC1(Y46F) cells were treated with TNF-α and CHX (FIG. 18), indicating that MUC1 has no effect on TNF-α+CHX-induced apoptosis. By contrast, TRAIL was effective in inducing apoptosis of HCT116/vector cells without adding CHX and, importantly, MUC1, but not MUC1(Y46F), attenuated this response (FIG. 19). Moreover, when HCT116/MUC1 cells were treated with TRAIL+CHX, MUC1 was ineffective in attenuating TRAIL-induced apoptosis (FIG. 19). Of note, CHX had no apparent effect on expression of MUC1 C-ter. These findings indicate that i) mitochondrial localization of MUC1 attenuates apoptosis induced by activation of the intrinsic pathway and ii) MUC1 attenuates TRAIL-induced apoptosis by a mechanism that may be mediated by a short-lived protein.

### Example 18

### Diverse Carcinomas Express the MUC1 C-ter in Mitochondria

Methods. Human HCT116 and SW480 colon carcinoma cells (ATCC, Manassas, Va.) were cultured in Dulbecco's modified Eagle's medium/F12 with 10% heat-inactivated fetal bovine serum, 100 units/ml penicillin, 100 mg/ml streptomycin and 2 mM L-glutamine. Human A549 lung, T-47D breast and ZR-75-1 breast carcinoma cells (ATCC) were grown in RPMI 1640 medium containing 10% heat-inactivated fetal bovine serum, antibiotics and L-glutamine. Insulin (10 ng/ml; Life Technologies, Rockville, Md.) was also added to cultures of the T-47D cells. Other experimental procedures and methods were as described in Example 15.

Results. To determine if other carcinomas exhibit mitochondrial localization of the MUC1 C-ter, we first performed confocal immunofluorescence studies on human SW480 colon carcinoma cells stably transfected to express an empty vector or MUC1. SW480 cells transfected with the empty vector expressed a low level of MUC1 and exhibited little if any MUC1 C-ter in mitochondria. By comparison, SW480/MUC1 cells exhibited substantially higher levels of MUC1 expression and clear colocalization of MUC1 C-ter with MitoTracker. To extend these observations to carcinomas that endogenously express MUC1, we performed similar studies on human lung and breast cancer cells. There was no detectable MUC1 N-ter in mitochondria of A549 lung carcinoma cells. However, as observed in MUC1 transfectants, the MUC1 C-ter was detectable in mitochondria. Similar results were obtained with T-47D and ZR-75-1 breast carcinoma cells. These findings indicate that the MUC1 C-ter localizes to mitochondria in both MUC1 transfectants and carcinomas that endogenously express MUC1.

### Example 19

### MUC1 Activates the Anti-Apoptotic PI3K/Akt and Bcl-X_{L} Pathways Methods

Cell culture. Rat 3Y1 fibroblasts stably expressing the empty vector or full-length MUC1 (19) were cultured in Dulbecco's modified Eagle's medium containing 10% heat-inactivated fetal calf serum, 100 units/ml penicillin, 100 mg/ml streptomycin and 2 mM L-glutamine. Cells were treated with 25 mM LY294002 (Sigma), 10 mM 1-b-D-arabinofuranosycyltosine (ara-C; Sigma) or 50 mM gemcitabine (Eli Lilly).

Immunoblot analysis. Whole cell lysates and cytoplasmic fractions were prepared from subconfluent cells as described (Li et all., 1998; Li et al., 2001). Proteins were separated by SDS-PAGE, transferred to nitrocellulose membranes and probed with anti-phospho-Akt (Cell Signaling, Beverly, Mass.), anti-Akt (Cell Signaling), anti-b-actin (AC-15; Sigma), anti-phospho-Bad (Cell Signaling), anti-Bad (Santa Cruz Biotechnology, Santa Cruz, Calif.), anti-PI3K p85 (Upstate Biotechnology, Lake Placid, N.Y.), anti-PTEN (Ramaswamy et al., 1999), anti-Bcl-x_{L} (Santa Cruz Biotechnology), anti-Bcl-2 (BD PharMingen, San Diego, Calif.), anti-HSP60 (Stressgen, Victoria BC, Canada), anti-cytochrome c (Kirken et al., 1995), anti-caspase-9 (H-83; Santa Cruz) and anti-PKCd (C-20; Santa Cruz).

Assessment of mitochondrial transmembrane potential. Cells were incubated with 50 ng/ml Rhodamine 123 (Molecular Probes, Eugene, Oreg.) for 15 min at 37° C. After washing with PBS, samples were analyzed by flow cytometry using 488 nm excitation and measurement of emission through a 575/26 (ethidium) bandpass filter.

Isolation of mitochondria. Mitochondria were purified as described (Kumar et al., 2003).

Apoptosis assays. For TUNEL staining, cells were fixed in 4% paraformaldehyde, permeabilized in 0.1% Triton X-100/0.1% sodium citrate for 2 min and processed according to the manufacturer's instructions (In Situ Cell Death detection kit, fluorescein; Roche). For analysis of sub-G1 DNA, cells were washed with PBS, fixed in 70% ethanol, incubated with 2.5 mg/ml propidium iodide and 50 mg/ml RNase, and then monitored by FACScan.

Results

MUC1 activates the PI3K/Akt signaling pathway. To determine if MUC1 affects the phosphoinositide3-kinase(PI3K)/Akt pathway, lysates from 3Y1 cells stably expressing the empty vector or MUC1 were subjected to immunoblotting with anti-phospho-Akt. Levels of phospho-Akt were substantially increased in 3Y1/MUC1 cells compared to that in 3Y1/vector cells. Similar results were obtained in separately isolated 3Y1/vector and 3Y1/MUC1 clones. The demonstration that MUC1 has little if any effect on Akt protein levels indicated that Akt is activated in the 3Y1/MUC1 cells. Akt phosphorylates and inactivates the pro-apoptotic function of Bad (Datta et at., 1997). Immunoblot analysis of the lysates with an anti-phospho-Bad antibody demonstrated that MUC1 markedly increases levels of phosphorylated Bad. As a control, MUC1 had little effect on Bad protein levels. Akt is regulated in part by P13K-mediated formation of PI(3,4,5)P3 and degradation of this PI by the PTEN phosphatase. To determine if MUC1 affects expression of P13K, lysates were probed with an anti-PI3K p85 antibody. MUC1 had no detectable effect on P13K p85 expression. Moreover, PTEN levels were similar in the 3Y1/vector and 3Y1 /MUC1 cells. Significantly, however, treatment of the 3Y1/MUC1 cells with the P13K inhibitor, LY294002, was associated with down-regulation of both phospho-Akt and phospho-Bad levels. These findings indicate that MUC1 activates the PI3K/Akt pathway.

MUC1 upregulates Bcl-x_{L} expression. Bad promotes apoptosis by forming a heterodimer with Bcl-x_{L} (Datta et at., 1997). To determine if MUC1 also affects Bcl-x_{L} expression, lysates were analyzed by immunoblotting with anti-Bcl-x_{L}. Levels of Bcl-x_{L} were increased in 3Y1/MUC1 cells as compared to 3Y1/vector cells. The demonstration that MUC1 has little effect on Bcl-2 levels indicated that MUC1 selectively upregulates Bcl-x_{L} expression. The findings that Bcl-x_{L} levels are down-regulated by LY294002 in Baf-3 hematopoietic cells (Leverrier et al., 1999), and not in PC-3 prostate cancer cells (Yang & Lattime et al., 2003), have indicated that Bcl-x_{L} expression is controlled by P13K-dependent and -independent mechanisms. Treatment of 3Y1/MUC1-A cells with LY294002 had little if any effect on Bcl-x_{L} levels. Similar results were obtained with LY294002-treated 3Y1/MUC1-B cells. Other studies have shown that LY294002 down-regulates MUC1 in gastric carcinoma cells (Kobayashi et al., 2003). By contrast, the LY294002 had no apparent effect on MUC1 expression in 3Y1/MUC1 cells, indicating that MUC1 is regulated by PI3K-independent pathways. Bcl-x_{L} localizes to the outer mitochondrial membrane where it interacts with Bad (Boise et al., 1993; Vander Heiden et al., 1997). In this context, Bcl-x_{L} levels were increased in mitochondria of 3Y1/MUC1, as compared to 3Y1/vector, cells. To determine if MUC1 also localizes to the outer mitochondrial membrane, lysates from mitochondria were analyzed by immunoblotting with antibodies against the MUC1 N-ter and C-ter subunits. There was no detectable MUC1 N-ter or C-ter in the mitochondrial lysates. Immunoblot analysis of the mitochondrial HSP60 protein was also included as a control for equal loading of the lanes. These results indicate that MUC1 increases mitochondrial Bcl-x_{L} levels by a PI3K-independent mechanism.

MUC1 blocks activation of the intrinsic apoptotic pathway. Increases in phospho-Bad and Bcl-x_{L} protect against apoptosis induced by activation of the intrinsic mitochondrial pathway (Van Heiden et al., 1997; Datta et al., 1995). To assess the effects of MUC1 on chemosensitivity, we treated cells with ara-C, an antimetabolite that induces release of mitochondrial cytochrome c and thereby apoptosis (Major et al., 1981; Kojima et al., 1998). Exposure of 3Y1/vector-A and -B cells to ara-C was associated with a decrease in mitochondrial transmembrane potential (FIG. 3A). By contrast and as found previously in Bcl-xL expressing cells (Vander Heiden et at, 1997), ara-C treatment of 3Y1/MUC1 cells was associated with an apparent increase in mitochondrial transmembrane potential. Ara-C treatment was also associated with release of cytochrome c into the cytosol of 3Y1/vector, and not 3Y1/MUC1, cells. In concert with these results, activation of caspase-9 was attenuated in 3Y1/MUC1, as compared to 3Y1/vector, cells. Moreover, caspase-3-mediated cleavage of PKCd (Emoto et at., 1995) was attenuated in the MUC1 expressing cells. Similar results were obtained in the separately isolated 3Y1/vector-B and 3Y1/MUC1-B cells. These findings indicate that MUC1 attenuates ara-C-induced activation of the intrinsic apoptotic pathway.

MUC1 attenuates ara-C- and gemcitabine-induced apoptosis. To determine if MUC1 affects induction of apoptosis by ara-C, the 3Y1/vector and 3Y1/MUC1 cells were analyzed for TUNEL staining and sub-G1 DNA. Treatment with ara-C was associated with a substantial induction of TUNEL staining in 3Y1/vector, but not 3Y1/MUC1, cells. Moreover, ara-C treatment of 3Y1/vector cells was associated with over 40% apoptosis as determined by sub-G1 DNA analysis. By contrast, ara-C-induced apoptosis was significantly attenuated in the 3Y1/MUC1 cells. Similar results were obtained in multiple experiments with the 3Y1/MUC1-A cells and in the other separately isolated 3Y1/MUC1-B clone.

To extend these observations to other anti-cancer agents, we treated the 3Y1/vector and 3Y1/MUC1 cells with gemcitabine. As determined by analysis of sub-G1 DNA and as found with ara-C, gemcitabine-induced apoptosis was attenuated in 3Y1/MUC1, as compared to 3Y1/vector, cells. Similar findings were obtained when the cells were analyzed by TUNEL staining (data not shown). These results were confirmed in multiple experiments with the 3Y1/MUC1-A clone. Moreover, similar results were obtained with the 3Y1/MUC1-B cells. These findings demonstrate that MUC1 attenuates the induction of apoptosis by ara-C and gemcitabine.

The present invention has been shown by both description and examples. The Examples are only examples and cannot be construed to limit the scope of the invention. One of ordinary skill in the art will envision equivalents to the inventive process described by the following claims that are within the scope and spirit of the claimed invention.

### References

The following references, to the extent that they provide exemplary procedural or other details supplementary to those set forth herein, are specifically incorporated herein by reference.
Barry & Sharkey, Hum. Pathol., 16:225-7, 1985.
Baruch et al., Int. J. Cancer, 71:741-749, 1997.
Bertrand et al., Biochem. Biophys. Res. Commun., 296:1000-10004, 2002.
Boise et al., Cell., 74 :597-608, 1993.
Brummelkamp et al., Science, 296:550-553, 2002.
Brunner et al., Nature, 385:829-833, 1997.
Crooke et al., J. Pharmacol. Exp. Ther., 277:923-937, 1996.
Daniels & Reynolds, Mol. Cell. Biol., 19:3614-23, 1999.
Datta et al., Cell Growth Differ., 6:363-370, 1995.
Datta et al., Cell, 91:231-241, 1997.
Dawson et al., Science 266:776, 1994.
Devroe & Silver, BMC Biotechnol, 2:15, 2002.
Duffy, in Vaccine Preparation Techniques, Noyes Data Corporation of Park Ridge, N.J., 1980.
Elbashir et al., Methods, 26:199-213, 2002.
Emoto et al., EMBO, 14 :6148-6156, 1995.
Finn et al., Immunol. Rev. 145:61-89, 1995.
Fisher et al., J. Biol. Chem., 277:22980-22984, 2002.
Gansow, Int. J. Rad Appl. Instrum. [B], Nucl. Med. Biol. 18:369-381, 1991.
Goding, In: Monoclonal Antibodies: Principles and Practice, 2d ed., Orlando, Fla., Academic Press, pp. 60-61, 1986.
Harborth et al., J. Cell Sci., 114:4557-4565, 2001.
Hohjoh, FEBS Lett., 521:195-199, 2002.
Itzkowitz et al., Cancer, 66:1960-6, 1990.
Jawhari et al., J. Pathol., 189:180-85, 1999.
Jensen et al., Bioconjug. Chem., 13:975-984, 2002.
Jones et al., Nature 321:522-525, 1986.
Kabanov et al., FEBS Lett., 259:327-330, 1990.
Kirken et al., Prot. Exp. Purif., 6 :707-715, 1995.
Kharbanda et al., Cancer Res., 56:3617-3621, 1996.
Kobayashi et al., Oncogene, 22:1294-1301, 2003.
Kojima et al., 273:16647-16650, 1998.
Kufe et al., Hybridoma, 3:223-232, 1984.
Kumar et al., Mol. Pharmacol., 63:276-282, 2003.
LeBel et al., Chem. Res. Toxicol. 5:227-231, 1992.
Letsinger et al., Proc. Natl. Acad. Sci. USA, 86:6553-6556, 1989.
Leverrier et al., Celt Death. Differ., 6,290-296, 1999.
Li et al., Mol Cell Biol., 18:7216-24, 1998.
Li et al., J. Biol. Chem., 276:6061-64, 2001.
Li et al., J. Biol Chem, 276:35239-35242, 2001 a.
Li et al., Mol. Cancer Res., 1:765-775, 2003.
Li & Kufe, Biochem Biophys. Res. Commun., 281, 440-43, 2001.
Ligtenburger et al., Cancer Res., 15:2318-2324, 1992.
Manoharan et al., Ann. N.Y. Acad. Sci., 660:306-309, 1992
Manoharan et al., Bioorg. Med. Chem. Let., 3:2765-2770, 1993
Manoharan et al., Bioorg. Med. Chem. Let., 4:1053-1060, 1994.
Manoharan et al., Tetrahedron Lett., 36:3651-3654; 1995.
Manoharan et al., Nucleosides & Nucleotides,14:969-973, 1995.
Martinez et al., Cell, 110:563-574, 2002.
Mishra et al., Biochim. Biophys. Acta, 1264:229-237, 1995.
Molenaar et al., Cell, 86:391-399, 1996.
Novak & Dedhar, Cell Mol. Life Sci., 523-37, 1999.
Oberhauser et al., Nucl. Acids Res., 20:533-538, 1992.
Oehlke et al., Eur. J. Biochem., 269:4025-4032, 2002.
Olsnes & Pihl, Pharmac. Ther. 25:355-381, 1981.
Pandey et al., Cancer Res., 55:4000-4003, 1995.
Pastan et al., Ann. Rev. Biochem. 61:331-354, 1992.
Paul et al., Nature Biotechnol., 20:505-508, 2002.
Perey et al., Cancer Res., 52:2563-68, 1992.
Pluckthun in The Pharmacology of Monoclonal Antibodies, vol. 113:269-315, Rosenburg and Moore eds. Springer-Verlag, N.Y., 1994.
Presta, Curr. Op. Struct Biol. 2:593-596, 1992.
Ramaswamy et al, Proc. Nat'l Acad. Sci USA, 96:2110-2115, 1999.
Reddish et al., Cancer Immunol. Immunother., 42:303-9, 1996.
Ren et al., J. Biol. Chem., 277:17616-17622, 2002.
Reynolds et al., Mol. Cell. Biol. 9:629-38, 1989.
Reynolds et al., Mol. Cell. Biol., 14:8333-41, 1994.
Riechmann et al., Nature 332:323-329, 1988.
Ring et al., Human Genet., 104:326-32, 1999.
Saison-Behmoaras et al., EMBO J., 10:1111 -1118, 1991.
Sambrook et al., Molecular Cloning: A Laboratory Manual, Cold Spring Harbor Laboratory Press, Cold Spring Harbor, N.Y. 1989.
Shapiro, Methods, 21:271-279, 2000.
Shea et al., Nucl. Acids Res., 18:3777-3783, 1990.
Shimazui et al., Cancer Res., 56:4154-58, 1996.
Smith & Waterman, Adv. Appl. Math., 2:482-489, 1981.
Strouss & Decker, Crit. Rev. Biochem. Mol. Biol., 27:57-92, 1992.
Sui et al., Pro. Nat'l. Acad. Sci. U.S.A., 99,:5515-5520, 2002.
Susin et al., Nature, 397:441-446, 1999.
Svinarchuk et al., Biochimie, 75:49-54, 1993.
Takeichi, Annu. Rev. Biochem., 59:237-52, 1990.
Thorpe et al., Immunological Rev. 62:119-158, 1982.
Thorpe et al., Monoclonal Antibodies in Clinical Medicine, pp. 168-190 Academic Press, N.Y. 1982(a).
Tijsterman et al., 295:694-697, 2002.
Tsuchida et al., J. Immunot., 154 :2403-2412, 1995.
Waldmann, Science, 252:1657, 1991.
Vermeer et al., Nature, 422 :322-326, 2003.
Xia et al., Nature BiotechnoL., 20:1006-1010, 2002.
Yamamoto et al., J. Biol. Chem. 272:12492-94, 1997.
Yang et al., Mot. Cell. Biot., 21:7807-7816, 2001.
Yang & Lattime et al., Cancer Res., 63:2150-2157, 2003.
Yin et al., Mot. Cancer Therap., 1:43-48, 2001.
Yu et al., Proc. Nat'l. Acad. Sci. U.S.A, 99:6047-6052, 2002.
Yuan et al., PNAS, 94:1437-1440, 1997.
Zamore, Nature Struct. BioL., 8:746-750, 2001.
Zatsepin et al., Bioconjug. Chem. 13:822-830, 2002.
Zrihan-Licht, et al., FEBS Let., 356:130-36, 1994.
U.S. Pat. No. 4,675,382
U.S. Pat. No. 4,894,443
U.S. Pat. No. 5,506,343

1. A method of enhancing the induction apoptosis in a MUC1 expressing cancer cell by a pro-apoptotic chemotherapeutic agent, comprising first contacting said cancer cell with an effective amount of a MUC1/ECD antagonist and subsequently contacting said cancer cell with an effective amount of a pro-apoptotic chemotherapeutic agent.

2. The method of claim 1, wherein said pro-apoptotic chemotherapeutic agent is a DNA-interactive chemotherapeutic agent.

3. The method of claim 1, wherein said pro-apoptotic chemotherapeutic agent is a tubulin interactive chemotherapeutic agent.

4. The method of claim 1, wherein said pro-apoptotic chemotherapeutic agent is an antimetabolite chemotherapeutic agent.

5. The method of claim 1, wherein said MUC1/ECD antagonist is an agent that downregulates MUC1.

6. The method of claim 5, wherein said agent that downregulates MUC1 is an interference RNA molecule.

7. A method of decreasing the proliferation of a cancer cell comprising:
(a) identifying said cancer cell as a MUC1 expressing cancer cell;
(b) contacting said MUC1 expressing cancer cell with an effective amount of a MUC1/ECD antagonist; and
(c) subsequently contacting said MUC1 expressing cancer cell with an effective amount of a pro-apoptotic chemotherapeutic agent.

8. The method of claim 7, wherein said pro-apoptotic chemotherapeutic agent is a DNA-interactive chemotherapeutic agent.

9. The method of claim 7, wherein said pro-apoptotic chemotherapeutic agent is a tubulin interactive chemotherapeutic agent.

10. The method of claim 7, wherein said pro-apoptotic chemotherapeutic agent is an antimetabolite chemotherapeutic agent.

11. The method of claim 7, wherein said MUC1/ECD antagonist is an agent that downregulates MUC1.

12. The method of claim 8, wherein said agent that downregulates MUC1 is an interference RNA molecule.

13. A method of killing a MUC1 expressing cancer cell, wherein said MUC1 expressing cancer has been subjected to a first pro-apoptotic chemotherapeutic agent, comprising:
(a) contacting said MUC1 expressing cancer cell with an effective amount of an agent that downregulates MUC1; and
(b) subsequently contacting said MUC1 expressing cancer cell with an effective amount of a second pro-apoptotic chemotherapeutic agent.

14. The method of claim 13, wherein said first pro-apoptotic chemotherapeutic agent is a DNA-interactive chemotherapeutic agent, a tubulin interactive chemotherapeutic agent, or an antimetabolite chemotherapeutic agent.

15. The method of claim 13, wherein said second pro-apoptotic chemotherapeutic agent is a DNA-interactive chemotherapeutic agent, a tubulin interactive chemotherapeutic agent, or an antimetabolite chemotherapeutic agent.

16. The method of claim 13, wherein said first pro-apoptotic chemotherapeutic agent and said second pro-apoptotic chemotherapeutic agent are the same.

17. The method of claim 13, wherein said MUC1/ECD antagonist is an agent that downregulates MUC1.

18. The method of claim 17, wherein said agent that downregulates MUC1 is an interference RNA molecule.
